SEQUENCE LISTING (42 sequences)

SEQ ID NO: 1 - DNA, 738 bp, Homo sapiens
  Immature TFAM coding sequence, isoform 1
atggcgtttctccgaagcatgtggggcgtgctgagtgccctgggaaggtctggagcagagctgtgcaccggctgtggaagtcgactgcgctcccccttcagttttgtgtatttaccgaggtggttttcatctgtcttggcaagttgtccaaagaaacctgtaagttcttaccttcgattttctaaagaacaactacccatatttaaagctcagaacccagatgcaaaaactacagaactaattagaagaattgcccagcgttggagggaacttcctgattcaaagaaaaaaatatatcaagatgcttatagggcggagtggcaggtatataaagaagagataagcagatttaaagaacagctaactccaagtcagattatgtctttggaaaaagaaatcatggacaaacatttaaaaaggaaagctatgacaaaaaaaaaagagttaacactgcttggaaaaccaaaaagacctcgttcagcttataacgtttatgtagctgaaagattccaagaagctaagggtgattcaccgcaggaaaagctgaagactgtaaaggaaaactggaaaaatctgtctgactctgaaaaggaattatatattcagcatgctaaagaggacgaaactcgttatcataatgaaatgaagtcttgggaagaacaaatgattgaagttggacgaaaggatcttctacgtcgcacaataaagaaacaacgaaaatatggtgctgaggagtgt

SEQ ID NO: 2 - protein, 246 aa, Homo sapiens
  Immature TFAM protein, isoform 1
MAFLRSMWGVLSALGRSGAELCTGCGSRLRSPFSFVYLPRWFSSVLASCPKKPVSSYLRFSKEQLPIFKAQNPDAKTTELIRRIAQRWRELPDSKKKIYQDAYRAEWQVYKEEISRFKEQLTPSQIMSLEKEIMDKHLKRKAMTKKKELTLLGKPKRPRSAYNVYVAERFQEAKGDSPQEKLKTVKENWKNLSDSEKELYIQHAKEDETRYHNEMKSWEEQMIEVGRKDLLRRTIKKQRKYGAEEC

SEQ ID NO: 3 - DNA, 612 bp, Homo sapiens
  Mature TFAM coding sequence, isoform 1
tcatctgtcttggcaagttgtccaaagaaacctgtaagttcttaccttcgattttctaaagaacaactacccatatttaaagctcagaacccagatgcaaaaactacagaactaattagaagaattgcccagcgttggagggaacttcctgattcaaagaaaaaaatatatcaagatgcttatagggcggagtggcaggtatataaagaagagataagcagatttaaagaacagctaactccaagtcagattatgtctttggaaaaagaaatcatggacaaacatttaaaaaggaaagctatgacaaaaaaaaaagagttaacactgcttggaaaaccaaaaagacctcgttcagcttataacgtttatgtagctgaaagattccaagaagctaagggtgattcaccgcaggaaaagctgaagactgtaaaggaaaactggaaaaatctgtctgactctgaaaaggaattatatattcagcatgctaaagaggacgaaactcgttatcataatgaaatgaagtcttgggaagaacaaatgattgaagttggacgaaaggatcttctacgtcgcacaataaagaaacaacgaaaatatggtgctgaggagtgt

SEQ ID NO: 4 - protein, 204 aa, Homo sapiens
  Mature TFAM protein, isoform 1
SSVLASCPKKPVSSYLRFSKEQLPIFKAQNPDAKTTELIRRIAQRWRELPDSKKKIYQDAYRAEWQVYKEEISRFKEQLTPSQIMSLEKEIMDKHLKRKAMTKKKELTLLGKPKRPRSAYNVYVAERFQEAKGDSPQEKLKTVKENWKNLSDSEKELYIQHAKEDETRYHNEMKSWEEQMIEVGRKDLLRRTIKKQRKYGAEEC

SEQ ID NO: 5 - DNA, 642 bp, Homo sapiens
  Immature TFAM coding sequence, isoform 2
atggcgtttctccgaagcatgtggggcgtgctgagtgccctgggaaggtctggagcagagctgtgcaccggctgtggaagtcgactgcgctcccccttcagttttgtgtatttaccgaggtggttttcatctgtcttggcaagttgtccaaagaaacctgtaagttcttaccttcgattttctaaagaacaactacccatatttaaagctcagaacccagatgcaaaaactacagaactaattagaagaattgcccagcgttggagggaacttcctgattcaaagaaaaaaatatatcaagatgcttatagggcggagtggcaggtatataaagaagagataagcagatttaaagaacagctaactccaagtcagattatgtctttggaaaaagaaatcatggacaaacatttaaaaaggaaagctatgacaaaaaaaaaagaaaagctgaagactgtaaaggaaaactggaaaaatctgtctgactctgaaaaggaattatatattcagcatgctaaagaggacgaaactcgttatcataatgaaatgaagtcttgggaagaacaaatgattgaagttggacgaaaggatcttctacgtcgcacaataaagaaacaacgaaaatatggtgctgaggagtgt

SEQ ID NO: 6 - protein, 214 aa, Homo sapiens
  Immature TFAM protein, isoform 2
MAFLRSMWGVLSALGRSGAELCTGCGSRLRSPFSFVYLPRWFSSVLASCPKKPVSSYLRFSKEQLPIFKAQNPDAKTTELIRRIAQRWRELPDSKKKIYQDAYRAEWQVYKEEISRFKEQLTPSQIMSLEKEIMDKHLKRKAMTKKKEKLKTVKENWKNLSDSEKELYIQHAKEDETRYHNEMKSWEEQMIEVGRKDLLRRTIKKQRKYGAEEC

SEQ ID NO: 7 - DNA, 516 bp, Homo sapiens
  Mature TFAM coding sequence, isoform 2
tcatctgtcttggcaagttgtccaaagaaacctgtaagttcttaccttcgattttctaaagaacaactacccatatttaaagctcagaacccagatgcaaaaactacagaactaattagaagaattgcccagcgttggagggaacttcctgattcaaagaaaaaaatatatcaagatgcttatagggcggagtggcaggtatataaagaagagataagcagatttaaagaacagctaactccaagtcagattatgtctttggaaaaagaaatcatggacaaacatttaaaaaggaaagctatgacaaaaaaaaaagaaaagctgaagactgtaaaggaaaactggaaaaatctgtctgactctgaaaaggaattatatattcagcatgctaaagaggacgaaactcgttatcataatgaaatgaagtcttgggaagaacaaatgattgaagttggacgaaaggatcttctacgtcgcacaataaagaaacaacgaaaatatggtgctgaggagtgt

SEQ ID NO: 8 - protein, 172 aa, Homo sapiens
  Mature TFAM protein, isoform 2
SSVLASCPKKPVSSYLRFSKEQLPIFKAQNPDAKTTELIRRIAQRWRELPDSKKKIYQDAYRAEWQVYKEEISRFKEQLTPSQIMSLEKEIMDKHLKRKAMTKKKEKLKTVKENWKNLSDSEKELYIQHAKEDETRYHNEMKSWEEQMIEVGRKDLLRRTIKKQRKYGAEEC

SEQ ID NO: 9 - DNA, 1188 bp, Homo sapiens
  Immature TFB2M coding sequence, isoform 1
atgtggatcccagtggtcgggcttcctcggcggctgaggctctccgccttggcgggcgctggtcgcttttgcattttagggtctgaagcggcgacgcgaaagcatttgccggcgaggaaccactgtgggctctctgactcctctccgcagctgtggcccgaaccggatttcaggaatccgccaaggaaggcgtctaaggccagcttagactttaagcgttacgtaaccgatcggagattggctgagaccctggcgcaaatctatttgggaaaaccaagtagacctccacacctactgctggagtgcaatccaggtcctggaatcctgactcaggcattacttgaagctggtgccaaagtggttgcgctcgaaagtgacaaaacttttattccacatttggagtccttaggaaaaaatctggatggaaaactacgagtgattcactgtgacttctttaaactagatcctagaagtggtggagtaataaaaccacctgctatgtcttctcgagggctctttaagaatttgggaatagaagcagttccttggacagcagacatccctttaaaagtagttggaatgttcccaagtagaggtgagaaaagggcactttggaaactcgcatatgacttgtattcctgtacttctatatataaatttggacgaatagaagtaaatatgtttattggtgaaaaagaattccagaaactaatggcagatcccggaaatccagacttgtatcatgtattaagtgttatctggcaattagcttgtgagattaaggttctgcacatggagccttggtcatcatttgatatatacacccggaaagggccgctggaaaacccaaagcgtagggaattattagaccaattacaacaaaagctgtatcttattcaaatgattcctcgtcaaaatttatttaccaagaacttaacacctatgaactataatatattttttcacttgttaaagcactgttttgggaggcgcagcgccactgtaatagaccacttacgttcattgactccacttgatgcgagagatatattgatgcaaataggaaaacaggaggatgagaaagtagttaacatgcaccctcaagacttcaaaacactttttgaaactatagagcgttccaaagattgtgcttataaatggctgtatgatgaaaccctggaagatagg

SEQ ID NO: 10 - protein, 396 aa, Homo sapiens
  Immature TFB2M protein, isoform 1
MWIPVVGLPRRLRLSALAGAGRFCILGSEAATRKHLPARNHCGLSDSSPQLWPEPDFRNPPRKASKASLDFKRYVTDRRLAETLAQIYLGKPSRPPHLLLECNPGPGILTQALLEAGAKVVALESDKTFIPHLESLGKNLDGKLRVIHCDFFKLDPRSGGVIKPPAMSSRGLFKNLGIEAVPWTADIPLKVVGMFPSRGEKRALWKLAYDLYSCTSIYKFGRIEVNMFIGEKEFQKLMADPGNPDLYHVLSVIWQLACEIKVLHMEPWSSFDIYTRKGPLENPKRRELLDQLQQKLYLIQMIPRQNLFTKNLTPMNYNIFFHLLKHCFGRRSATVIDHLRSLTPLDARDILMQIGKQEDEKVVNMHPQDFKTLFETIERSKDCAYKWLYDETLEDR

SEQ ID NO: 11 - DNA, 1059 bp, Homo sapiens
  Mature TFB2M coding sequence, isoform 1
ctctctgactcctctccgcagctgtggcccgaaccggatttcaggaatccgccaaggaaggcgtctaaggccagcttagactttaagcgttacgtaaccgatcggagattggctgagaccctggcgcaaatctatttgggaaaaccaagtagacctccacacctactgctggagtgcaatccaggtcctggaatcctgactcaggcattacttgaagctggtgccaaagtggttgcgctcgaaagtgacaaaacttttattccacatttggagtccttaggaaaaaatctggatggaaaactacgagtgattcactgtgacttctttaaactagatcctagaagtggtggagtaataaaaccacctgctatgtcttctcgagggctctttaagaatttgggaatagaagcagttccttggacagcagacatccctttaaaagtagttggaatgttcccaagtagaggtgagaaaagggcactttggaaactcgcatatgacttgtattcctgtacttctatatataaatttggacgaatagaagtaaatatgtttattggtgaaaaagaattccagaaactaatggcagatcccggaaatccagacttgtatcatgtattaagtgttatctggcaattagcttgtgagattaaggttctgcacatggagccttggtcatcatttgatatatacacccggaaagggccgctggaaaacccaaagcgtagggaattattagaccaattacaacaaaagctgtatcttattcaaatgattcctcgtcaaaatttatttaccaagaacttaacacctatgaactataatatattttttcacttgttaaagcactgttttgggaggcgcagcgccactgtaatagaccacttacgttcattgactccacttgatgcgagagatatattgatgcaaataggaaaacaggaggatgagaaagtagttaacatgcaccctcaagacttcaaaacactttttgaaactatagagcgttccaaagattgtgcttataaatggctgtatgatgaaaccctggaagatagg

SEQ ID NO: 12 - protein, 353 aa, Homo sapiens
  Mature TFB2M protein, isoform 1
LSDSSPQLWPEPDFRNPPRKASKASLDFKRYVTDRRLAETLAQIYLGKPSRPPHLLLECNPGPGILTQALLEAGAKVVALESDKTFIPHLESLGKNLDGKLRVIHCDFFKLDPRSGGVIKPPAMSSRGLFKNLGIEAVPWTADIPLKVVGMFPSRGEKRALWKLAYDLYSCTSIYKFGRIEVNMFIGEKEFQKLMADPGNPDLYHVLSVIWQLACEIKVLHMEPWSSFDIYTRKGPLENPKRRELLDQLQQKLYLIQMIPRQNLFTKNLTPMNYNIFFHLLKHCFGRRSATVIDHLRSLTPLDARDILMQIGKQEDEKVVNMHPQDFKTLFETIERSKDCAYKWLYDETLEDR

SEQ ID NO: 13 - DNA, 885 bp, Homo sapiens
  Immature TFB2M coding sequence, isoform X1
atgtggatcccagtggtcgggcttcctcggcggctgaggctctccgccttggcgggcgctggtcgcttttgcattttagggtctgaagcggcgacgcgaaagcatttgccggcgaggaaccactgtgggctctctgactcctctccgcagctgtggcccgaaccggatttcaggaatccgccaaggaaggcgtctaaggccagcttagactttaagcgttacgtaaccgatcggagattggctgagaccctggcgcaaatctatttgggaaaaccaagtagacctccacacctactgctggagtgcaatccaggtcctggaatcctgactcaggcattacttgaagctggtgccaaagtggttgcgctcgaaagtgacaaaacttttattccacatttggagaaactaatggcagatcccggaaatccagacttgtatcatgtattaagtgttatctggcaattagcttgtgagattaaggttctgcacatggagccttggtcatcatttgatatatacacccggaaagggccgctggaaaacccaaagcgtagggaattattagaccaattacaacaaaagctgtatcttattcaaatgattcctcgtcaaaatttatttaccaagaacttaacacctatgaactataatatattttttcacttgttaaagcactgttttgggaggcgcagcgccactgtaatagaccacttacgttcattgactccacttgatgcgagagatatattgatgcaaataggaaaacaggaggatgagaaagtagttaacatgcaccctcaagacttcaaaacactttttgaaactatagagcgttccaaagattgtgcttataaatggctgtatgatgaaaccctggaagatagg

SEQ ID NO: 14 - protein, 295 aa, Homo sapiens
  Immature TFB2M protein, isoform X1
MWIPVVGLPRRLRLSALAGAGRFCILGSEAATRKHLPARNHCGLSDSSPQLWPEPDFRNPPRKASKASLDFKRYVTDRRLAETLAQIYLGKPSRPPHLLLECNPGPGILTQALLEAGAKVVALESDKTFIPHLEKLMADPGNPDLYHVLSVIWQLACEIKVLHMEPWSSFDIYTRKGPLENPKRRELLDQLQQKLYLIQMIPRQNLFTKNLTPMNYNIFFHLLKHCFGRRSATVIDHLRSLTPLDARDILMQIGKQEDEKVVNMHPQDFKTLFETIERSKDCAYKWLYDETLEDR

SEQ ID NO: 15 - DNA, 756 bp, Homo sapiens
  Mature TFB2M coding sequence, isoform X1
ctctctgactcctctccgcagctgtggcccgaaccggatttcaggaatccgccaaggaaggcgtctaaggccagcttagactttaagcgttacgtaaccgatcggagattggctgagaccctggcgcaaatctatttgggaaaaccaagtagacctccacacctactgctggagtgcaatccaggtcctggaatcctgactcaggcattacttgaagctggtgccaaagtggttgcgctcgaaagtgacaaaacttttattccacatttggagaaactaatggcagatcccggaaatccagacttgtatcatgtattaagtgttatctggcaattagcttgtgagattaaggttctgcacatggagccttggtcatcatttgatatatacacccggaaagggccgctggaaaacccaaagcgtagggaattattagaccaattacaacaaaagctgtatcttattcaaatgattcctcgtcaaaatttatttaccaagaacttaacacctatgaactataatatattttttcacttgttaaagcactgttttgggaggcgcagcgccactgtaatagaccacttacgttcattgactccacttgatgcgagagatatattgatgcaaataggaaaacaggaggatgagaaagtagttaacatgcaccctcaagacttcaaaacactttttgaaactatagagcgttccaaagattgtgcttataaatggctgtatgatgaaaccctggaagatagg

SEQ ID NO: 16 - protein, 252 aa, Homo sapiens
  Mature TFB2M protein, isoform X1
LSDSSPQLWPEPDFRNPPRKASKASLDFKRYVTDRRLAETLAQIYLGKPSRPPHLLLECNPGPGILTQALLEAGAKVVALESDKTFIPHLEKLMADPGNPDLYHVLSVIWQLACEIKVLHMEPWSSFDIYTRKGPLENPKRRELLDQLQQKLYLIQMIPRQNLFTKNLTPMNYNIFFHLLKHCFGRRSATVIDHLRSLTPLDARDILMQIGKQEDEKVVNMHPQDFKTLFETIERSKDCAYKWLYDETLEDR

SEQ ID NO: 17 - DNA, 609 bp, Homo sapiens
  Immature TFB2M coding sequence, isoform X2
atgttcccaagtagaggtgagaaaagggcactttggaaactcgcatatgacttgtattcctgtacttctatatataaatttggacgaatagaagtaaatatgtttattggtgaaaaagaattccagaaactaatggcagatcccggaaatccagacttgtatcatgtattaagtgttatctggcaattagcttgtgagattaaggttctgcacatggagccttggtcatcatttgatatatacacccggaaagggccgctggaaaacccaaagcgtagggaattattagaccaattacaacaaaagctgtatcttattcaaatgattcctcgtcaaaatttatttaccaagaacttaacacctatgaactataatatattttttcacttgttaaagcactgttttgggaggcgcagcgccactgtaatagaccacttacgttcattgactccacttgatgcgagagatatattgatgcaaataggaaaacaggaggatgagaaagtagttaacatgcaccctcaagacttcaaaacactttttgaaactatagagcgttccaaagattgtgcttataaatggctgtatgatgaaaccctggaagatagg

SEQ ID NO: 18 - protein, 203 aa, Homo sapiens
  Immature TFB2M protein, isoform X2
MFPSRGEKRALWKLAYDLYSCTSIYKFGRIEVNMFIGEKEFQKLMADPGNPDLYHVLSVIWQLACEIKVLHMEPWSSFDIYTRKGPLENPKRRELLDQLQQKLYLIQMIPRQNLFTKNLTPMNYNIFFHLLKHCFGRRSATVIDHLRSLTPLDARDILMQIGKQEDEKVVNMHPQDFKTLFETIERSKDCAYKWLYDETLEDR

SEQ ID NO: 19 - DNA, 22 bp, Artificial Sequence
  PGC-1alpha (cDNA) forward primer
tgaactgagggacagtgatttc

SEQ ID NO: 20 - DNA, 22 bp, Artificial Sequence
  PGC-1alpha (cDNA) reverse primer
cccaagggtagctcagtttatc

SEQ ID NO: 21 - DNA, 19 bp, Artificial Sequence
  TFAM (cDNA) forward primer
ctcagaacccagatgcaaa

SEQ ID NO: 22 - DNA, 17 bp, Artificial Sequence
  TFAM (cDNA) reverse primer
gccactccgccctataa

SEQ ID NO: 23 - DNA, 21 bp, Artificial Sequence
  Beta actin (cDNA) forward primer
catgtacgttgctatccaggc

SEQ ID NO: 24 - DNA, 21 bp, Artificial Sequence
  Beta actin (cDNA) reverse primer
ctccttaatgtcacgcacgat

SEQ ID NO: 25 - DNA, 20 bp, Artificial Sequence
  Mitochondrial NADH dehydrogenase subunit 1 (cDNA) forward primer
tgggtacaatgaggagtagg

SEQ ID NO: 26 - DNA, 18 bp, Artificial Sequence
  Mitochondrial NADH dehydrogenase subunit 1 (cDNA) reverse primer
ggagtaatccaggtcggt

SEQ ID NO: 27 - DNA, 22 bp, Artificial Sequence
  Ribosomal protein lateral stalk subunit P1 (cDNA) forward primer
agccggtgtaaatgttgagcct

SEQ ID NO: 28 - DNA, 24 bp, Artificial Sequence
  Ribosomal protein lateral stalk subunit P1 (cDNA) reverse primer
tctttgcttccactttcttctcct

SEQ ID NO: 29 - DNA, 20 bp, Artificial Sequence
  Mitochondrial NADH dehydrogenase subunit 1 (DNA) forward primer
cacccaagaacagggtttgt

SEQ ID NO: 30 - DNA, 21 bp, Artificial Sequence
  Mitochondrial NADH dehydrogenase subunit 1 (DNA) reverse primer
tggccatgggtatgttgttaa

SEQ ID NO: 31 - DNA, 20 bp, Artificial Sequence
  18SrRNA (DNA) forward primer
tagagggacaagtggcgttc

SEQ ID NO: 32 - DNA, 18 bp, Artificial Sequence
  18SrRNA (DNA) reverse primer
cgctgagccagtcagtgt

SEQ ID NO: 33 - DNA, 65 bp, Artificial Sequence
  S3 Backbone
ctattagcgtccagtgaatgcgagtccgtctaagagagtagtacagcagccgtcaagagtgtcta

SEQ ID NO: 34 - DNA, 25 bp, Artificial Sequence
  S3 Splint
gttctgtcatatttaagcgtcttaa

SEQ ID NO: 35 - DNA, 48 bp, Artificial Sequence
  Compaction Oligonucleotide
agagagtagtacagcagccgtaaaagagagtagtacagcagccgtuuu

SEQ ID NO: 36 - DNA, 20 bp, Artificial Sequence
  Detection Oligonucleotide
cagtgaatgcgagtccgtct

SEQ ID NO: 37 - DNA, 18 bp, Homo sapiens
  TFAM and TFB2M consensus binding sequence 1
aaagataaaatttgaaat

SEQ ID NO: 38 - DNA, 17 bp, Homo sapiens
  TFAM and TFB2M consensus binding sequence 2
aaagacaccccccacag

SEQ ID NO: 39 - DNA, 126 bp, Homo sapiens
  N-terminal part of immature TFAM coding sequence
atggcgtttctccgaagcatgtggggcgtgctgagtgccctgggaaggtctggagcagagctgtgcaccggctgtggaagtcgactgcgctcccccttcagttttgtgtatttaccgaggtggttt

SEQ ID NO: 40 - protein, 42 aa, Homo sapiens
  N-terminal part of immature TFAM protein
MAFLRSMWGVLSALGRSGAELCTGCGSRLRSPFSFVYLPRWF

SEQ ID NO: 41 - DNA, 129 bp, Homo sapiens
  N-terminal part of immature TFB2M coding sequence
atgtggatcccagtggtcgggcttcctcggcggctgaggctctccgccttggcgggcgctggtcgcttttgcattttagggtctgaagcggcgacgcgaaagcatttgccggcgaggaaccactgtggg

SEQ ID NO: 42 - protein, 43 aa, Homo sapiens
  N-terminal part of immature TFB2M protein
MWIPVVGLPRRLRLSALAGAGRFCILGSEAATRKHLPARNHCG

(19) United States
(12) Patent Application Publication
(10) Pub. No.: US 2025/0271449 A1
(43) Pub. Date: Aug. 28, 2025

(54) USE OF MATURE MITOCHONDRIAL TRANSCRIPTION FACTOR A (TFAM) FOR DIAGNOSING ORGAN DYSFUNCTIONS

(71) Applicant: Universitätsklinikum Knappschaftskrankenhaus Bochum GmbH, Bochum (DE)

(72) Inventors: Tim RAHMEL, Witten (DE); Björn KOOS, Paderborn (DE)

(73) Assignee: Universitätsklinikum Knappschaftskrankenhaus Bochum GmbH, Bochum (DE)

(21) Appl. No.: 18/253,687

(22) PCT Filed: Nov. 19, 2021
(86) PCT No.: PCT/EP2021/082385

§ 371 Date: May 19, 2023

Related U.S. Application Data
(60) Provisional application No. 63/126,703, filed on Dec. 17, 2020.

(30) Foreign Application Priority Data
Nov. 20, 2020 (EP) 20208932.2

(51) Int. Cl. G01N 33/68; C07K 16/18

(52) U.S. Cl. CPC G01N 33/6893; C07K 16/18; G01N 2800/26; G01N 2800/52; G01N 2800/56

(57) ABSTRACT

The present invention relates to in vitro methods for prognosing the outcome of an organ dysfunction in a patient, in vitro methods for diagnosing the degree of severity of an organ dysfunction in a patient and in vitro methods for diagnosing the presence of an organ dysfunction in a subject, wherein said in vitro methods comprise a step of determining the level of mature and/or active mitochondrial transcription factor A (TFAM) protein in a sample. Furthermore, the invention relates to methods of treating a patient in need suffering from an organ dysfunction and methods of treating a patient in need suffering from an infection and/or inflammation. Furthermore, the invention relates to a binding molecule specifically binding the mature and/or active TFAM protein, a binding molecule specifically binding the immature TFAM protein and a kit comprising (i) a primary binding molecule specifically binding TFAM protein and/or (ii) a primary binding molecule specifically binding TFB2M, and to uses of said binding molecules and kit. Furthermore, the invention relates to a method for identifying a compound which promotes the transport of TFAM protein into mitochondria and/or which promotes the maturation of TFAM protein and compounds identified by such a method.

The present invention relates to in vitro methods for prognosing the outcome of an organ dysfunction in a patient, in vitro methods for diagnosing the degree of severity of an organ dysfunction in a patient and in vitro methods for diagnosing the presence of an organ dysfunction in a subject, and in vitro methods for monitoring the course of an organ dysfunction, wherein said in vitro methods comprise a step of determining the level of mature and/or active mitochondrial transcription factor A (TFAM) protein in a sample. Furthermore, the invention relates to methods of treating a patient in need suffering from an organ dysfunction and methods of treating a patient in need suffering from an infection and/or inflammation. Furthermore, the invention relates to a binding molecule specifically binding the mature and/or active TFAM protein, a binding molecule specifically binding the immature TFAM protein and a kit comprising (i) a primary binding molecule specifically binding TFAM protein and/or (ii) a primary binding molecule specifically binding TFB2M, and to uses of said binding molecules and kit. Furthermore, the invention relates to a method for identifying a compound which promotes the transport of TFAM protein into mitochondria and/or which promotes the maturation of TFAM protein and compounds identified by such a method.

Many organ dysfunctions, foremost sepsis, are life-threatening diseases. Sepsis is an acute organ dysfunction caused by a dysregulated immune response to an infection, affecting millions of individuals per year worldwide and representing a major healthcare concern (Singer (2016), JAMA 315; Fleischmann (2016), Am J Respir Crit Care Med 193). Interest has increasingly focused on the link between mitochondrial dysfunction and organ dysfunctions such as sepsis-associated organ failure (Singer (2014), Virulence 5; Arulkumaran (2016), Shock 45; Suomalainen (2018), Nat Rev Mol Cell Biol 19; Kozlov (2017), Redox Biol 13; Maestraggi (2017), Biomed Res Int 2017; Crouser (2004), Mitochondrion 4). Mitochondria generate most of the adenosine triphosphate (ATP) required for normal cellular function, but are also involved in multiple intracellular signaling and regulatory processes such as intracellular calcium regulation and production of reactive oxygen species (McBride (2006), Curr Biol 16; Chan (2006), Cell 125; Galluzzi (2012), Circ Res 111). These important regulatory mechanisms seem to be profoundly disturbed in human sepsis, which can ensue mitochondrial dysfunction and reduced oxidative ATP production (Singer (2014), Virulence 5; Arulkumaran (2016), Shock 45; Singer (2017), Crit Care 21).

Impaired mitochondrial functionality and ability to recover likely contribute to organ dysfunction and death (Singer (2014), Virulence 5; Arulkumaran (2016), Shock 45; Grundler (2014), Crit Care 18; Kraft (2019), Crit Care Med 47). However, mitochondrial dysfunction seems to be highly variable and should not be seen as general denominator for organ dysfunctions such as multiple organ failure in sepsis and septic shock (Jeger (2013), Eur J Clin Invest 43). In fact, the current understanding about mitochondrial dysfunction in sepsis and other organ dysfunctions is still insufficient. Generally, mitochondrial injury and ATP depletion trigger an increased activation of mitochondrial biogenesis, aimed to ameliorate the cellular effects of mitochondrial dysfunction (Kunkel (2016), Heart Fail Rev 21; Gureev (2019), Front Genet 10; Cherry (2015), Antioxid Redox Signal 22). It is thought that the activation of the mitochondrial biogenesis is mediated by a signaling network that promotes the expression of the nuclear-encoded mitochondrial transcription factor A (TFAM) (Kunkel (2016), Heart Fail Rev 21). TFAM plays a central role in the mitochondrial core transcription initiation complex (Ramachandran (2017), Nucleic Acids Res 45) that is required not only for expression of mitochondrial-encoded respiratory chain subunits but also for mitochondrial DNA replication (Hillen (2017), Cell 171; Agaronyan (2015), Science 347). TFAM regulates de novo synthesis of mitochondrial proteins, facilitates mitochondrial DNA replication, and mediates mitochondrial DNA protection (Campbell (2012), Biochim Biophys Acta 1819; Kasashima (2011), Exp Cell Res 317). Furthermore, lack of TFAM may entail mitochondrial dysfunction and an energy crisis, with insufficient TFAM resulting in possible death (Stiles (2016), Mol Genet Metab 119).

However, recent studies, provide growing evidence that activation of mitochondrial biogenesis in sepsis, although associated with an increased intracellular TFAM expression, is not necessarily accompanied by recovery of mitochondrial function (Kraft (2019), Crit Care Med 47; Vanasco (2014), Free Radic Biol Med 77; Suliman (2004), Cardiovasc Res 64; Hickson-Bick (2008), J Mol Cell Cardiol 44). Yet, it is still not clear why the mitochondrial dysfunction and associated organ dysfunction persists although the expression of TFAM—a major promoter of mitochondrial biogenesis—is increased. In particular, it is still mysterious what implications TFAM exactly has for organ dysfunctions such as sepsis, or whether it has any at all.

To date, the diagnosis of organ dysfunctions such as sepsis mainly relies on time-consuming in vivo measurements such as the SOFA score (Singer (2016), JAMA 315). However, sepsis or other organ dysfunctions are life-threatening conditions that need to be rapidly diagnosed, preferably by point-of-care devices that can be operated by non-professionals. Furthermore, the treatment of septic patients is currently rather unspecific and limited, and often focused on generic means such as the stabilization of the patients in an intensive care unit (ICU), and the administration of drugs which often do not have any benefit (Fujii (2020), JAMA; 323(5)).

The complex diagnosis and limited treatment options for organ dysfunctions such as sepsis are in sharp contrast to their high incidence and mortality rate.

In an earlier report (Kraft (2019), Crit Care Med 47, it has been observed that TFAM mRNA levels were slightly lower at day 1 in septic patients which were not ICU-free after one week. However, the variation of the TFAM data was rather high. Furthermore, it was observed by Kraft et al. that TFAM mRNA levels increased within the next two or four days, and that at day 3 or 5, TFAM mRNA levels were not associated with ICU-freedom. Moreover, Kraft et al. further reports that TFAM mRNA were not associated at all with 90-day mortality, the APACHE II score, or the SOFA score (i.e. the most commonly used indicator of the severity of sepsis). Thus, the gene expression data in Kraft et al. suggest that TFAM is not a reliable and robust biomarker for sepsis diagnosis and is neither indicative of the degree of severity of sepsis nor predictive of the survival of septic patients. Moreover, the data in Kraft et al. indicate that reliance on TFAM mRNA as a diagnostic marker may lead to wrong and potentially fatal therapeutic decisions, e.g. premature discharge of a patient still suffering from sepsis from the intensive care unit.

Thus, there is still a need for better means and methods for diagnosing, and consequently treating, subjects suffering from an organ dysfunction and/or subjects suspected of suffering from an organ dysfunction.

The above technical problem is solved by the embodiments as defined in the claims and as characterized in the embodiments provided herein.

Accordingly, the invention relates to an in vitro method for diagnosing the degree of severity of an organ dysfunction in a patient, wherein said method comprises the steps of
(a) determining the level of mature and/or active mitochondrial transcription factor A (TFAM) protein in a sample from said patient; and
(b) determining the degree of severity of the organ dysfunction based on the level of mature and/or active TFAM, wherein the degree of severity is negatively correlated with the level of mature and/or active TFAM protein.

Furthermore, the invention relates to an in vitro method for prognosing the outcome of an organ dysfunction in a patient, wherein said method comprises the steps of
(a) determining the level of mature and/or active mitochondrial transcription factor A (TFAM) protein in a sample from said patient;
(b) comparing the level of mature and/or active TFAM protein to a reference level; and
(c) prognosing the outcome of said organ dysfunction, wherein
   (i) a positive outcome is prognosed when the level of mature and/or active TFAM protein is equal to or higher than said reference level, and/or
   (ii) a negative outcome is prognosed when the level of mature and/or active TFAM protein is lower than said reference level.

The invention is, at least partly, based on the surprising discovery that the amount of mature TFAM protein (˜24 kDa) in the mitochondria was significantly reduced in septic patients while the amount of TFAM mRNA and immature TFAM protein (˜29 kDa; pre-TFAM) outside mitochondria (cytonucleoplasm) was significantly increased (FIG. 7).

Moreover, the inventors surprisingly found, as further illustrated in the appended Examples, that the amount of mature TFAM protein (˜24 kDa) in the mitochondria was associated with the survival of septic patients, whereas the amount of immature TFAM protein (˜29 kDa) in the cytonucleoplasm was not associated with the survival of such patients (FIG. 10). Although, the association of mature mitochondrial TFAM protein (as determined by western blot) with the survival of the septic patients was, as such, not statistically significant (probably due to the relatively few data points), it was nonetheless striking that above a certain threshold all (4/4) septic patients have survived for at least 30 days, whereas below that threshold two third ( 4/6) of the septic patients have died within 30 days (FIG. 10b).

Furthermore, septic patients with a low SOFA score (10 or lower) had a higher amount of mature mitochondrial TFAM protein than septic patients with a SOFA score above 10 (FIG. 10a).

Further unexpectedly, the inventors found that the mitochondria contained much more immature TFAM protein (˜29 kDa) than mature TFAM protein (˜24 kDa) (FIG. 3c). Thus, the amount of TFAM protein in the mitochondria, e.g. as determined by immunofluorescence with an anti-TFAM antibody, is not associated with the survival of the septic patients. This is further corroborated by the finding that the amount of the immature TFAM protein in the mitochondria did not change upon stimulation of healthy cells with LPS as determined by Western blot (see FIG. 3c, the thick bands above the 24 kDa bands).

Further surprisingly, the inventors found that the ratio of the amount of mature mitochondrial TFAM protein (˜24 kDa) over the amount of immature cytonucleoplasmic TFAM protein (˜29 kDa) had a great statistical power, i.e. a high sensitivity and specificity, for prognosing the 30-days survival of septic patients, wherein the area under the curve of the ROC plot was about 0.88 (FIG. 11).

It was thus surprisingly found by the inventors that only the mature TFAM protein or the ratio of mature TFAM protein over immature TFAM protein, but not the TFAM mRNA, the immature TFAM protein or the localization of the TFAM protein, may be used as a marker for diagnosing the presence, severity and/or outcome of an organ dysfunction such as sepsis.

Furthermore, the inventors surprisingly found, as illustrated in the appended Examples, that the cells of septic patients showed significantly less interactions of TFAM protein with TFB2M protein than cells of healthy control subjects (FIG. 9).

Without being bound by theory, only the mature TFAM protein (˜24 kDa, but not the immature TFAM protein (˜29 kDa) binds to TFB2M protein, i.e. the mature TFB2M protein, within the mitochondrial core transcription initiation complex (Hillen (2017, Cell 171). Moreover, the binding of TFAM protein to TFB2M protein is most likely necessary and sufficient for the TFAM protein to be active. Thus, the terms mature TFAM protein and active TFAM protein have a very similar meaning and are often used interchangeably herein, wherein the term “mature TFAM protein” primarily refers to the size and/or sequence of the TFAM protein and the “active TFAM protein” primarily refers to the interaction of TFAM protein with TFB2M protein, i.e. the TFAM-TFB2M complex.

Particularly surprisingly, the amount of interactions of TFAM protein with TFB2M protein (TFAM-TFB2M complex) was significantly associated with the survival of septic patients (FIG. 12). Strikingly, above a certain threshold of TFAM-TFB2M protein interactions, all (5/5) septic patients survived for at least 30 days, whereas below said threshold 80% (⅘) of the septic patients died within 30 days.

This demonstrates that the number/amount of interactions of TFAM protein with TFB2M protein is a particularly effective indicator for prognosing the outcome of an organ dysfunction such as sepsis. Furthermore, the inventors surprisingly found that the number of interactions of TFAM protein with TFB2M protein showed a strong inverse correlation with the SOFA score of the septic patients (r=−0.764; FIG. 12). This demonstrates that the number/amount of interactions of TFAM protein with TFB2M protein is also a particularly effective indicator for diagnosing the severity of an organ dysfunction such as sepsis.

Furthermore, the inventors surprisingly found that the outcome of sepsis, i.e. the recovery from sepsis, e.g. as demonstrated by discharge from an intensive care unit (ICU freedom), or the patient survival is linked to the severity of sepsis at an earlier time-point as determined by the number/amount of interactions of TFAM protein with TFB2M protein (FIGS. 15 and 16). This corroborates the notion that the number/amount of interactions of TFAM protein with TFB2M protein is a reliable and robust indicator of the degree of severity of an organ dysfunction such as sepsis, and that the severity of the organ dysfunction determined accordingly, is associated with the outcome of the organ dysfunction.

Further analyses showed that TFAM mRNA levels had much less informative value and were much less robust and reliable than mature and/or active TFAM protein levels. In particular, the variation of the TFAM mRNA data was considerably higher, and TFAM mRNA data could (in contrast to TFAM-TFB2M interactions) not predict ICU-freedom (FIG. 17). It was thus further found in the context of the present invention that the mature and/or active TFAM protein is an improved biomarker compared to TFAM mRNA for diagnosing and/or prognosing the presence, severity and/or outcome of an organ dysfunction such as sepsis. Yet, the inventors unexpectedly further found that an increased TFAM mRNA level was associated with death of the patients within one month (FIG. 17, bottom panel), although the association was less pronounced, less significant and less robust than the association observed between a decreased number of TFAM-TFB2M protein-protein interactions and death of the patients (FIG. 15, bottom panel).

In addition, the inventors surprisingly found that serial (repeated) measurement of the number/amount of interactions of TFAM protein with TFB2M protein in septic patients allows to monitor and/or predict the course (i.e. progression or regression) of the sepsis, e.g. the recovery from the sepsis (FIG. 16). Thus, measuring the amount of active and/or mature TFAM protein, in particular repeatedly over time (e.g. every 24 h or 28 h), can be advantageously used for monitoring the treatment success and/or adjusting the treatment regime in patients suffering from an organ dysfunction such as sepsis, e.g. by administering or withdrawing administration of a supportive drug, as described herein.

Furthermore, although an in vitro inflammation model would certainly not be sufficient for making any conclusions about the suitability of a marker for diagnosing the presence of an organ dysfunction such as sepsis, let alone the severity and/or outcome of such an organ dysfunction, it was still another remarkable finding that the amount of mature mitochondrial TFAM protein and the number of protein interactions of TFAM with TFB2M decreased upon challenging healthy cells with lipopolysaccharide (LPS), while the amount of TFAM mRNA and immature cytonucleoplasmic TFAM protein respectively increased (FIGS. 1 and 5).

Hence, the inventors surprisingly found that the mature and/or active TFAM protein can be used as a marker for diagnosing and/or prognosing the presence, severity and/or outcome of an organ dysfunction such as sepsis.

Thus, the invention further relates to an in vitro method for prognosing the outcome of an organ dysfunction in a patient, wherein said method comprises the steps of
(a) determining the level of mature and/or active mitochondrial transcription factor A (TFAM) protein in a sample from said patient;
(b) comparing the level of mature and/or active TFAM protein to a reference level; and
(c) prognosing the outcome of said organ dysfunction, wherein
   (i) the chance of a favorable outcome is high when the level of mature and/or active TFAM protein is at least as high as the reference level;
   (ii) the chance of a favorable outcome is low when the level of mature and/or active TFAM protein is below the reference level;
   (iii) the chance of an unfavorable outcome is high when the level of mature and/or active TFAM protein is below the reference level; and/or
   (iv) the chance of an unfavorable outcome is low when the level of mature and/or active TFAM protein is at least as high as the reference level.

In the context of the invention, the level of mature and/or active TFAM protein may correspond to
(i) the amount and/or concentration of mature TFAM protein, preferably the amount of mature TFAM protein,
(ii) the number of interactions of TFAM protein with at least one other mitochondrial protein and/or mitochondrial DNA to which the mature TFAM protein binds specifically, and/or
(iii) the ratio of the amount of mature TFAM protein over the amount of immature TFAM protein.

In some embodiments, the level of mature and/or active TFAM protein corresponds to the amount of mature TFAM protein normalized by the amount of TNF receptor associated protein 1 (TRAP1), in particular the amount of TRAP1 protein in the mitochondria.

Expressions such as X “normalized by” Y and X “normalized to” Y are used interchangeably herein and refer, in particular, to the ratio X over Y (=X/Y).

In preferred embodiments, the level of mature and/or active TFAM protein corresponds to the number of interactions of TFAM protein with mitochondrial transcription factor B2 (TFB2M) protein. The terms “number” and “amount” of interactions of TFAM protein with TFB2M protein are used interchangeably herein. In particular, the number of said interactions may correspond to the amount of the TFAM-TFBM2 complex which may be determined, inter alia, by a proximity ligation assay (PLA) and/or a proximity-dependent initiation of hybridization chain reaction (proxHCR) as described herein. Advantageously, determining the amount of active TFAM protein in mitochondria by determining the number of TFAM-TFB2M protein interactions does not require isolating mitochondria in vitro or in silico, because the TFAM-TFB2M complex occurs inherently only in mitochondria.

In the context of the present invention, the organ dysfunction may be associated with and/or caused by a mitochondrial dysfunction, such as, inter alia, acute heart failure, acute kidney injury, delirium, acute respiratory failure (e.g. acute respiratory distress syndrome; ARDS), and/or acute liver failure. Accordingly, the patient to be diagnosed may show a mitochondrial dysfunction. The mitochondrial dysfunction may be characterized by a reduced oxidative ATP production and/or an altered oxygen consumption rate. For example, the basal respiration, coupled respiration and/or spare respiratory capacity may be reduced, when a mitochondrial dysfunction is present. However, when the mitochondrial dysfunction is associated with a mitochondrial state 4 respiration characterized by uncoupling and/or leaking, the oxygen consumption rate may be increased.

The term “patient to be diagnosed”, as used herein, may refer to the prognostic and/or diagnostic methods provided herein, e.g. to a patient for whom the outcome of its organ dysfunction is prognosed and/or for whom the degree of severity of its organ dysfunction is diagnosed.

In the context of the present invention, the patient or subject may be a mammal such as, inter alia, a human, a horse, dog, cat, cow, pig, goat, sheep, mouse, rat, guinea pig, rabbit, camel, alpaca, or monkey.

Preferably herein, and in the context of the present invention, the patient or subject is a human.

Further in the context of the present invention, the organ dysfunction may be associated with and/or caused by an infection. Accordingly, the patient to be diagnosed may be suffering from an infection.

An infection, as used herein, may be an infection by a pathogenic bacterium such as, inter alia, Escherichia coli, Pseudomonas aeruginosas, Klebsiella pneumoniae, Streptococcus pneumoniae and/or Staphylococcus aureus, preferably Escherichia coli and/or Staphylococcus aureus; a pathogenic virus such as, inter alia, severe acute respiratory syndrome coronavirus-2 (SARS-CoV-2), Herpes simplex virus, Influenza virus and/or Varicella zoster virus, preferably SARS-CoV-2; a pathogenic fungus such as, inter alia, Candida albicans; and/or another pathogenic microorganism such as, inter alia, an amoeba, a plasmodium, and/or a trypanosome.

In a preferred embodiment of the invention, the organ dysfunction is sepsis. Sepsis may be defined as in Singer (2016), JAMA 315. Furthermore, sepsis may comprise a septic shock. For example, the sepsis may be pediatric sepsis and/or the patient may be a child. Furthermore, the sepsis according to the invention may be associated with and/or caused by a disease that is associated with and/or caused by a pathogenic bacterium, virus, fungus or other microorganism as described herein. For example, the sepsis may be associated with and/or caused by COVID19, a disease that is caused and/or associated with SARS-CoV-2.

Hence, the organ dysfunction and/or the status of the patient may be characterized by a SOFA score and/or a quick SOFA score (qSOFA score) of at least 2. In particular, as used herein, the SOFA score and the qSOFA score refer to Singer (2016), JAMA 315, and Vincent (1996), Intensive Care Med 22). Furthermore, the SOFA score, as used herein, is on a scale of 0 to 24 points, wherein more points indicate a more severe organ dysfunction. In particular, in the context of the invention, the organ dysfunction and/or patient status may be characterized by a SOFA score of at least 2. Furthermore, the patient may suffer from an organ dysfunction when the SOFA score of said patient has increased by at least 2 points compared to an earlier timepoint (e.g. during the stay in a hospital and/or during the transfer into a hospital).

Furthermore, in the context of the present invention, the organ dysfunction may be associated with and/or caused by an inflammation. Accordingly, the patient may be suffering from an inflammation.

The inflammation, as used herein, may be associated with and/or caused by, for example, an infection as described herein, and/or by an autoimmune reaction such as an allergic reaction and/or an anaphylactic shock, and/or an autoimmune disease such, inter alia, multiple sclerosis, systemic lupus erythematosus, Myasthenia Gravis and/or Kawasaki disease

Furthermore, the patient may fulfill at least two criteria of the systemic inflammatory response syndrome (SIRS). The four SIRS criteria are defined as: (1) fever >38.0° C. or hypothermia <36.0° C., (2) tachycardia >90 beats/minute, (3) tachypnea >20 breaths/minute, and (4) leucocytosis >12*109/I or leucopoenia <4*109/I.

Furthermore, the patient to be diagnosed may have an increased level of TNF-α, IL-6, IL-10, PGC-1α, TFAM mRNA, and/or total, immature and/or cytonucleoplasmic TFAM protein, in particular compared to a healthy subject of the same species that is not suffering or recovering from an inflammation and/or infection, e.g. as illustrated in the appended Examples.

In particular, the organ dysfunction may be associated with and/or caused by a dysregulated immune response to an infection. Accordingly, the patient may show a dysregulated immune response to an infection. In particular, the dysregulated immune response to an infection may be characterized by an excessive production of TNF-α, IL-6, IL-10, and/or IL-2.

Thus, the organ dysfunction may be an acute organ dysfunction. Furthermore, the organ dysfunction may be life-threatening and/or require an immediate medical intervention.

Furthermore, the organ dysfunction may affect at least 2, preferably at least 4, preferably at least 6 organs. For example, the organ dysfunction may comprise an acute kidney injury. Thus, the organ dysfunction may be a sepsis-related organ failure and/or a multiple organ failure.

Furthermore, the patient to be diagnosed may suffer from acute respiratory distress syndrome, intensive care unit (ICU) acquired weakness and/or a post intensive care syndrome such as a post-operative cognitive dysfunction, a myopathy, and/or a neuropathy.

In the context of the invention, e.g. in the context of the prognostic method of the invention, the positive outcome may comprise (i) survival for at least one month, (ii) in-hospital survival, (iii) health-related quality of life after at least one month, (iv) recovery from the organ dysfunction, e.g. within one month, (v) amelioration of the organ dysfunction, e.g. within one month, and/or (vi) no aggravation of the organ dysfunction.

Preferably, the recovery from the organ dysfunction comprises discharge from an intensive care unit (ICU) or, in other words, “ICU freedom”, e.g. within one week.

Furthermore, the negative outcome may comprise (i) death within one month, (ii) in-hospital death, (iii) a requirement for continuing observation of the organ function and/or stay in a hospital, preferably in an intensive care unit, e.g. for longer than a month, (iv) nursing care-dependency, e.g. for longer than a month, (v) persistence of the organ dysfunction, e.g. for longer than a month, and/or (vi) aggravation of the organ dysfunction.

Preferably, the persistence or aggravation of the organ dysfunction comprises stay in an intensive care unit (ICU), or, in other words, “No ICU freedom”, e.g. for at least one week.

In particular, herein, the start of a time span such as death or survival within one month, or ICU freedom or no ICU freedom within one week is at the day of the diagnosis of sepsis (e.g. by using the SOFA score) which may be considered “day 1”.

In particular, the in-hospital survival or in-hospital death describes whether a patient that is in a hospital (or comparable medical care site) because of an organ dysfunction (e.g. sepsis) leaves the hospital (or another hospital/medical site to which he or she was transferred) alive or dead, respectively.

In particular, a health-related quality of life describes a physiological and psychological state which is at least as good as before the onset of the organ dysfunction (e.g. sepsis), e.g. that the patient is not dependent on nursing care and/or does not have to stay in an intensive care unit. Similarly, the recovery from the organ dysfunction may be a full recovery to a state which is at least as good as before the onset of the organ dysfunction. A corresponding negative outcome may be that the patient requires continuing observation of the organ function and/or has to stay in a hospital, e.g. in an intensive care unit, e.g. for longer than a month and/or is dependent on nursing care, e.g. for longer than a month.

Preferably, the positive outcome comprises at least survival for at least one month and/or ICU freedom within one week, and/or the negative outcome comprises at least death within one month and/or no ICU freedom for at least one week.

In the context of the present invention, the reference level may be determined by analyzing the level of mature and/or active TFAM protein in samples from a plurality of reference patients diagnosed with an organ dysfunction at various degrees of severity, wherein it is known whether the outcome of the organ dysfunction of said reference patients has been positive or negative. Preferably, the level of mature and/or active TFAM protein in the samples from said reference patients is determined by the same measurement method that is employed in step (a) of the inventive diagnostic and/or prognostic methods provided herein. Preferably, the organ dysfunction of the reference patients is the same type of organ dysfunction from which the patient to be diagnosed is suffering from, or from which the patient to be diagnosed is suspected of suffering. It is known in the art that the absolute level of a biomarker (herein mature and/or active TFAM protein) may depend on the measurement method. However, the relationship between two variables X (e.g. the mature and/or active TFAM level) and Y (e.g. the outcome and/or severity of the organ dysfunction) may be robust, independent of variations in the measurement method. For example, when multiple samples (e.g. from reference patients) with known properties (e.g. the outcome or severity of the organ dysfunction) are measured with a certain measurement method and graphed, then the same properties (e.g. the outcome or severity of the organ dysfunction) can be determined for an unknown sample (e.g. from the subject or patient to be diagnosed) that is measured with the same measurement, e.g. by interpolation of the graph.

Furthermore, a threshold (i.e. the reference level) may be determined based on the data from the reference patients, wherein said threshold allows to separate the reference patients with a positive outcome from those with a negative outcome in a useful and/or optimal way.

Thus, reference patients allow to establish a standard curve and/or a threshold (i.e. the reference level) for different data sets and/or in different laboratories.

In particular, the reference level may be selected such that
(i) the level of mature and/or active TFAM protein in the samples of at least about 60%, 70%, 80%, 90%, or at least about 95%, 97% or 99%, e.g. at least about 80%, of the reference patients with a positive outcome is equal to or higher than said reference level, and/or
(ii) the level of mature and/or active TFAM protein in the samples of at least about 60%, 70%, 80%, 90%, or at least about 95%, 97% or 99%, e.g. at least about 80%, of the reference patients with a negative outcome is lower than said reference level.

In the context of the inventive diagnostic and/or prognostic methods provided herein, the power of the prognosis (i.e. the statistical power) may characterized in that
(I) the area under the curve (AUC) of the receiver operating characteristic curve (ROC curve) is at least about 0.6, 0.7, 0.8 or 0.9, preferably at least about 0.7, e.g. at least about 0.68, more preferably at least about 0.8, e.g. at least about 0.88; and/or
(II) (i) the true positive rate is at least about 30%, preferably at least about 50%, and the false positive rate is at most about 15%, preferably at most about 5%,
   (ii) the true positive rate is at least about 50%, preferably at least about 70% and the false positive rate is at most about 30%, preferably at most about 20%, or
   (iii) the true positive rate is at least about 70%, preferably at least about 90% and the false positive rate is at most about 50%, preferably at most about 40%.

Surprisingly, as further illustrated in the appended Examples, such a high statistical power may be achieved by using only a single biomarker, e.g. (i) the mature and/or active TFAM level and/or (ii) the ratio of the amount of mature mitochondrial TFAM protein over the amount of immature cytonucleoplasmic TFAM protein. It is likely, as illustrated in the appended Examples, that using the number of TFAM-TFB2M interactions as biomarker provides an at least as good or even greater statistical power than using the ratio of the amount of mature mitochondrial TFAM protein over the amount of immature cytonucleoplasmic TFAM protein.

Furthermore, the level of mature and/or active TFAM may be used in combination with other biomarkers or indicators of an organ dysfunction, and machine learning techniques may be employed for prognosing that the organ dysfunction has a negative or positive outcome, and/or for diagnosing whether the organ dysfunction is very severe or not. It is well possible that this further enhances the statistical power of the diagnostic and/or prognostic methods provided herein.

In some embodiments of the invention, the level of mature and/or active TFAM may be used in combination with the level of TFAM mRNA for diagnosing the degree of severity of an organ dysfunction and/or for prognosing the outcome of an organ dysfunction, as described herein in the context of the present invention.

For example, the in vitro method for prognosing the outcome of an organ dysfunction in a patient according to the invention may further comprise,
in step (a): determining the level of TFAM mRNA in the sample;
in step (b): comparing the level of TFAM mRNA to a corresponding reference level; and
in step (c): prognosing the outcome of said organ dysfunction, wherein
   (i) a positive outcome, e.g. survival for at least one month, is prognosed when the level of TFAM mRNA is lower than the corresponding reference level,
   and/or
   (ii) a negative outcome, e.g. death within one month, is prognosed when the level of TFAM mRNA is higher than said corresponding reference level.

Notably, the reference level for TFAM mRNA is independent from the reference level for mature and/or active TFAM protein. However, the corresponding reference level may be determined in an analogous manner, as described herein for the mature and/or active TFAM protein.

In some aspects, the invention relates to an in vitro method for prognosing the outcome of an organ dysfunction in a patient, wherein said method comprises the steps of
(a) determining the level of TFAM mRNA in a sample from said patient;
(b) comparing the level of TFAM mRNA to a reference level; and
(c) prognosing the outcome of said organ dysfunction, wherein
(i) a positive outcome, e.g. survival for at least one month, is prognosed when the level of TFAM mRNA is lower than said reference level,
and/or
(ii) a negative outcome, e.g. death within one month, is prognosed when the level of TFAM mRNA is equal to or higher than said reference level.

However, the aspects of the invention which comprise determining the level of mature and/or active TFAM protein in a sample from a patient or which are based on the determination of mature and/or active TFAM protein are more preferred than the aspects which are solely or primarily based on determination of the TFAM mRNA level.

Furthermore, the invention relates to an in vitro method for diagnosing the degree of severity of an organ dysfunction in a patient, wherein said method comprises the steps of
(a) determining the level of mature and/or active mitochondrial transcription factor A (TFAM) protein in a sample from said patient; and
(b) determining the degree of severity of the organ dysfunction based on the level of mature and/or active TFAM, wherein the degree of severity is negatively correlated with the level of mature and/or active TFAM protein.

In particular, a high level of mature and/or active TFAM corresponds to a low degree of severity and a low level of mature and/or active TFAM corresponds to a high degree of severity (very severe organ dysfunction).

The terms “negatively correlated” and “inversely correlated” are used interchangeably herein.

Reference patients and/or standard curves may be employed for determining the severity of the organ dysfunction, even in a gradual manner, as described herein, e.g. in the context of the prognostic method of the invention. Furthermore, a threshold (reference level) may be determined for classifying the organ dysfunction as very severe (i.e. high degree of severity) or not very severe (i.e. low degree of severity), as described herein, e.g. in the context of the prognostic method of the invention.

Furthermore, the in vitro method for diagnosing the degree of severity of an organ dysfunction in a patient may further comprise in said step (a) determining the level of TFAM mRNA in the sample, wherein in said step (b) determining the degree of severity of the organ dysfunction may be further based on the level of TFAM mRNA, and wherein the degree of severity is positively correlated with the level of TFAM mRNA. In particular, a high level of TFAM mRNA corresponds to a high degree of severity (very severe organ dysfunction) and a low level of TFAM mRNA corresponds to a low degree of severity.

Furthermore, the method for diagnosing the degree of severity of an organ dysfunction according to the invention may further comprise the following step (a′) prior to step (b):
(a′) comparing the level of mature and/or active TFAM protein to a reference level, and wherein in step (b) it is determined that
   (i) the degree of severity is low when the level of mature and/or active TFAM protein is equal to or higher than said reference level, and/or
   (ii) the degree of severity is high when the level of mature and/or active TFAM protein is lower than said reference level.

In particular, the reference level may correspond to the reference level of the prognostic method of the invention. Furthermore, the predictive power of the diagnosis may correspond to the predictive power of the prognostic method of the invention.

In some embodiments, said step (a′) may further comprise comparing the level of TFAM mRNA to a corresponding reference level. In such embodiments, it may be further confirmed in step that the degree of severity is low, when the level of TFAM mRNA is lower than the corresponding reference level, and/or that the degree of severity is high when the level of TFAM mRNA is higher than said reference level.

In particular, the degree of severity of the organ dysfunction, as used herein, corresponds to the SOFA score. For example, a low degree of severity may correspond to a SOFA score of at most 10, and/or a high degree of severity may correspond to a SOFA score of greater than 10.

Thus, the reference level, in the context of the diagnostic and/or prognostic methods of the invention, may correspond to a SOFA score of 10, 11 or 12, preferably 10 or 11, more preferably 10.

Furthermore, the invention relates to an in vitro method for monitoring the course of an organ dysfunction such as sepsis in a patient, wherein said method comprises carrying out repeatedly the following step (a):
(a) determining the level of mature and/or active mitochondrial transcription factor A (TFAM) protein in a sample from said patient, as described herein,
   and at least once the following step (b):
(b) determining the course of the sepsis in the patient, wherein
   (i) recovery from the organ dysfunction is diagnosed when the level of mature and/or active TFAM protein has increased over time,
   (ii) persistence of the organ dysfunction is diagnosed when the level of mature and/or active TFAM protein remained similar over time, and/or
   (iii) aggravation of the organ dysfunction is diagnosed when the level of mature and/or active TFAM protein has decreased over time.

In particular, said step (a) may performed at least twice or three times and/or every 24 h to 48 h, preferably every 24 h, e.g. for at least 2 or 3 consecutive days.

In particular, in said case (i), the level of mature and/or active TFAM protein may have increased by at least 10% within one day, or at least 10% per day, e.g. for at least three consecutive days, in said case (ii), the level of mature and/or active TFAM protein may have not changed by more than 10% within 1, 2 and/or 3 days; and/or in said case (iii), the level of mature and/or active TFAM protein may have decreased by at least 10% within one day, or at least 10% per day, e.g. for at least three consecutive days. An increase (e.g. at least 10% increase per day) of the level of mature and/or active TFAM protein over time, e.g. within one, two, three days or one or two weeks, may allow to predict that the patient will be ICU-free within one week, and/or survive for at least one month.

Furthermore, no significant change (e.g. overall ±10%) or, even more so, a decrease (e.g. at least 10% decrease per day) of the level of mature and/or active TFAM protein over time, e.g. within one, two, three days or one or two weeks, may allow to predict that the patient will not be ICU-free within one week, and/or die within one month.

It is thought that, after cytosolic synthesis as an immature precursor protein (˜29 kDa), TFAM is shuttled to the mitochondria, crossing the outer and inner membranes. The mature TFAM (˜24 kDa) is then generated by cleavage of a targeting sequence (˜5 kDa) by a processing peptidase in the mitochondrial matrix (Garstka (2003), Nucleic Acids Res 31; Prasai (2017), Pathophysiology 24).

Thus, as used herein, the mature and/or active TFAM protein has usually a molecular mass of about 22 to 26 kDa, preferably about 24 kDa. As described above, although the terms “mature TFAM protein” and “active TFAM protein” have a very similar meaning and may be used interchangeably herein, said molecular mass refers, in particular, to the mature TFAM protein.

Furthermore, as used herein, the immature TFAM protein has usually a molecular mass of about 27 to 30 kDa, preferably about 29 kDa. The immature TFAM protein may be also considered “inactive”, as described above, e.g. because it does not bind TFB2M protein within the mitochondrial core transcription initiation complex. Furthermore, the immature TFAM protein may be also considered “inactive” because it is not at the location where TFAM needs to be for being active (i.e. in the mitochondrial matrix and/or at the mitochondrial genome).

As used herein and in the context of the present invention, the mature and/or active TFAM protein, in particular the mature TFAM protein, may refer to:
(i) a human mature TFAM protein with a sequence set forth in SEQ ID NO:4 or SEQ ID NO:8, preferably SEQ ID NO:4;
(ii) a protein that is encoded by a nucleotide sequence set forth in SEQ ID NO:3 or SEQ ID NO:7, preferably SEQ ID NO:3;
(iii) a mature TFAM protein that is orthologous to the human mature TFAM protein of (i);
(iv) a protein that is encoded by a nucleotide sequence which is orthologous to the nucleotide sequence described in (ii); and/or
(v) a protein that has at least 60%, 70%, 80% or 90%, preferably at least 90%, 95%, 98% or 99%, e.g. 95%, 96%, 97%, 98%, or 99% sequence identity to the protein of any one of (i) to (iv), preferably (i) or (ii), more preferably (i).

In particular, the mature TFAM protein may be a human mature TFAM protein with a sequence set forth in SEQ ID NO:4 or SEQ ID NO:8, preferably SEQ ID NO:4, and/or a protein that has at least 60%, 70%, 80% or 90%, preferably at least 90%, 95%, 98% or 99%, e.g. 95%, 96%, 97%, 98%, or 99% sequence identity to said human mature TFAM protein. Preferably, the mature TFAM protein has a sequence set forth in SEQ ID NO:4 or SEQ ID NO:8, preferably SEQ ID NO:4.

As used herein and in the context of the present invention, the immature TFAM protein, may refer to:
(i) a human immature TFAM protein with a sequence set forth in SEQ ID NO:2 or SEQ ID NO:6, preferably SEQ ID NO:2;
(ii) a protein that is encoded by a nucleotide sequence set forth in SEQ ID NO:1 or SEQ ID NO:5, preferably SEQ ID NO:1;
(iii) a mature TFAM protein that is orthologous to the human immature TFAM protein of (i);
(iv) a protein that is encoded by a nucleotide sequence which is orthologous to the nucleotide sequence described in (ii); and/or
(v) a protein that has at least 60%, 70%, 80% or 90%, preferably at least 90%, 95%, 98% or 99%, e.g. 95%, 96%, 97%, 98%, or 99% sequence identity to the protein of any one of (i) to (iv), preferably (i) or (ii), more preferably (i).

In particular, the immature TFAM protein may be a human immature TFAM protein with a sequence set forth in SEQ ID NO:2 or SEQ ID NO:6, preferably SEQ ID NO:2, and/or a protein that has at least 60%, 70%, 80% or 90%, preferably at least 90%, 95%, 98% or 99%, e.g. 95%, 96%, 97%, 98%, or 99% sequence identity to said human immature TFAM protein. Preferably, the immature TFAM protein has a sequence set forth in SEQ ID NO:2 or SEQ ID NO:6, preferably SEQ ID NO:2.

In particular, the immature TFAM protein, as used herein, contains a N-terminal part that is cleaved off when the mature TFAM protein is generated.

Accordingly, the mature TFAM protein, as used herein, does, in particular, not contain, i.e. not at the N-terminal end, a sequence corresponding to the N-terminal part of the immature TFAM protein that is cleaved off when the mature TFAM protein is generated. For example, said N-terminal part of the immature TFAM protein may have the amino acid sequence set forth in positions 1 to 42 of SEQ ID NO:2 (i.e. as set forth in SEQ ID NO: 40), an amino acid sequence encoded by the nucleic acid sequence set forth in positions 1 to 126 of SEQ ID NO:1 (i.e. as set forth in SEQ ID NO: 39), or an amino acid sequence that has at least 60%, 70%, 80% or 90%, preferably at least 90%, 95%, 98% or 99%, e.g. 95%, 96%, 97%, 98%, or 99% sequence identity to any of said amino acid sequences.

As used herein and in the context of the present invention, the mature and/or active TFB2M protein, in particular the mature TFB2M protein, may refer to:
(i) a human mature TFB2M protein with a sequence set forth in SEQ ID NO:12 or SEQ ID NO:16, preferably SEQ ID NO:12;
(ii) a protein that is encoded by a nucleotide sequence set forth in SEQ ID NO:11 or SEQ ID NO:15, preferably SEQ ID NO:11;
(iii) a mature TFB2M protein that is orthologous to the human mature TFB2M protein of (i);
(iv) a protein that is encoded by a nucleotide sequence which is orthologous to the nucleotide sequence described in (ii); and/or
(v) a protein that has at least 60%, 70%, 80% or 90%, preferably at least 90%, 95%, 98% or 99%, e.g. 95%, 96%, 97%, 98%, or 99% sequence identity to the protein of any one of (i) to (iv), preferably (i) or (ii), more preferably (i).

In particular, the mature TFB2M protein may be a human mature TFB2M protein with a sequence set forth in SEQ ID NO:12 or SEQ ID NO:16, preferably SEQ ID NO:12, and/or a protein that has at least 60%, 70%, 80% or 90%, preferably at least 90%, 95%, 98% or 99%, e.g. 95%, 96%, 97%, 98%, or 99% sequence identity to said human mature TFB2M protein. Preferably, the mature TFB2M protein has a sequence set forth in SEQ ID NO:12 or SEQ ID NO:16, preferably SEQ ID NO:12.

As used herein and in the context of the present invention, the immature TFB2M protein may refer to:
(i) a human immature TFB2M protein with a sequence set forth in SEQ ID NO:10, SEQ ID NO:14 or SEQ ID NO:18, preferably SEQ ID NO:10;
(ii) a protein that is encoded by a nucleotide sequence set forth in SEQ ID NO:9, SEQ ID NO:13 or SEQ ID NO:17, preferably SEQ ID NO:9;
(iii) an immature TFB2M protein that is orthologous to the human immature TFB2M protein of (i);
(iv) a protein that is encoded by a nucleotide sequence which is orthologous to the nucleotide sequence described in (ii); and/or
(v) a protein that has at least 60%, 70%, 80% or 90%, preferably at least 90%, 95%, 98% or 99%, e.g. 95%, 96%, 97%, 98%, or 99% sequence identity to the protein of any one of (i) to (iv), preferably (i) or (ii), more preferably (i).

In particular, the immature TFB2M protein may be a human immature TFB2M protein with a sequence set forth in SEQ ID NO:10, SEQ ID NO:14 or SEQ ID NO:18, preferably SEQ ID NO:10, and/or a protein that has at least 60%, 70%, 80% or 90%, preferably at least 90%, 95%, 98% or 99%, e.g. 95%, 96%, 97%, 98%, or 99% sequence identity to said human immature TFB2M protein. Preferably, the immature TFB2M protein has a sequence set forth in SEQ ID NO:10, SEQ ID NO:14, or SEQ ID NO:14, preferably SEQ ID NO:10.

In particular, the immature TFB2M protein, as used herein, contains a N-terminal part that is cleaved off when the mature TFB2M protein is generated.

Accordingly, the mature TFB2M protein, as used herein, does, in particular, not contain, i.e. not at the N-terminal end, a sequence corresponding to the N-terminal part of the immature TFB2M protein that is cleaved off when the mature TFB2M protein is generated.

For example, said N-terminal part of the immature TFB2M protein may have the amino acid sequence set forth in positions 1 to 43 of SEQ ID NO:10 (i.e. as set forth in SEQ ID NO: 42), an amino acid sequence encoded by the nucleic acid sequence set forth in positions 1 to 129 of SEQ ID NO:9 (i.e. as set forth in SEQ ID NO: 41), or an amino acid sequence that has at least 60%, 70%, 80% or 90%, preferably at least 90%, 95%, 98% or 99%, e.g. 95%, 96%, 97%, 98%, or 99% sequence identity to any of said amino acid sequences.

The skilled person has no difficulties in identifying an orthologue of a protein (e.g. mature TFAM or immature TFAM) or a nucleotide encoding such a protein in a different species, e.g. in a different mammal such as, inter alia, a horse, dog, cat, cow, pig, goat, sheep, mouse, rat, guinea pig, rabbit, camel, alpaca, or monkey, for example, by interrogating well known databases such as inter alia Ensembl, Pubmed and/or Genome Browser.

As used herein, the term “sequence identity” is used to describe the sequence relationships between two or more amino acid sequences, proteins (or fragments thereof), or polypeptides (or fragments thereof). The term can be understood in the context of and in conjunction with the terms including: (a) reference sequence, (b) comparison window, (c) sequence identity, (d) percentage of sequence identity, and (e) substantial identity or “homologous”.

A “reference sequence” is a defined sequence used as a basis for sequence comparison. A reference sequence may be a subset of or the entirety of a specified sequence.

A “comparison window” includes reference to a contiguous and specified segment of an amino acid sequence/polypeptide sequence/protein sequence, wherein the amino acid sequence/polypeptide sequence/protein sequence may be compared to a reference sequence. The portion of the amino acid sequence/polypeptide sequence/protein sequence in the comparison window may comprise additions, substitutions, or deletions (i.e., gaps) compared to the reference sequence (which does not comprise additions, substitutions, or deletions) for optimal alignment of the two sequences. For example, the comparison window may be about 20, 50, 100 or 200 amino acid residues in length or longer. Those of skill in the art understand that to avoid a misleadingly high similarity to a reference sequence due to inclusion of gaps in the polynucleotide or polypeptide sequence a gap penalty may be introduced and subtracted from the number of matches.

Methods of alignment of sequences for comparison are well-known in the art. Optimal alignment of sequences for comparison may be conducted by the local homology algorithm of Smith and Waterman, Adv. Appl. Math., 2: 482, 1981; by the homology alignment algorithm of Needleman and Wunsch, J. Mol. Biol., 48: 443, 1970; by the search for similarity method of Pearson and Lipman, Proc. Natl. Acad. Sci. USA, 8: 2444, 1988; by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG), 7 Science Dr., Madison, Wisc., USA; the CLUSTAL program is well described by Higgins and Sharp (1988) Gene 73: 237-244; Corpet et al. (1988) Nucleic Acids Research 16:881-90; Huang, et al. (1992) Computer Applications in the Biosciences, 8:1-6; and Pearson, et al. (1994) Methods in Molecular Biology, 24:7-331. The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York, 1995. New versions of the above programs or new programs altogether will undoubtedly become available in the future, and can be used with the present invention.

Unless otherwise stated, sequence identity/similarity values provided herein refer preferably to the value obtained using the BLAST 2.0 suite of programs, or their successors, using default parameters. Altschul et al. (1997) Nucleic Acids Res, 2:3389-3402. It is to be understood that default settings of these parameters can be readily changed as needed in the future. Evidently, for comparison of amino acid sequences/protein sequences/polypeptide sequences, an algorithm/program directed to the alignment of amino acid sequences/protein sequences/polypeptide sequences should be used, e.g. BLASTP. As those ordinary skilled in the art will understand, BLAST searches assume that proteins or nucleic acids can be modeled as random sequences. However, many real proteins and nucleic acids comprise regions of nonrandom sequences which may be homopolymeric tracts, short-period repeats, or regions enriched in one or more amino acids or nucleic acids. Such low-complexity regions may be aligned between unrelated proteins even though other regions of the protein or nucleic acid are entirely dissimilar. A number of low-complexity filter programs can be employed to reduce such low-complexity alignments. For example, the SEG (Wooten et al. (1993) Comput. Chem. 17:149-163) and XNU (Claverie et al. (1993) Comput. Chem. 17:191-1) low-complexity filters can be employed alone or in combination.

“Sequence identity” in the context of two polypeptide/protein sequences includes reference to the residues in the two sequences which are the same when aligned for maximum correspondence over a specified comparison window, and can take into consideration additions, deletions and substitutions. When percentage of sequence identity is used in reference to proteins it is recognized that residue positions which are not identical often differ by conservative amino acid substitutions, where amino acid residues are substituted for other amino acid residues with similar chemical properties (for example, charge or hydrophobicity) and therefore do not deleteriously change the functional properties of the molecule. Where sequences differ in conservative substitutions, the percent sequence identity may be adjusted upwards to correct for the conservative nature of the substitution. Sequences which differ by such conservative substitutions may be said to have sequence similarity. Approaches for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, for example, according to the algorithm of Meyers and Miller, Computer Applic. Biol. Sci., 4: 11-17, 1988, for example, as implemented in the program PC/GENE (Intelligenetics, Mountain View, Calif., USA).

“Percentage of sequence identity” refers, in particular, to the value determined by comparing two optimally aligned sequences over a comparison window, wherein the portion of the amino acid/peptide/protein sequence in the comparison window may comprise additions, substitutions, or deletions (i.e., gaps) as compared to the reference sequence (which does not comprise additions, substitutions, or deletions) for optimal alignment of the two sequences. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity.

Furthermore, the mature and/or active TFAM protein may be contained within the mitochondrial core transcription initiation complex. Thus, the interaction of TFAM protein with TFB2M protein may occur, in particular, within the mitochondrial core transcription initiation complex.

Furthermore, the mature and/or active TFAM protein may promote the function of mitochondria, in particular (i) the transcription of mitochondrial genes, (ii) the replication of mitochondrial DNA, and/or (iii) the production of ATP, e.g. as illustrated in the appended Examples.

As described above, in the context of the invention, the level of mature and/or active TFAM protein may correspond to
(i) the amount and/or concentration of mature TFAM protein, preferably the amount of mature TFAM protein,
(ii) the number of interactions of TFAM protein with at least one other mitochondrial protein (e.g. TFB2M) and/or mitochondrial DNA to which the mature TFAM protein binds specifically, and/or
(iii) the ratio of the amount of mature TFAM protein over the amount of immature TFAM protein.

In particular, the number of interactions of TFAM protein with at least one other mitochondrial protein (e.g. TFB2M), and/or mitochondrial DNA to which the mature TFAM protein binds specifically, is indicative of the amount of active TFAM protein.

The amount and/or concentration of mature TFAM protein (e.g. in mitochondria) may be determined, for example, by employing at least one inventive binding molecule and/or kit provided herein, and/or by methods illustrated in the appended Examples. The same applies for determining the amount and/or concentration of immature TFAM protein (e.g. in the cytonucleoplasm).

A protein to which TFAM protein binds specifically in the mitochondria may be considered a binding partner of mature and/or active TFAM protein. For example, the binding may be considered specific, when the dissociation constant (K_{d}) is 10- or smaller, preferably 104 or smaller, e.g. as determined by an electromobility shift assay (EMSA) or surface plasmon resonance (e.g. Biacore).

Furthermore, the mature and/or active TFAM protein may specifically bind to mitochondrial DNA in general (i.e. it may be localized and/or enriched at the mitochondrial DNA), and/or it may specifically to a certain mitochondrial DNA sequence. Binding of TFAM protein to mitochondrial DNA in general may be measured by methods known in the art, e.g. by chromatin immunoprecipitation methods.

The binding to a certain mitochondrial DNA sequence may be considered specific, when TFAM binds preferably (e.g. above background noise) to this sequence, e.g. as determined by ChIP-seq and/or EMSA. This type of binding is typically referred to as “sequence specific binding”. For example, a specific mitochondrial DNA binding sequence of TFAM and/or TFB2M may be a TFAM and TFB2M consensus binding sequence, e.g., inter alia, AAAGATAAAATTTGAAAT or AAAGACACCCCCCACAG, or a sequence that has at least 60%, 70%, 80%, 90% or 95% sequence identity to such an exemplary consensus sequence. In particular, the binding of TFAM protein to a protein binding partner, mitochondrial DNA in general (i.e. non-sequence specific binding), and/or a certain mitochondrial DNA sequence (i.e. sequence-specific binding) has a biological effect, e.g. it may promote the mitochondrial biogenesis as described herein. Thus, the number and/or amount of interactions of TFAM protein with at least one other mitochondrial protein and/or mitochondrial DNA to which the mature TFAM protein binds specifically (non-sequence specific and/or sequence-specific) is indicative of the level (e.g amount) of mature and/or active TFAM protein and/or the activity level of TFAM protein.

Suitable protein binding partners of TFAM include mitochondrial RNA polymerase, nuclear respiratory factor 1, peroxisome proliferator-activated receptor gamma coactivator 1-alpha, GA binding protein transcription factor subunit alpha, cytochrome C, mitochondrial transcription factor B1, and/or single-stranded DNA-binding protein 1. In the context of the invention, the binding partner of TFAM protein, i.e. mature TFAM protein, is preferably TFB2M protein, i.e. mature TFB2M protein.

Furthermore, the amount of mature and/or active TFAM protein may correspond to the amount of mature TFAM protein normalized by the amount of TNF receptor associated protein 1 (TRAP1), in particular the amount of TRAP1 protein, i.e. in the mitochondria.

Furthermore, the amount of immature TFAM protein may correspond to the amount of immature TFAM protein normalized by the amount of β-actin, in particular the amount of β-actin protein, i.e. in the cytonucleoplasm.

The term “cytonucleoplasm”, as used herein refers to the entire content of a cell without mitochondria, in particular to the cytoplasm (except for mitochondria) including, inter alia, the cytosol, endoplasmatic reticulum, golgi, lysosome, and the nucleus. Preferably, neither the cytonucleoplasm nor the mitochondria include the cell plasma membrane. The mitochondria may be separated or distinguished from the cytonucleoplasm in vitro and/or in silico, e.g. as illustrated in the appended Examples and/or by methods known in the art.

Furthermore, the level of mature and/or active TFAM protein may correspond to the ratio (R) of the amount of mature and/or active TFAM protein (M) normalized by the amount of TRAP1 protein (T) over the amount of immature TFAM protein (I) normalized by the amount of β-actin (B), as described by the formula: R=(M/T)/(I/B).

In particular, the amount of mature and/or active TFAM protein and the amount of TRAP1 protein may be determined in the mitochondria, whereas the amount of immature TFAM protein and β-actin protein may be determined in the cytonucleoplasm.

Preferably herein, as further described above, the level of mature and/or active TFAM protein may correspond to the number of interactions of TFAM protein with mitochondrial transcription factor B2 (TFB2M) protein.

In the context of the inventive methods provided herein, determining the level of mature and/or active TFAM protein may comprise quantifying the interaction of TFAM protein with at least one other mitochondrial protein and/or mitochondrial DNA to which the mature TFAM protein binds specifically.

Quantifying the interaction of TFAM protein with mitochondrial DNA may be done, e.g. by chromatin immunopreciptiation (ChIP) using an anti-TFAM antibody, for example by ChIP-seq.

It is also possible to infer the level of mature and/or active TFAM protein by a functional assay, e.g. a reporter (e.g. luciferase) assay, wherein the activation of a TFAM-specific promoter and/or enhancer by mature and/or active TFAM protein leads to expression of a reporter protein (e.g. a luciferase or a fluorescent protein).

Preferably, however, determining the level of mature and/or active TFAM protein comprises quantifying the interaction of TFAM protein with at least one other mitochondrial protein to which the mature TFAM protein binds specifically.

In some embodiments of the invention, the amount and/or number of interactions of TFAM with a protein binding partner may be quantified by an electromobility shift assay (EMSA), Co-Immunopreciptiation (CoIP) and/or a FRET/FLIM assay.

Preferably, however, in the context of the present invention, quantifying the interaction of TFAM protein with at least one other mitochondrial protein to which the mature and/or active TFAM protein binds specifically, preferably TFB2M protein, e.g. mature TFB2M protein, comprises the steps of
(a) contacting the sample with a pair of binding molecules,
   wherein one of said binding molecules specifically binds TFAM protein, and
   wherein the other of said binding molecules specifically binds said other mitochondrial protein to which the mature TFAM protein binds specifically (e.g. TFB2M protein), and
(b) generating a detectable signal when said two binding molecules are in close proximity to each other, in particular when the distance between said two binding molecules is about 100, 80, 60 or 40 nm or less, preferably about 40 nm or less, e.g. about 40 nm or about 20 nm,
   thereby quantifying said interaction.

Hence, determining the level of mature and/or active TFAM protein may preferably comprise quantifying the interaction of TFAM protein with TFB2M protein, i.e. the number of interactions of TFAM protein with TFB2M protein in the sample.

Evidently, the number of interactions in a sample does not necessarily refer to all interactions physically present in the sample, but may refer to a representative subset thereof, i.e. to the interactions detected by a measurement method described herein.

In the context of the invention, e.g. in the context of quantifying the interaction of TFAM protein with at least one other mitochondrial protein to which the mature TFAM protein binds specifically, a binding molecule, i.e. a protein binding molecule, may be an antibody, a monobody, a nanobody, or an aptamer. In principle, however, any molecule may be considered a binding molecule if it specifically binds to its target (e.g. mature TFAM protein), preferably with a K_{D} of 10⁻⁸ M or smaller, and preferably does not bind to other molecules in the sample (e.g. immature TFAM protein) with such a low K_{D}. Thus, a binding molecule may also be called an “affinity binder”. Preferably, the binding molecule, in context of the invention, is an antibody.

An antibody, as used herein, may be a polyclonal antibody, a monoclonal antibody, a full antibody (immunoglobulin), a F(ab)-fragment, a F(ab)₂-fragment, a single-chain antibody, a chimeric antibody, a CDR-grafted antibody, a bivalent antibody-construct, a bispecific single chain antibody, a synthetic antibody or a cross-cloned antibody and the like.

A monobody, as used herein, refers, in particular, to a synthetic binding protein that is constructed using a fibronectin type III domain (FN3) as a molecular scaffold.

A nanobody, as used herein, refers, in particular, to a single-domain antibody (sdAb), which is an antibody fragment consisting of a single monomeric variable antibody domain. Thus, the term “antibody” may further encompass nanobodies.

An aptamer, as used herein, refers, in particular, to an oligonucleotide or peptide molecule that binds to a specific target molecule. For example, aptamers include (i) DNA or RNA or XNA aptamers which may consist of, usually short, strands of oligonucleotides, and (ii) peptide aptamers which consist of one or more short variable peptide domains, and which may be attached at both ends to a protein scaffold.

Furthermore, in the context of the present invention, generating a detectable signal, may comprise the steps of
(a) generating an oligonucleotide template when said two binding molecules (e.g. affinity binders) are in close proximity to each other, e.g. about 40 nm or less apart from each other, and
(b) amplifying and/or extending said oligonucleotide template, in particular wherein the amplified and/or extended oligonucleotide is labeled, preferably with a fluorophore.

For example, the oligonucleotide template may be formed by ligation of at least two oligonucleotides (e.g. proximity oligonucleotides) and may be linear or circularized (e.g. as in PLA), and/or the oligonucleotide template may be formed by a change in secondary or tertiary structure of an oligonucleotide (e.g. proximity oligonucleotide) and/or by binding of at least one other oligonucleotide to said oligonucleotide (e.g. as in ProxHCR).

Preferably, the amplified and/or extended oligonucleotide may be generated and detected at the position where said two binding molecules are located.

A suitable fluorophore may be, inter alia, Alexa Fluor 488, Alexa Fluor 546, ATTO 390, ATTO 488, ATTO 565, ATTO 680, ATTO 700, Cy 3, Cy 3.5, Cy 5, Cy 5.5, Cy 7, FITC, TRITC, Texas Red, FAM, TAMRA etc.

The detectable signal may be quantified by imaging, flow cytometry and/or a point-of-care device, e.g. as illustrated in the appended Examples.

Furthermore, each of the two binding molecules (e.g. affinity binders) may comprise an oligonucleotide (e.g. a proximity oligonucleotide) and/or is specifically bound by a secondary binding molecule comprising such an oligonucleotide. In particular, the two oligonucleotides linked to the two binding molecules can form an oligonucleotide template when said two binding molecules are in close proximity to each other, as described herein. Furthermore, said oligonucleotide template can be amplified and/or extended. For example, an oligonucleotide (e.g. a proximity oligonucleotide) comprised in (e.g. attached/conjugated to) a binding molecule may have a length of about 10 bp to about 200 bp, preferably about 20 bp to about 100 bp.

In preferred embodiments, the two binding molecules refer to an antibody specifically binding TFAM protein and an antibody specifically binding TFB2M protein.

Preferably, a binding molecule (i.e. a protein binding molecule) is an antibody conjugated to an oligonucleotide (e.g. proximity oligonucleotide) and/or a binding molecule is specifically bound by an antibody conjugated to an oligonucleotide (e.g. proximity oligonucleotide).

In particularly preferred embodiments, quantifying the interaction of TFAM protein with at least one other mitochondrial protein to which the mature TFAM protein binds specifically (e.g. TFB2M) comprises performing a proximity ligation assay (PLA) and/or a proximity-dependent initiation of hybridization chain reaction (proxHCR), e.g. as described in the appended Examples. ProxHCR is further described in Koos (2014), Nature Communications 6, and the published patent application US20170009278. PLA is further described in Söderberg (2006), Nat Methods, 3(12) and Clausson (2015), Sci Rep 5, 12317.

Preferably herein, e.g. in the context of the inventive diagnostic and/or prognostic methods, the sample is a blood sample. Further suitable samples may be from muscle, brain, skin, heart, liver, kidney, and/or lungs.

Furthermore, the sample may comprise a cell, a cell or tissue lysate, and/or a cell or tissue extract, e.g. it may be a whole blood lysate. In particular, the cell comprises mitochondria. Preferably, the cell is a peripheral blood mononuclear cell. Preferably, the tissue is blood. In particular, for determining the level of mature and/or active TFAM protein, the sample may comprise mitochondria. Thus, the cell extract preferably comprises mitochondria, and preferably does not comprise the cell nucleus and cytoplasm.

Thus, in some embodiments, the level of mature and/or active TFAM protein in the mitochondria is determined (and not in the entire cell or cytonucleoplasm). This may be done, for example, directly by isolating mitochondria in vitro and/or in silico (e.g. in a microscopic image of the cell), or indirectly by measuring the interaction of TFAM protein with TFB2M protein (which only occurs in the mitochondria). For isolating mitochondria, e.g. in silico, mitochondrial markers may be employed which allow distinguishing mitochondria from the rest of the cell. Suitable mitochondrial markers may be TRAP1, VDAC and/or a MitoTracker™ (e.g., inter alia, Benzoxazolium, 2-[3-[5,6-dichloro-1,3-bis[[4-(chloromethyl)phenyl]methyl]-1,3-dihydro-2H-benzimidazol-2-ylidene]-1-propenyl]-3-methyl-, chloride 201860-17-5 or 1H,5H,11H,15H-Xantheno[2,3,4-ij:5,6,7-i′j′]diquinolizin-18-ium, 9-[4-(chloromethyl)phenyl]-2,3,6,7,12,13,16,17-octahydro-, chloride 167095-09-2).

In the context of the present invention, the diagnosis or prognosis may be made regardless of the level (i.e. amount) of TFAM mRNA in the sample, and/or the level (i.e. amount) of total, immature and/or cytonucleoplasmic TFAM protein in the sample. In particular, the amount of total TFAM protein, as used herein, refers to the combined amount of mature and immature TFAM protein, for example, in the cell, mitochondria or cytonucleoplasm, as indicated.

Moreover, the diagnosis or prognosis may be confirmed, when the level of TFAM mRNA in said sample and/or the level of total, immature and/or cytonucleoplasmic TFAM protein in said sample is unaltered or altered in the opposite direction as the level of mature and/or active TFAM protein. As explained above and illustrated in the appended Examples, the immature TFAM protein cannot be used as a biomarker for prognosing the outcome of an organ dysfunction. For example, when the level (e.g. amount) of mature and/or active TFAM protein in the sample is below the reference level and the level (e.g. amount) of total or immature TFAM protein in the sample is unchanged or elevated, still a negative outcome may be prognosed. As a further example, when the level (e.g. amount) of mature and/or active TFAM protein in the sample is equal to or higher than the reference level and the level (e.g. amount) of total or immature TFAM protein in the sample is unchanged or reduced, still a positive outcome may be prognosed.

Furthermore, the invention relates to a method of treating a patient in need suffering from an organ dysfunction, wherein said patient has an organ dysfunction with a negative outcome and/or a very severe organ dysfunction, and wherein said method comprises administering to said patient in need of medical intervention and/or treatment a therapeutically effective amount of a supportive drug, wherein said supportive drug is selected from at least one drug from the group consisting of: (i) an antioxidant, (ii) human immunoglobulins, (iii) a chemotherapeutic agent, and (iii) a hydrocortisone.

Preferably, the outcome of the organ dysfunction is prognosed to be negative by the inventive in vitro method for prognosing the outcome of an organ dysfunction in a patient provided herein, and/or the organ dysfunction is diagnosed to be very severe (high degree of severity of the organ dysfunction) by the inventive in vitro method for diagnosing the degree of severity of an organ dysfunction in a patient provided herein.

Of note, the diagnosis and/or prognosis do not have to be necessarily made by the same person and/or institution where the patient is treated.

Preferably herein, e.g. in the context of the inventive treatment methods, said supportive drug is an antioxidant.

Said antioxidant may be, for example, ascorbic acid, mitoquinone mesylate and/or n-acetylcysteine. Preferably, said antioxidant is administered intravenously.

Furthermore, said human immunoglobulins may be, for example, intravenously administered IgA- and/or IgM-enriched human immunoglobulins.

Furthermore, said chemotherapeutic agent, may be, inter alia, comprise cyclosporine and/or epirubicin.

Furthermore, the invention relates to a supportive drug for use in treating a patient, wherein said patient suffers from an organ dysfunction with a negative outcome and/or a very severe dysfunction. As regards the patient and the supportive drug, the same applies as is described herein in the context of the inventive method of treating a patient in need suffering from an organ dysfunction, wherein said patient has an organ dysfunction with a negative outcome and/or a very severe organ dysfunction provided herein.

A supportive drug, as used herein, may be considered an experimental drug, as described herein. In particular, a supportive and/or experimental drug may be a drug which is not necessarily beneficial for treating patients with an organ dysfunction such as septic patients. This may be the case, for example, when a beneficial effect is not well established, the therapeutic efficacy varies substantially between individual patients, and/or because the experimental drug has undesired side effects that are preferably avoided. For example, antioxidants are often administered to septic patients without a real benefit (Fujii (2020), JAMA; 323(5)). However, it is contemplated that only patients suffering from an organ dysfunction with a negative outcome and/or a very severe organ dysfunction may benefit from an antioxidant, i.e. an intravenously administered antioxidant. In other words, a supportive and/or experimental drug is ideally only administered to a patient suffering from an organ dysfunction, when it is really necessary and/or when it is likely that the patient benefits from such a supportive drug. Thus, when the outcome of an organ dysfunction is prognosed to be positive and/or when it is diagnosed that the organ dysfunction is not very severe, preferably no supportive and/or experimental drug is to be administered to the patient. In contrast, when the outcome of an organ dysfunction is prognosed to be negative and/or when it is diagnosed that the organ dysfunction is very severe, it may be advisable to administer a supportive and/or experimental drug to the patient. It is plausible that the overall outcome of the organ dysfunctions changes to the positive, when only the patients are treated with a supportive and/or experimental drug which suffer from a very severe organ dysfunction.

Thus, the invention further relates to a method of treating a patient in need suffering from an organ dysfunction, wherein said patient has an organ dysfunction with a positive outcome and/or an organ dysfunction with a low degree of severity, and wherein said method comprises treating said patient in an intensive care unit without administering to said patient in need of medical intervention and/or treatment a supportive and/or experimental drug as described herein.

Preferably, the outcome is prognosed to be positive by the inventive in vitro method for prognosing the outcome of an organ dysfunction in a patient provided herein, and/or the organ dysfunction is diagnosed to be not very severe (low degree of severity of the organ dysfunction) by the inventive in vitro method for diagnosing the degree of severity of an organ dysfunction in a patient provided herein.

Furthermore, the prognostic or diagnostic method according to the invention may further comprise a step of indicating a method of treatment according to an inventive treatment method provided herein based on the prognosis of the outcome of the organ dysfunction and/or the degree of severity of the organ dysfunction.

Furthermore, the invention relates to an in vitro method for diagnosing the presence of an organ dysfunction in a subject, wherein said method comprises the steps of
(a) determining the level of mature and/or active mitochondrial transcription factor A (TFAM) protein in a sample from said subject;
(b) comparing the level of mature and/or active TFAM protein to a control level; and
(c) determining that an organ dysfunction is present in said subject when the level of mature and/or active TFAM protein is lower than the control level, and/or
   determining that no organ dysfunction is present in said subject when the level of mature and/or active TFAM protein is equal to or higher than the control level.

Preferably, said subject is a patient suffering from an infection and/or inflammation, as described herein. Furthermore, said subject may be suspected of suffering from an organ dysfunction as described herein.

As regards step (a) of determining the level of mature and/or active TFAM protein, the level of mature and/or active TFAM protein, the mature and/or active TFAM protein, and the organ dysfunction, the same applies as is described herein in the context of the inventive in vitro method for prognosing the outcome of an organ dysfunction in a patient and/or the inventive in vitro method for diagnosing the degree of severity of an organ dysfunction in a patient.

The control level is, in particular, indicative of the absence of an organ dysfunction. The control level may be determined by analyzing the level of mature and/or active TFAM protein in samples from
(i) a plurality of negative control subjects that are not suffering or recovering from an organ dysfunction, infection and/or inflammation as described herein, and/or
(ii) a plurality of positive control patients diagnosed with an organ dysfunction as described herein.

Preferably, the level of mature and/or active TFAM protein in the samples from said negative control subjects and/or positive control patients is determined by the same measurement method that is employed in step (a) of the inventive method for diagnosing the presence of an organ dysfunction provided herein.

Furthermore, the negative and positive control subjects/patients may be considered a reference group, just like the reference patients described herein in the context of the reference level. However, said negative and positive control subjects/patients are a different group of subjects than said reference patients. The control level refers, in particular, to a threshold which allows to separate the negative control subjects (which do not have an organ dysfunction) from the positive control patients (which do have an organ dysfunction) in a useful and/or optimal way. In one embodiment, the negative control subjects are suffering from an infection and/or inflammation but are not suffering or recovering from an organ dysfunction.

Thus, the “control” level, as used herein, is normally a different level than the “reference” level, as used herein. For example, when the level of mature and/or active TFAM protein in a sample from a subject to be diagnosed is lower than the control level, it is diagnosed that said subject suffers from an organ dysfunction. However, based on such an assessment, it cannot be diagnosed or prognosed whether the outcome of the organ dysfunction is negative or positive, (and/or very severe or not very severe). For such an assessment, the reference level may be applied, as described herein in the context of the inventive methods for prognosing the outcome of an organ dysfunction and/or diagnosing the degree of severity of an organ dysfunction, as provided herein.

In particular, the control level may be selected such that
(i) the level of mature and/or active TFAM protein in the samples of at least about 60%, 70%, 80%, 90%, preferably at least about 95%, 97% or 99 of the negative control subjects is equal to or higher than said control level, and/or
(ii) the level of mature and/or active TFAM protein in the samples of at least about 60%, 70%, 80%, 90%, or at least about 95%, 97% or 99%, e.g. at least about 80%, of the positive control patients is lower than said control level.

Furthermore, it has been surprisingly found by the inventors, as already indicated above, that the level of mature and/or active TFAM protein in the samples of septic patients was about 70% lower than in samples from healthy negative control subjects that were not suffering from sepsis. This means that the samples from the septic patients had about 30% of the level of mature and/or active TFAM protein of the samples from the negative control subjects. Or in other words, the level of mature and/or active TFAM protein was decreased by about 70% to a level of about 30% in septic patients compared to healthy negative control subjects.

As demonstrated in the appended Examples, this finding was confirmed by two different measurement methods. In particular, as shown in FIG. 7g, the amount of mature mitochondrial TFAM protein normalized to the amount of TRAP1 protein measured by western blot was 72% lower in samples from septic patients than in corresponding samples from healthy negative control subjects. Furthermore, as shown in FIG. 9a, the number of interactions of TFAM protein with TFB2M protein measured by a proximity ligation assay (PLA) was 74% lower in samples from septic patients than in corresponding samples from healthy negative control subjects.

Thus, the invention further relates to a method of detecting an abnormal level of mature and/or active mitochondrial transcription factor A (TFAM) protein in a sample from a patient, wherein said method comprises
a. measuring the level of mature and/or active TFAM protein in the sample,
b. determining whether the sample is abnormal, wherein the sample is determined to be abnormal if the level of mature and/or active TFAM protein is at least about 20% lower than the amount determined for a reference sample.

In particular, said reference sample is derived from at least one negative control subject (e.g. at least one healthy subject) that is not suffering or recovering from an organ dysfunction, infection and/or inflammation as described herein.

Evidently, a level of mature and/or active TFAM protein that is at least about 20% lower than the amount determined for a reference sample, further means that said level is 80% or less of the amount in said reference sample.

Preferably herein, i.e. in context of the method of detecting an abnormal level of mature and/or active TFAM protein in a sample from a patient, the term about means +/−20%, preferably +/−10%, of the given value.

Preferably, the sample from said patient is determined to be abnormal if the level of mature and/or active TFAM protein is at least about 30%, about 40%, about 50%, about 60%, about 70%, or about 80%, preferably at least about 50%, more preferably at least about 70%, e.g. about 70%, lower than the amount determined for said reference sample.

Preferably, the inventive method of detecting an abnormal level of mature and/or active TFAM protein in a sample from a patient further comprises:
c. reporting to said patient whether said sample is determined to be abnormal or normal.

In particular, an abnormal level of mature and/or active TFAM protein in a sample or an abnormal sample, is indicative of the presence of an organ dysfunction, e.g. sepsis, as described herein.

Thus, it may be further reported that a patient that is reported to have an abnormal level of mature and/or active TFAM protein suffers from an organ dysfunction as described herein.

Furthermore, it has been surprisingly found by the inventors that the level of mature and/or active TFAM protein in the samples of septic patients that died within one month was about 60% lower than in samples from septic patients that survived for at least one month. In other words, the level of mature and/or active TFAM protein was decreased by about 60% to a level of about 40% in patients in which the organ dysfunction had a negative outcome compared to patients in which the organ dysfunction had a positive outcome.

This finding was confirmed by two different measurement methods, as demonstrated in the appended Examples. In particular, as shown in FIG. 10b, the amount of mature mitochondrial TFAM protein normalized to the amount of TRAP1 protein measured by western blot was 70% lower in samples from septic patients that died within one month than in corresponding samples from septic patients that survived for at least one month. Furthermore, as shown in FIG. 12, the number of interactions of TFAM protein with TFB2M protein measured by a proximity ligation assay (PLA) was 63% lower in samples from septic patients that died within one month than in corresponding samples from septic patients that survived for at least one month.

Furthermore, it was found that the level of mature and/or active TFAM protein in the samples of septic patients that died within one month was about 90% lower than in samples from healthy negative control subjects. In particular, it was determined by PLA, as demonstrated in FIGS. 9a and 12, that the number of interactions of TFAM protein with TFB2M protein in samples of septic patients that died within one month was 87% lower than in samples from healthy negative control subjects, wherein the number of interactions of TFAM protein with TFB2M protein in samples of septic patients that survived for at least one month was 66% lower than in samples from healthy negative control subjects.

Thus, in the context of the inventive method of detecting an abnormal level of mature and/or active TFAM protein in a sample from a patient, said patient having reported an abnormal level of mature and/or active TFAM protein may be also reported to expect death within one month, in particular when said level of mature and/or active TFAM protein in a sample from said patient is at least about 40%, about 50%, about 60%, about 70%, or about 80%, preferably at least about 80%, more preferably at least about 90% lower than the amount determined for said reference sample.

Furthermore, it may be reported that the patient suffers from a very severe organ dysfunction as described herein, when the level of mature and/or active TFAM protein is at least about 40%, about 50%, about 60%, about 70%, or about 80%, preferably at least about 80%, more preferably at least about 90% lower in a sample from said patient than the amount determined for said reference sample.

In contrast, in the context of the inventive method of detecting an abnormal level of mature and/or active TFAM protein in a sample from a patient, said patient having reported a normal level of mature and/or active TFAM protein may be also reported to expect survival for at least one month and/or ICU-freedom within one week, i.e. because said patient is likely not suffering from an organ dysfunction.

However, as illustrated in the appended Examples, a patient with a moderately abnormal level of mature and/or active TFAM protein may still expect survival for at least one month and/or ICU-freedom within one week, e.g. when the level of mature and/or active TFAM protein in a sample from said patient is less than 40%, e.g about 20% to about 30%, lower than the amount determined for a reference sample from at least one healthy negative control subject. A patient reported to have a moderately abnormal level of mature and/or active TFAM protein (that is e.g. about 20% to about 30% lower than in the reference sample) may be further reported to have an organ dysfunction that is not very severe.

In particular, in the context of the inventive method of detecting an abnormal level of mature and/or active TFAM protein in a sample from a patient, said patient is suspected of having an organ dysfunction. Preferably, the organ dysfunction is sepsis.

As regards step (a) of measuring the level of mature and/or active TFAM protein in the sample, the level of mature and/or active TFAM protein, the mature and/or active TFAM protein, and the organ dysfunction, the same applies as is described herein in the context of the inventive in vitro method for prognosing the outcome of an organ dysfunction in a patient and/or the inventive in vitro method for diagnosing the degree of severity of an organ dysfunction in a patient, e.g. in context of determining the level of mature and/or active TFAM protein.

Thus, the invention further relates to a method of treating organ dysfunction in a patient, wherein said method comprises administering to the patient an antibiotics, and/or intravenously a crystalloid or saline solution, wherein said patient was reported as having an abnormal level of mature and/or active TFAM protein, wherein said level of mature and/or active TFAM protein in a sample obtained from said patient was determined to be at least about 20%, for example, at least about 30%, about 40%, about 50%, about 60%, about 70%, or about 80%, preferably at least about 50%, more preferably at least about 70%, e.g. about 70%, lower than the amount determined for a reference sample, as described herein. Preferably said antibiotics, and/or crystalloid or saline solution is administered within 1, 2 or 3 hours, preferably within one hour, after the patient was reported as having an abnormal level of mature and/or active TFAM protein, as described herein.

Thus, the invention further relates to a method of treating a patient in need suffering from an infection and/or inflammation, wherein said patient in need has been diagnosed to have an organ dysfunction by the inventive in vitro method for diagnosing the presence of an organ dysfunction in a subject provided herein, and wherein said method of treating comprises treating said patient in an intensive care unit, administering to said patient in need an antibiotics, and/or intravenously a crystalloid or saline solution. Preferably said treatment is initiated within 1, 2 or 3 hours, preferably within one hour, after the diagnosis has been made.

In some embodiments, the inventive in vitro method for diagnosing the presence of an organ dysfunction in a subject provided herein may further comprises prognosing the outcome of the organ dysfunction according to the inventive in vitro method for prognosing the outcome of an organ dysfunction provided herein, and/or diagnosing the severity of the organ dysfunction according to the in vitro method for diagnosing the degree of severity of an organ dysfunction.

Thus, in some embodiments, the inventive method of treating a patient in need suffering from an infection and/or inflammation provided herein comprises
I) administering to said patient in need of medical intervention and/or treatment a therapeutically effective amount of a supportive drug, wherein said supportive drug is selected from at least one drug from the group consisting of: (i) an antioxidant, (ii) human immunoglobulins, (iii) a chemotherapeutic agent, and (iii) a hydrocortisone, when said patient has an organ dysfunction with a negative outcome and/or a very severe organ dysfunction, or
II) not administering to the patient such a supportive drug, when said patient has an organ dysfunction with a positive outcome and/or an organ dysfunction with a low degree of severity.

Thus, the invention further relates to a method of treating organ dysfunction in a patient, wherein said method comprises administering to the patient a supportive drug, wherein said supportive drug is selected from at least one drug from the group consisting of: (i) an antioxidant, (ii) human immunoglobulins, (iii) a chemotherapeutic agent, and (iii) a hydrocortisone, as described herein, wherein said patient was reported as having an abnormal level of mature and/or active TFAM protein, wherein said level of mature and/or active TFAM protein in a sample obtained from said patient was determined to be at least about 40%, for example, at least about 50%, about 60%, about 70%, about 80%, or about 90%, preferably at least about 80%, more preferably at least about 90%, e.g. about 90%, lower than the amount determined for a reference sample, as described herein.

The invention also relates to a method of treating an organ dysfunction in a patient, wherein said method comprises administering to the patient a supportive drug, wherein said supportive drug is selected from at least one drug from the group consisting of: (i) an antioxidant, (ii) human immunoglobulins, (iii) a chemotherapeutic agent, and (iii) a hydrocortisone, wherein said patient was reported as having a decreasing level of mature and/or active TFAM protein, and wherein said level of mature and/or active TFAM protein in samples repeatedly obtained from said patient has decreased by at least about 10% within one day, preferably by at least 10% per day, e.g. for two or three consecutive days.

Furthermore, such a supportive drug may be withdrawn or omitted, when the level of mature and/or active TFAM protein in samples repeatedly obtained from the patient has increased by at least about 10% within one day, preferably by at least 10% per day, e.g. for two or three consecutive days.

Furthermore, the invention relates to a binding molecule, preferably an antibody, specifically binding the mature and/or active TFAM protein. Preferably, said binding molecule does not specifically bind immature TFAM protein. Such a binding molecule further refers to “binding molecule MAT” herein.

Furthermore, the invention relates to a binding molecule, preferably an antibody, specifically binding the immature TFAM protein. Preferably said binding molecule does not specifically bind mature and/or active TFAM protein. Such a binding molecule further refers to “binding molecule IMM” herein.

The inventive antibody provided herein may be a polyclonal antibody, a monoclonal antibody, a full antibody (immunoglobulin), a F(ab)-fragment, a F(ab)₂-fragment, a single-chain antibody, a chimeric antibody, a CDR-grafted antibody, a bivalent antibody-construct, a bispecific single chain antibody, a synthetic antibody or a cross-cloned antibody or the like.

Polyclonal or monoclonal antibodies or other antibodies (derived therefrom) can be routinely prepared using, inter alia, standard immunization protocols; see Ed Harlow, David Lane, (December 1988), Antibodies: A Laboratory Manual, Cold Spring Harbor Laboratory; or Ed Harlow, David Lane, (December 1998), Portable Protocols (Using Antibodies): A Laboratory Manual 2^{nd} edition, Cold Spring Harbor Laboratory.

For example, for generating an antibody specifically binding the mature and/or active TFAM protein, immunization may involve the intraperitoneal or subcutaneous administration of the mature TFAM protein (and/or fragments thereof) as defined herein to a mammal (e.g. rodents such as mice, rats, hamsters and the like). Preferably, a full-length mature TFAM protein is used, preferably wherein said full-length mature TFAM protein is in its native folding state, e.g. it is isolated from mitochondria without altering the folding state.

Furthermore, for example, for generating an antibody specifically binding the immature TFAM protein, immunization may involve the intraperitoneal or subcutaneous administration of the immature TFAM protein (and/or fragments thereof) as defined herein to a mammal (e.g. rodents such as mice, rats, hamsters and the like). Preferably, fragments of the immature TFAM protein are used, wherein the fragment preferably bears the N-terminal part (or a fragment thereof) as defined herein.

A preferred fragment of the above mentioned immature TFAM protein may consist of from 15 to 25 contiguous amino acids. Accordingly, a fragment of the immature TFAM protein may consist of 15, 16, 17, 18, 19, 20, 21, 22, 23, 24, or 25 contiguous amino acids. In particular, a fragment of the above mentioned immature TFAM protein may preferably consist of from 15 to 25 contiguous amino acids within positions 1 to 50, preferably within positions 1 to 42, of the amino acid sequence shown in SEQ ID NO: 2. Thus, a fragment of the above mentioned immature TFAM protein may preferably consists of 15, 16, 17, 18, 19, 20, 21, 22, 23, 24, or 25 contiguous amino acids within positions 1 to 50, preferably within positions 1 to 42, of the amino acid sequence shown in SEQ ID NO: 2.

Positions 1 to 42 of SEQ ID NO:2 are further shown in SEQ ID NO:40.

Methods for the preparation and screening of antibodies that specifically bind to or specifically recognize either (i) the mature TFAM protein and preferably not the immature TFAM protein, or (ii) the immature TFAM protein and preferably not the mature TFAM protein, are known in the art. For example, antibodies recognizing either the mature TFAM protein or immature TFAM protein may be affinity purified. ELISA is commonly used for screening sera and/or assaying affinity column fractions. Western Blots can be used to demonstrate that the antibody can detect the actual protein of interest and to evaluate whether the antibody only recognizes the protein of interest, or if it cross-reacts with other proteins. For example, it is further illustrated in the appended Examples that Western Blots allow to distinguish the mature TFAM protein (˜24 kDa) from the immature TFAM protein (˜29 kDa). Furthermore, reference antibodies which specifically recognize the total TFAM protein but which cannot distinguish the mature TFAM protein from the immature TFAM protein are readily available, e.g. as shown in the respective Western Blots in the appended Examples.

A person skilled in the art is in the position to apply and to adapt the teaching of these documents for the generation and validation of antibodies specifically binding to or specifically recognizing either the mature TFAM protein or the immature TFAM protein as defined herein in context of the present invention.

For example, in the context of the inventive binding molecule provided herein, binding may be considered specific, when the dissociation constant (K_{D}) is 10⁻⁸ M or smaller, e.g. as determined by surface plasmon resonance (e.g. Biacore) and/or an electromobility shift assay (EMSA).

In the context of the invention, e.g. the diagnostic and/or prognostic methods, determining the level of mature and/or active TFAM protein may comprise contacting the sample with
(i) the binding molecule MAT, or
(ii) the binding molecule IMM and another binding molecule (TOT), preferably an antibody, wherein said binding molecule TOT specifically binds mature and/or active TFAM protein and immature TFAM protein (i.e. the total TFAM protein), and
   in case of (ii) inferring the level of mature and/or active TFAM by comparing the signals of the binding molecule IMM and the other binding molecule TOT.

Furthermore, the invention relates to a kit comprising a primary binding molecule (e.g. a primary antibody) specifically binding TFAM protein (anti-TFAM), and/or a primary binding molecule (e.g. a primary antibody) specifically binding TFB2M (anti-TFB2M). Anti-TFAM binding molecules and anti-TFB2M binding molecules are readily available and/or may be readily produced by methods known in the art, and are further described in the appended Examples. Furthermore, at least one of said primary binding molecules may comprises an oligonucleotide (e.g. a proximity oligonucleotide) as described herein. Furthermore, the kit may comprise at least one secondary binding molecule (e.g. a secondary antibody) comprising an oligonucleotide (e.g. a proximity oligonucleotide). In particular, one secondary binding molecule specifically binds to one of said primary binding molecules and does not bind to the other one of said primary binding molecules (e.g. it may be either anti-anti-TFAM or anti-anti-TFB2M). In particular, a binding molecule comprising an oligonucleotide (e.g. a proximity oligonucleotide), as used herein, may be a binding molecule, e.g. an antibody, to which said oligonucleotide is conjugated or attached, e.g. covalently bound.

In particular, each of said primary binding molecules may comprise an oligonucleotide (e.g. proximity oligonucleotide) and/or can be specifically bound by a secondary binding molecule comprising an oligonucleotide (e.g. proximity oligonucleotide), wherein the two oligonucleotides can form an oligonucleotide template when they are in close proximity to each other, e.g. 40 nm or less apart from each other, and wherein said oligonucleotide template can be amplified and/or extended, as described herein, e.g. in PLA or proxHCR.

Thus, the kit may further comprise a ligase, a polymerase and/or a detection polynucleotide that can be used in a proximity ligation assay (PLA).

Furthermore, the kit may comprise an activator oligonucleotide and at least two HCR amplification hairpin oligonucleotides, i.e. wherein the oligonucleotides comprised in a binding molecule are proximity hairpin nucleotides, and wherein said oligonucleotides can be used for a proximity-dependent initiation of hybridization chain reaction (proxHCR), e.g. as described in Koos (2014), Nature Communications 6, and/or the published patent application US20170009278.

At least one of the binding molecules comprised in the kit may be an antibody, a monobody, or an aptamer, as described herein. Preferably, said at least one binding molecule is an antibody. Preferably, said at least one binding molecule is an antibody conjugated to an oligonucleotide (e.g. proximity oligonucleotide).

Preferably, the kit of the invention may further comprise a brochure or leaflet with instructions for measuring or determining the level of mature and/or active TFAM protein in a sample from a subject or patient as described herein, and/or for carrying out at least one of the inventive methods provided herein, e.g. the inventive method of detecting an abnormal level of mature and/or active TFAM protein in a sample from a patient.

The inventive binding molecule MAT, inventive binding molecule IMM and/or the inventive kit provided herein may be used in an inventive method provided herein, e.g. in a diagnostic and/or prognostic method of the invention, and/or in a drug screening method according to the invention.

Furthermore, the invention relates to the use of the binding molecule MAT, the binding molecule IMM, and/or the inventive kit provided herein for diagnosing the severity of an organ dysfunction and/or prognosing the outcome of a patient suffering from an organ dysfunction, e.g. the use in a prognostic or diagnostic method of the invention.

Furthermore, the invention relates to the use of the binding molecule MAT, the binding molecule IMM, and/or the inventive kit provided herein for determining the level of mature and/or active mitochondrial transcription factor A (TFAM) protein in a sample, e.g. according to the prognostic or diagnostic method of the invention.

In particular, said inventive uses may be in vitro, and/or for an in vitro diagnostic method.

The invention also relates to the use of the binding molecule MAT, the binding molecule IMM, and/or the inventive kit provided herein for non-diagnostic methods practiced on the human or animal body.

Without being bound by theory, at least three potential mechanisms may explain the surprising epiphenomena of the reduced TFAM-TFB2M interaction in septic patients and upon LPS stimulation of cells from healthy subjected, as described herein in the context of the present invention and illustrated in the appended Examples. First, an impaired transport over the outer membrane is a rational explanation. Since, TFAM is a nuclear encoded protein, it needs to go through a complex import mechanism to reach its site of action in the mitochondrion (Prasai (2017), Pathophysiology 24). For the first part of the journey, HSP70 is needed to facilitate proper folding and direct the Pre-TFAM protein (immature TFAM protein) towards the outer mitochondrial membrane via docking at TOM70 followed by release in the translocase TOM40 (Prasai (2017), Pathophysiology 24). Since the release of Pre-TFAM is an energy consuming process, it is plausible that the import of Pre-TRAM is prone to disruptions in conditions associated with ATP depletion like sepsis (Singer (2017), Crit Care 21). In addition, the outer mitochondrial transport proteins TOM40 and TOM70 may contribute to an impaired TFAM import as has been discussed in the context of patients with diabetic retinopathy and neurodegenerative diseases (Santos (2013), Diabetes Metab Res Rev 29; Kimura (2012), J Alzheimers Dis 29).

Second, particularly the transport over the inner mitochondrial membrane using the translocase TIM23 seems to be vulnerable for import interruptions in sepsis (Prasai (2017), Pathophysiology 24). In detail, after successfully passing the outer mitochondrial membrane Pre-TFAM is handed over to the inner translocase TIM23 (Prasai (2017), Pathophysiology 24). Foremost, the mitochondrial membrane potential (A 0) activates TIM23 and drives translocation of the positively charged protein to the mitochondrial matrix (Prasai (2017), Pathophysiology 24). Since sepsis is associated with depolarization of the mitochondrial membrane that also correlates with the disease severity and outcome (Grundler (2014), Crit Care 18, Adrie (2001); Am J Respir Crit Care Med 164), the inventors contemplate that the depolarized membrane potential might constitute the decisive element of the hampered mitochondrial TFAM import.

Third, the TFAM protein could be lost due to an exaggerated degradation that exceeds the maximal imported capacity. When TFAM protein is not bound to mtDNA, it is rapidly degraded by the mitochondrial LON protease (mLP) (Lu (2013), Mol Cell 49). Furthermore, an increased mLP expression significantly reduced TFAM levels and mtDNA copy number, noting mLP as key regulator of TFAM and mtDNA abundance (Lu (2013), Mol Cell 49, Pinti (2016), Biochim Biophys Acta 1857). In this regard, specific LON protease inhibitors may allow to manipulate TFAM and mtDNA level by regulating the activity of Lon protease as already shown in other diseases (Lan (2017), Biosci Rep 37).

Therefore, the invention further relates to a method for identifying a compound which promotes the transport of TFAM protein into mitochondria and/or which promotes the maturation of TFAM protein (i.e. a drug screening method), wherein said method comprises the steps of
a) determining the level (e.g. amount) of mature and/or active TFAM protein in a sample, as described herein, upon addition of a compound to said sample, wherein said sample comprises at least
   (i) TFAM protein and
   (ii) at least one other mitochondrial protein and/or mitochondrial DNA, to which the mature TFAM protein binds specifically, as described herein, preferably TFB2M protein, and preferably
   (iii) mitochondria, in particular wherein the TFAM protein of (i) and the at least one other mitochondrial protein and/or mitochondrial DNA of (ii) are contained in said mitochondria,
b) evaluating whether the level (e.g. amount) of mature and/or active TFAM protein in the sample is elevated (e.g. in the mitochondria) compared to a control sample and/or before addition of the compound, and
c) determining
   (i) that said compound promotes the transport of TFAM protein into mitochondria and/or the maturation of TFAM protein, when the level of mature and/or active TFAM protein is elevated; or
   (ii) that said compound does not promote the transport of TFAM protein into mitochondria and/or the maturation of TFAM protein, when the level of mature and/or active TFAM protein is not elevated.

Furthermore, the inventive drug screening method provided herein may comprise contacting the sample in step a), in particular in the absence of mitochondria, with a solid support to which the TFAM protein of (i) and/or the at least one other mitochondrial protein and/or mitochondrial DNA of (ii) can bind. In particular, capture binding molecules (e.g. antibodies) may be immobilized to such a solid support and mediate the binding of TFAM protein and/or said at least one other mitochondrial protein and/or mitochondrial DNA. Furthermore, for example, inter alia polymers or glass may be coated with streptavidin to which binding molecules (e.g. affinity binders) conjugated to biotin can bind.

In particular, the compound which promotes the transport of TFAM protein into mitochondria and/or the maturation of TFAM protein may be used for treating an organ dysfunction and/or sepsis, as described herein.

In addition, the inventive drug screening method provided herein may further comprise
in step a) determining the level of immature and/or inactive TFAM protein upon addition of said compound to said sample,
in step b) evaluating whether the level of immature and/or inactive TFAM protein is reduced, and
in step c) determining that the compound promotes the transport of TFAM protein into mitochondria and/or the maturation of TFAM protein, when the level of mature and/or active TFAM protein is elevated and the level of immature and/or inactive TFAM protein is reduced.

As regards determining the level of mature and/or active TFAM protein in a sample, the level of mature and/or active TFAM protein, the mature and/or active TFAM protein and the immature and/or inactive TFAM protein, the same applies as is described herein in the context of the diagnostic and/or prognostic methods of the invention.

In particular, the mature and/or active TFAM protein is in the mitochondria and the immature and/or inactive TFAM protein is outside the mitochondria (e.g. in the cytonucleoplasm).

Furthermore, the invention relates to a compound identified by the inventive drug screening method provided herein for use in treating an organ dysfunction and/or sepsis as described herein. In particular, said compound may be an antioxidant as described herein.

The invention is also characterized by the following figures, figure legends and the following non-limiting examples.

### BRIEF DESCRIPTION OF THE FIGURES

FIG. 1. Lipopolysaccharide (LPS) increases extramitochondrial but not intramitochondrial TFAM abundance. LPS stimulation of PBMCs from healthy volunteers before and after 0.5, 4, 24, and 48 hours, respectively. Concentrations of selected cytokines in PBMC cell culture supernatants (a: TNF-α, b: Interleukin-6, c: Interleukin-10); d: PGC-1α (ELISA of nuclear protein extracts) in PBMCs of healthy volunteers; e: TFAM mRNA expression (quantitative polymerase chain reaction) normalized to beta actin in LPS-stimulated PBMCs of healthy volunteers (n-fold change to control). f-h: TFAM protein expression in LPS-stimulated PBMCs from healthy volunteers determined by western blot. The time course of immature TFAM protein expression in cytonucleoplasm was normalized to beta actin (red) and the relative intramitochondrial mature TFAM protein amount was normalized to TNF receptor-associated protein 1 (blue). Values shown are means with corresponding 95% CI (hatched area) of the n-fold change compared to unstimulated PBMCs at baseline. Representative Western Blot images of immature TFAM protein (29 kDa) in the cytonucleoplasm and mature TFAM protein (24 kDa) in the mitochondria are shown in g and h, respectively. The full Western Blot images are shown in FIGS. 3 a and b (corresponding to FIG. 1g) and FIGS. 3 b and c (corresponding to FIG. 1h). Each circle represents an individual experiment; columns with error bars represent means with SD. P values relate to Wilcoxon Test; *p<0.05, **p<0.01, ***p<0.001, and ns designates no statistically significant difference. There were no missing data. Cytokine concentrations were derived from a calibration curve.

FIG. 2. Evaluation of separation of mitochondria and cytonucleoplasm.
a: Representative Western blot of the mitochondrial located voltage-dependent anion-selective channel protein 2 (VDAC2) to evaluate the proper mitochondrial isolation. The cytonucleoplasm is free of mitochondria. Red arrow shows specific line of VADAC2 at ˜30 kDa. WCL: whole cell lysate, M: mitochondria; CNP: cytonucleoplasm. b: Representative Western blot of the mitochondrial located TNF Receptor Associated Protein 1 (TRAP1) to evaluate proper mitochondrial isolation. The cytonucleoplasm is free of mitochondria. Red arrow shows specific line of TRAP1 at ˜75 kDa. WCL: whole cell lysate, M: mitochondria; CNP: cytonucleoplasm.

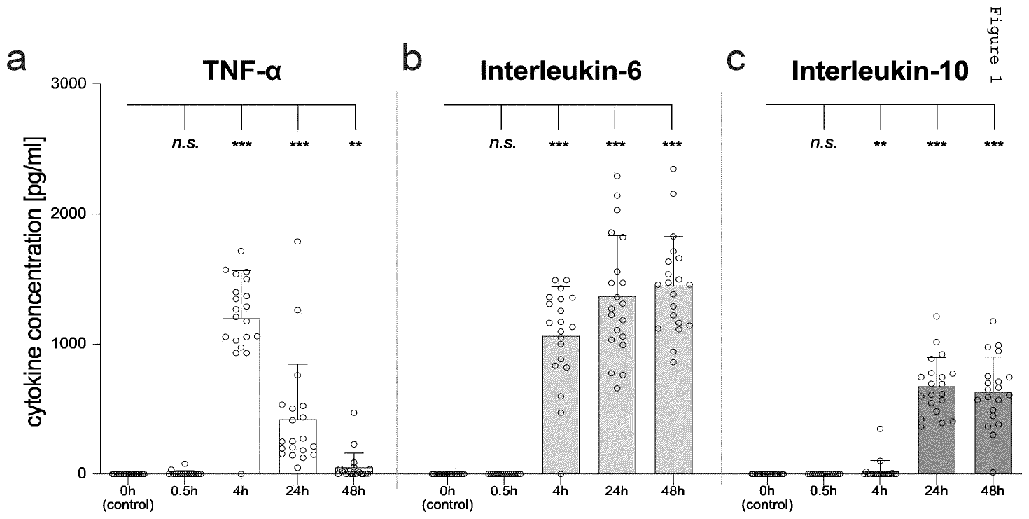

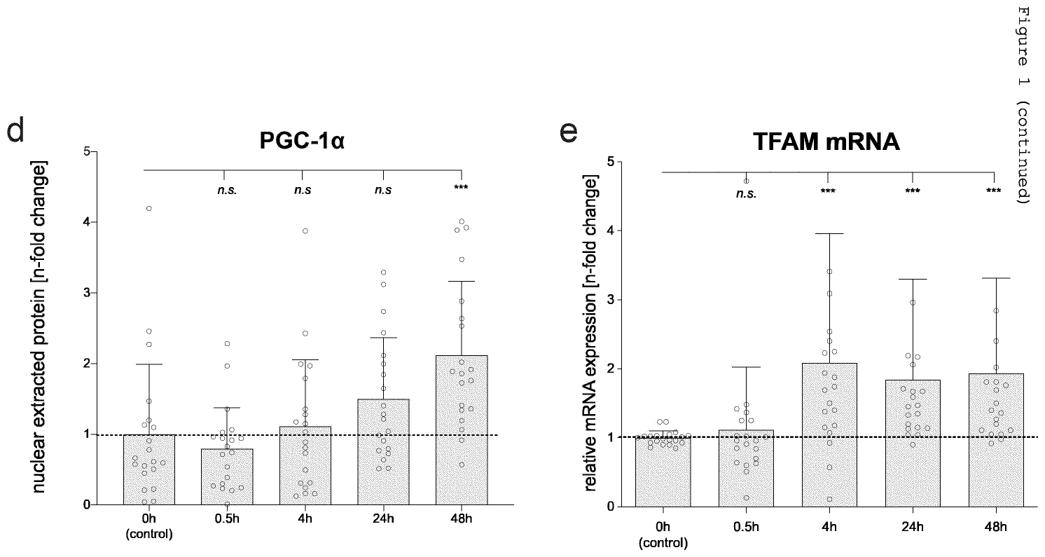

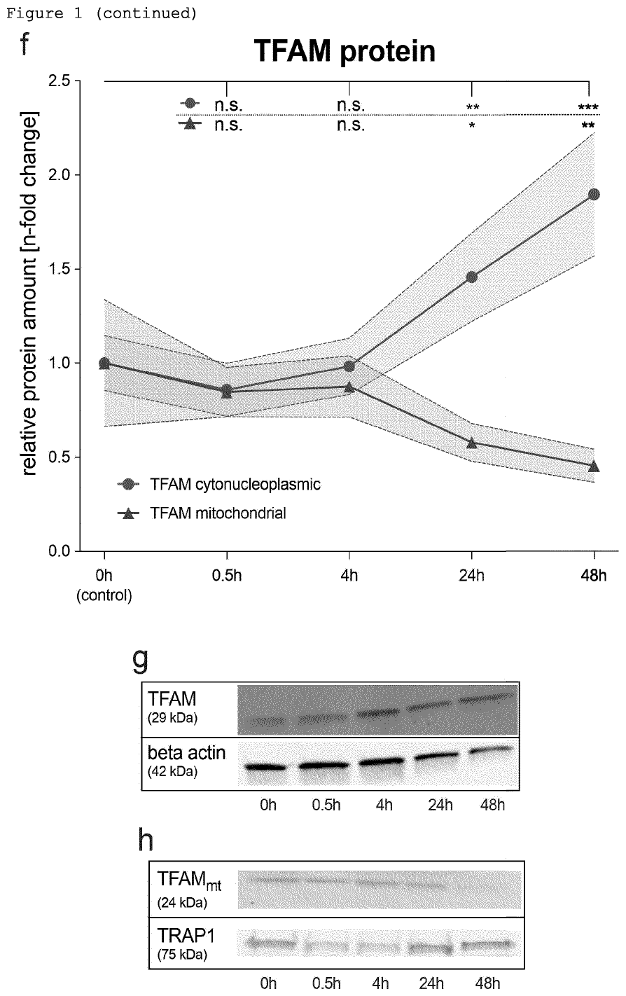

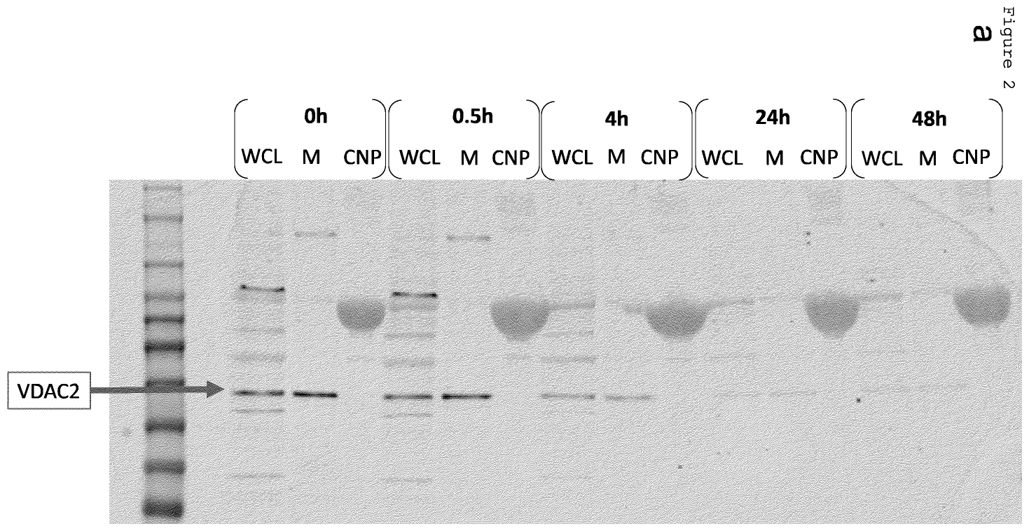

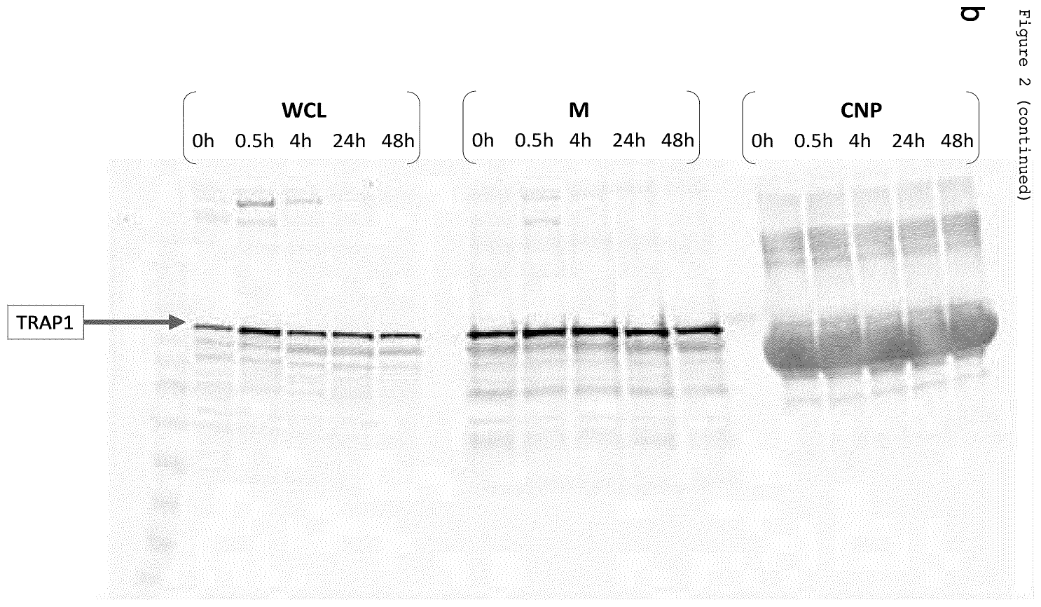

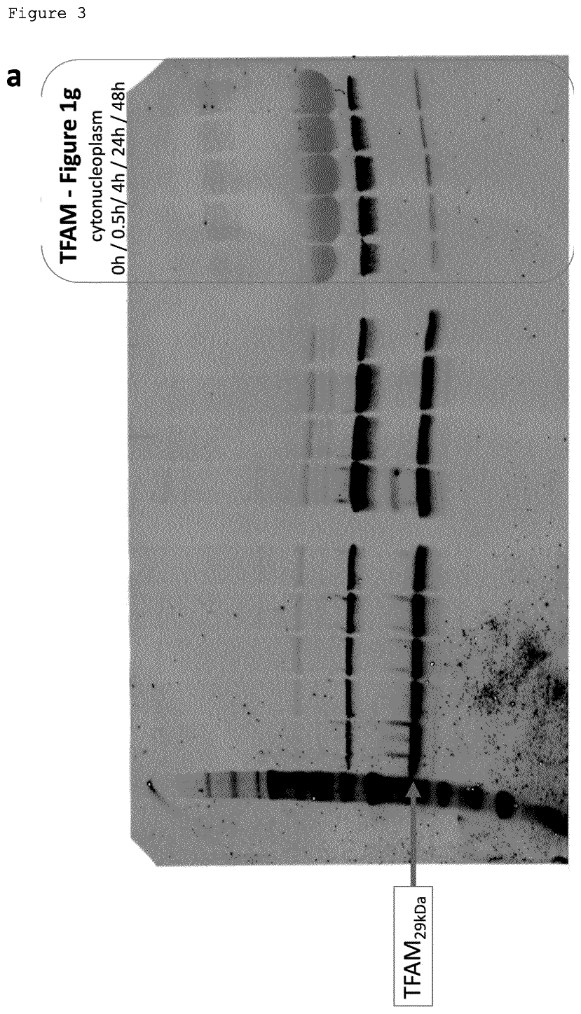

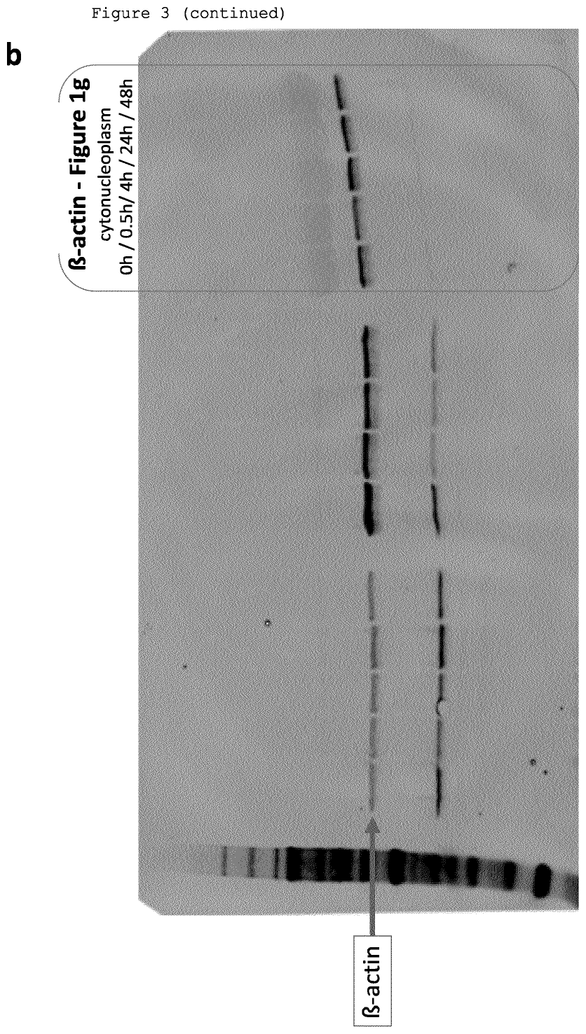

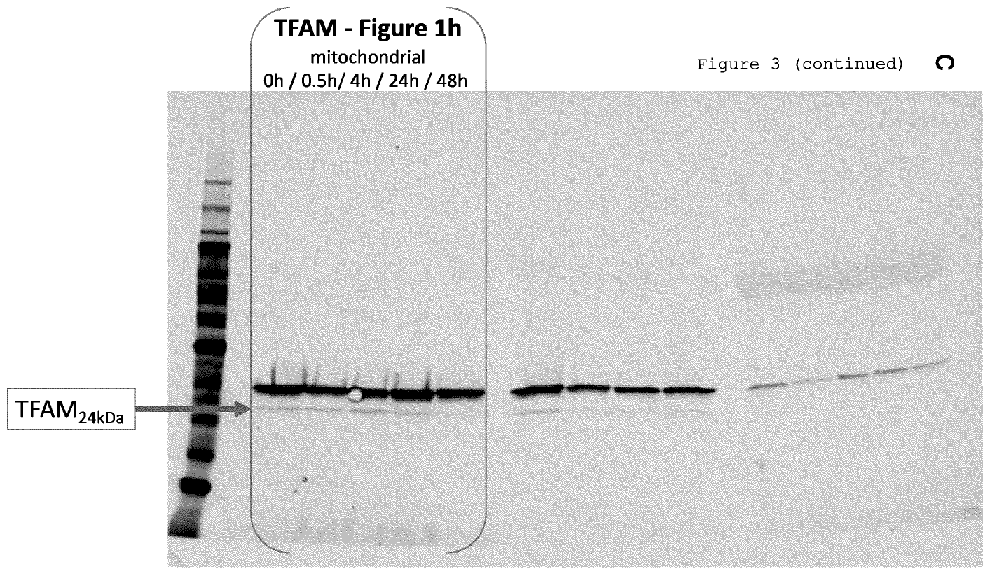

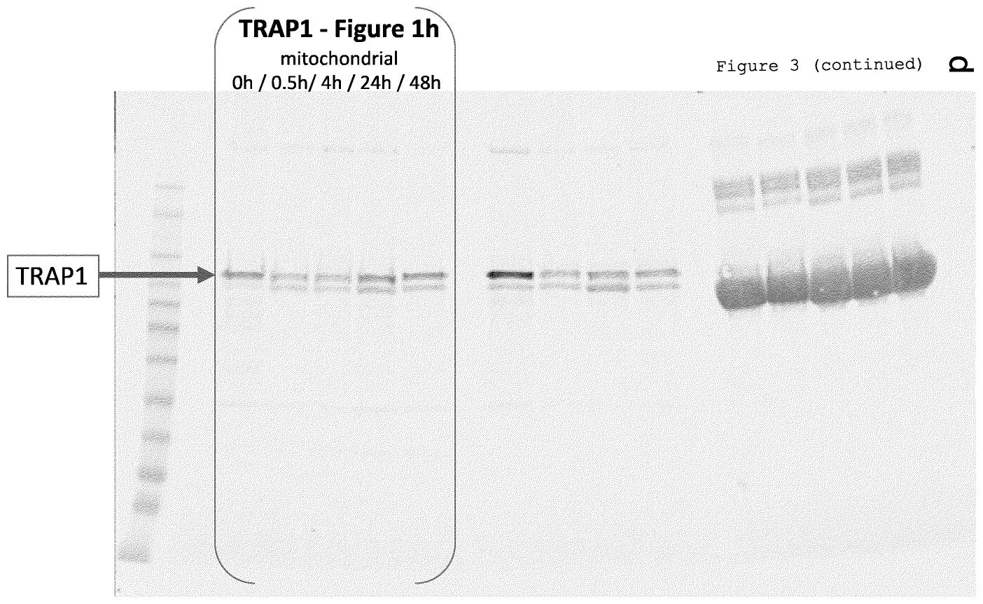

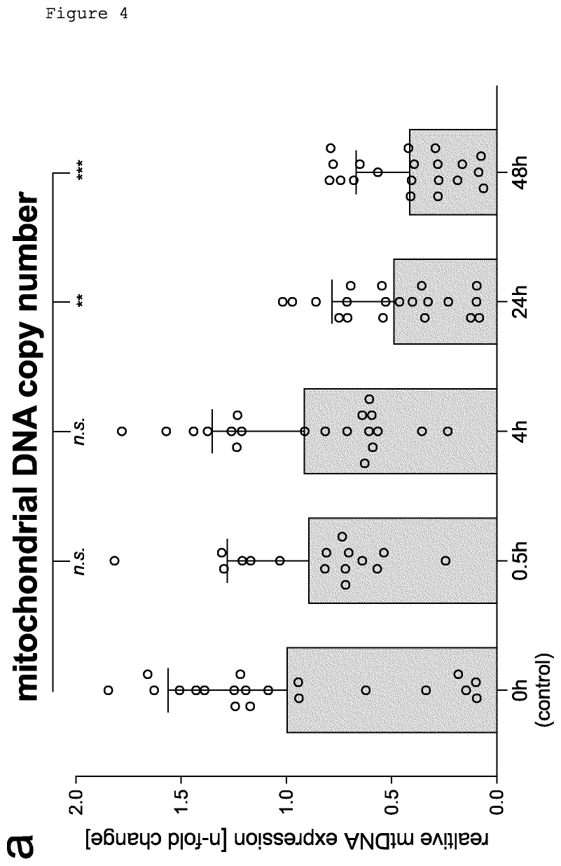

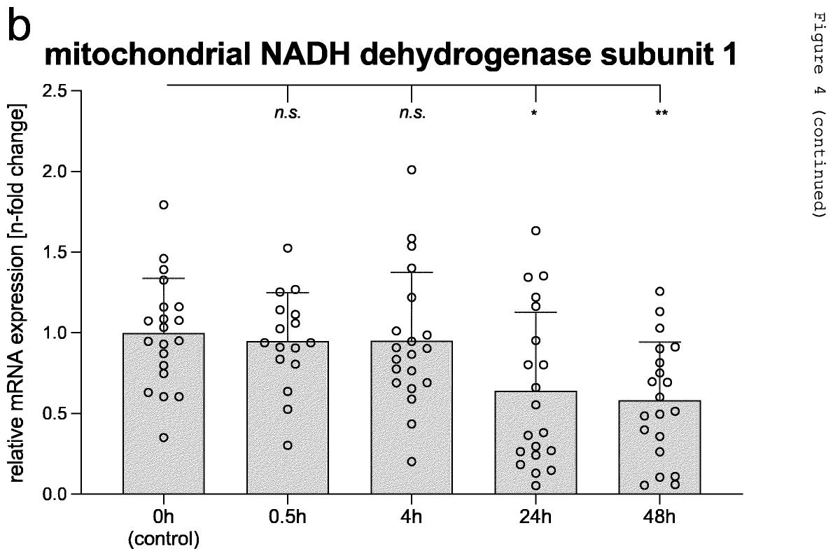

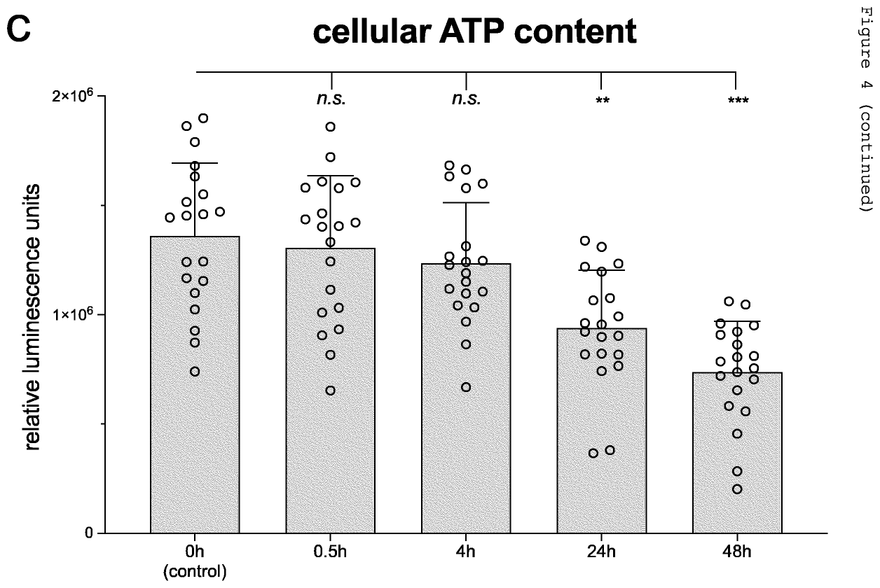

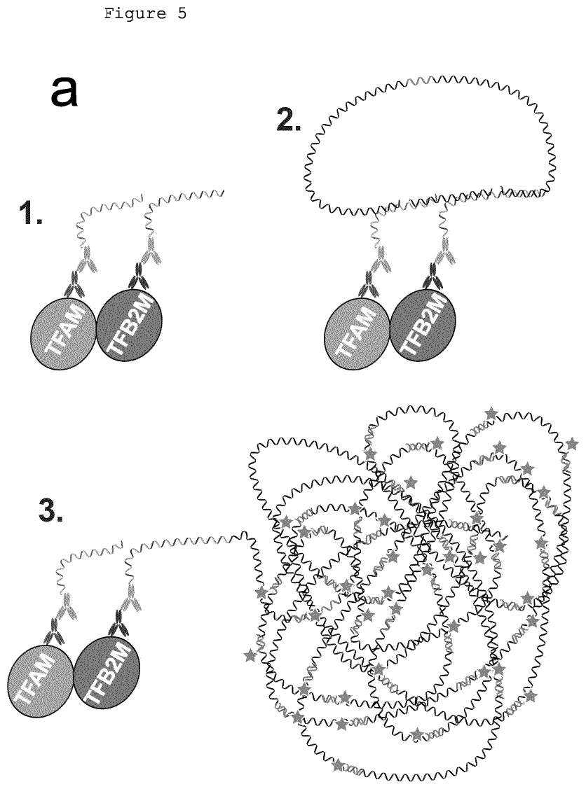

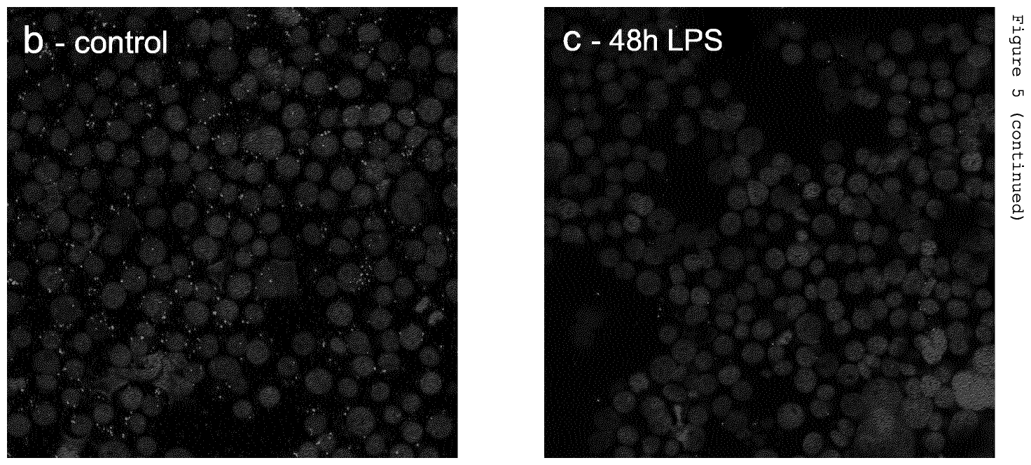

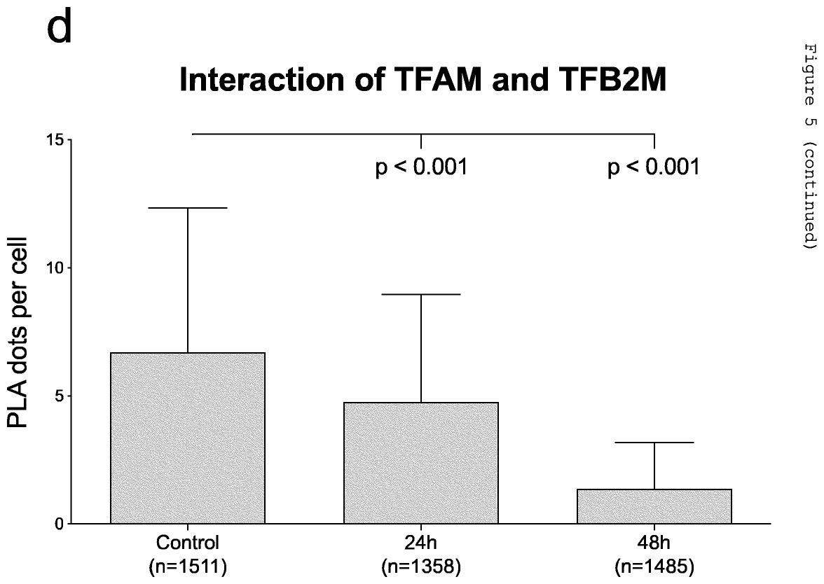

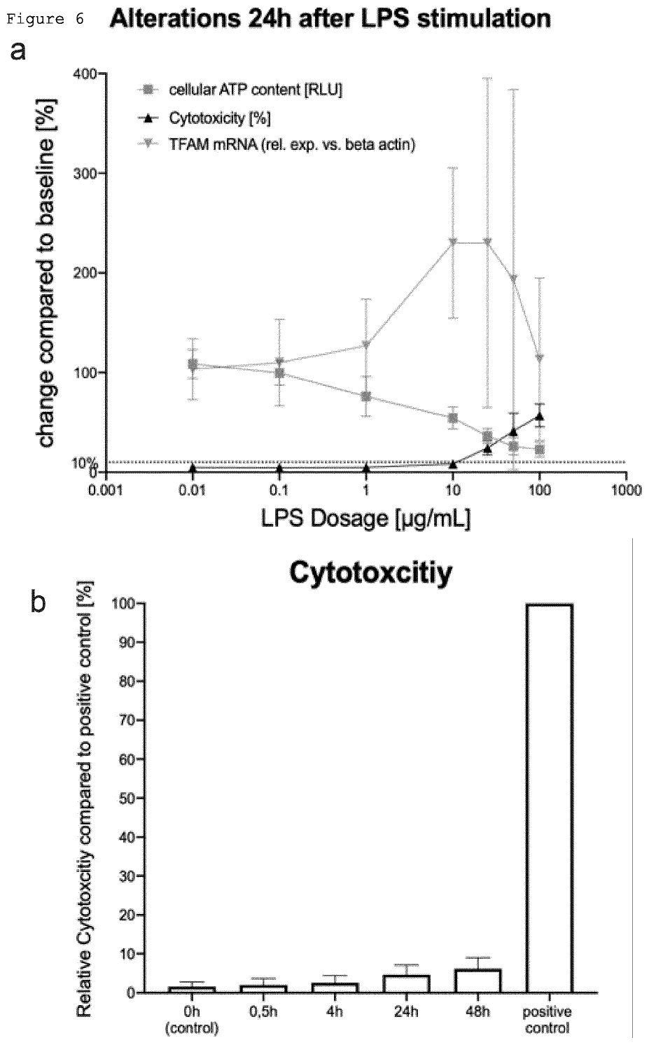

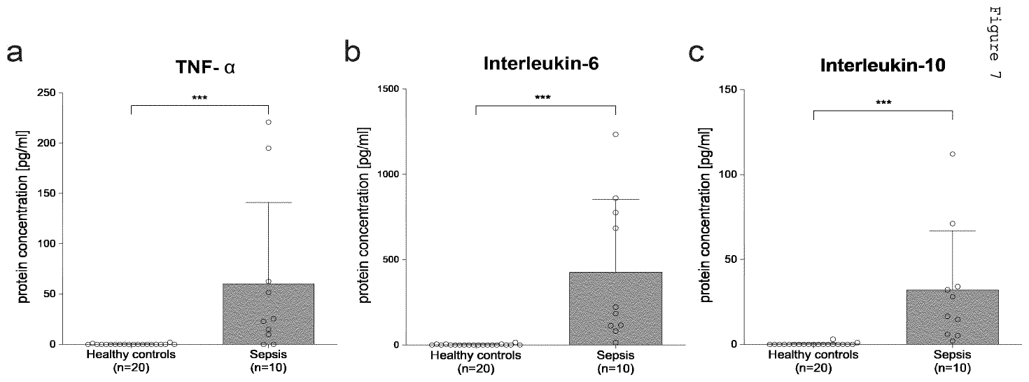

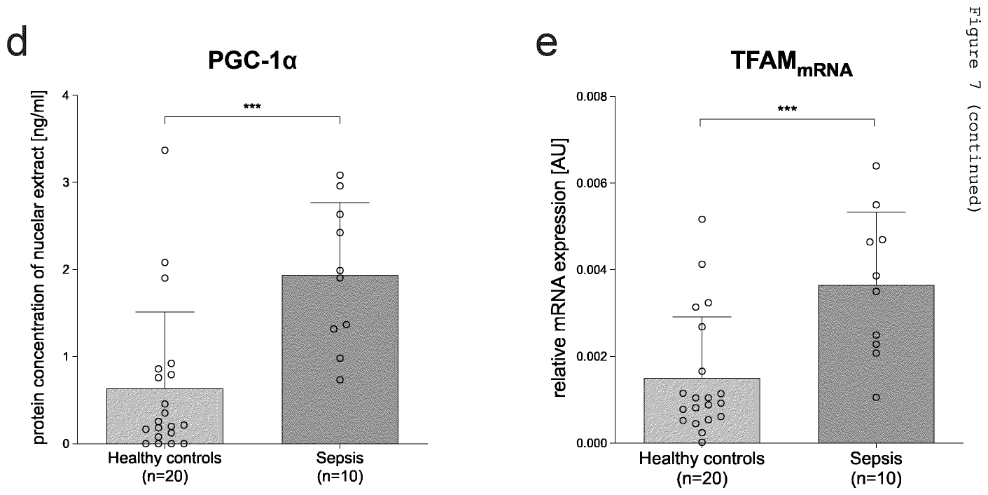

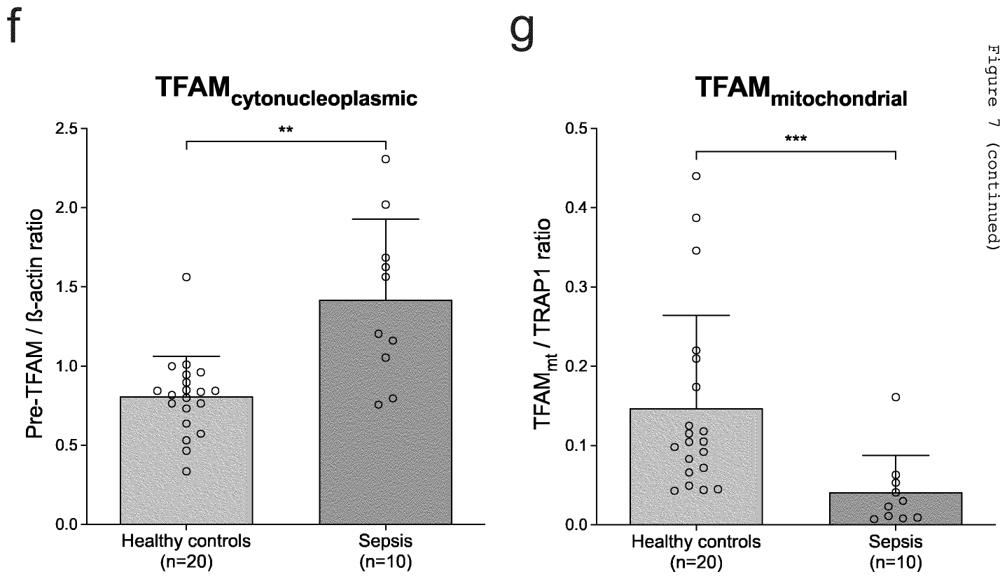

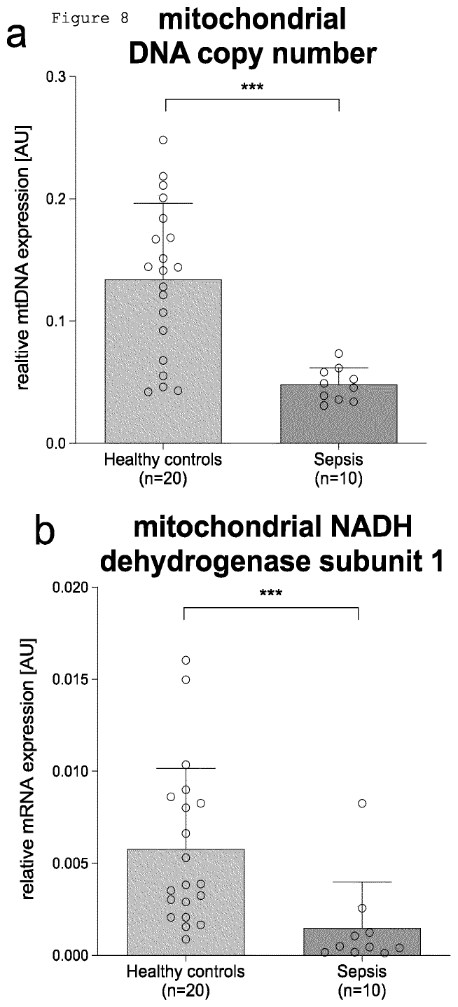

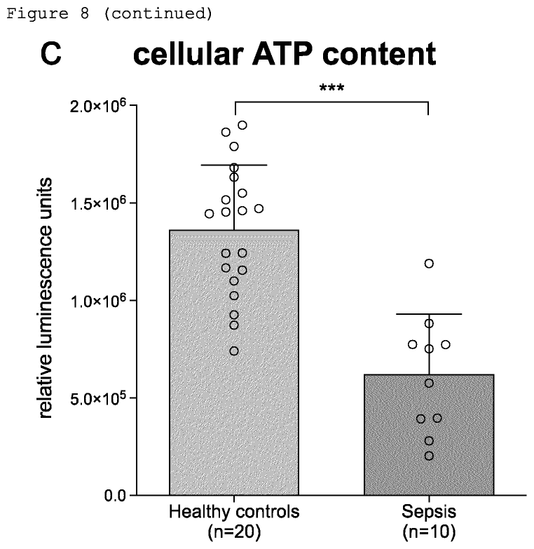

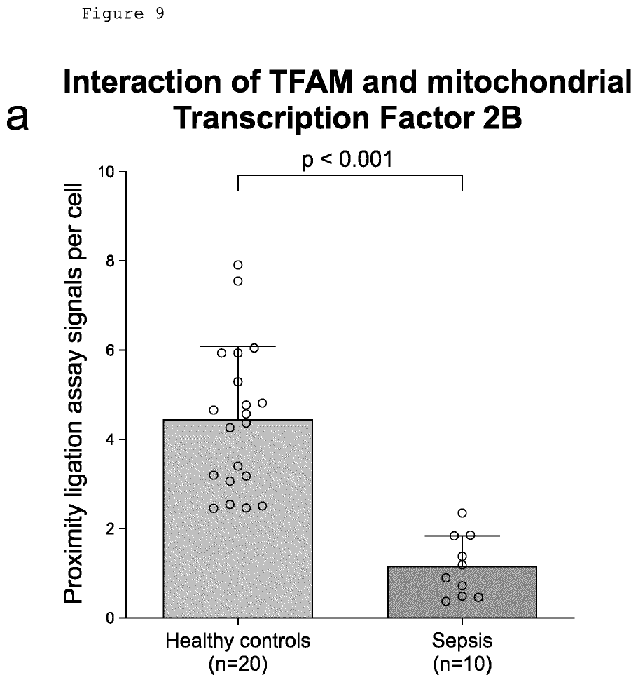

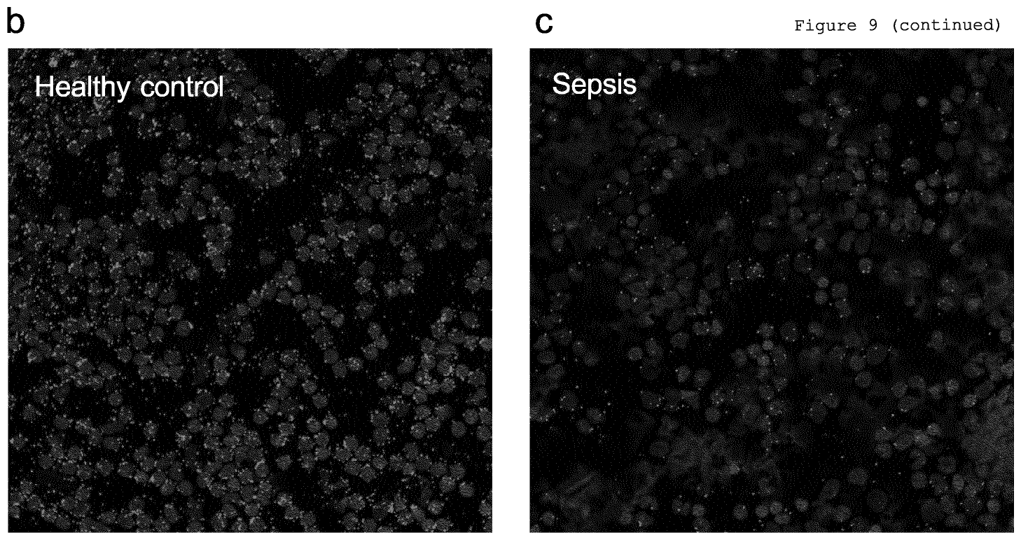

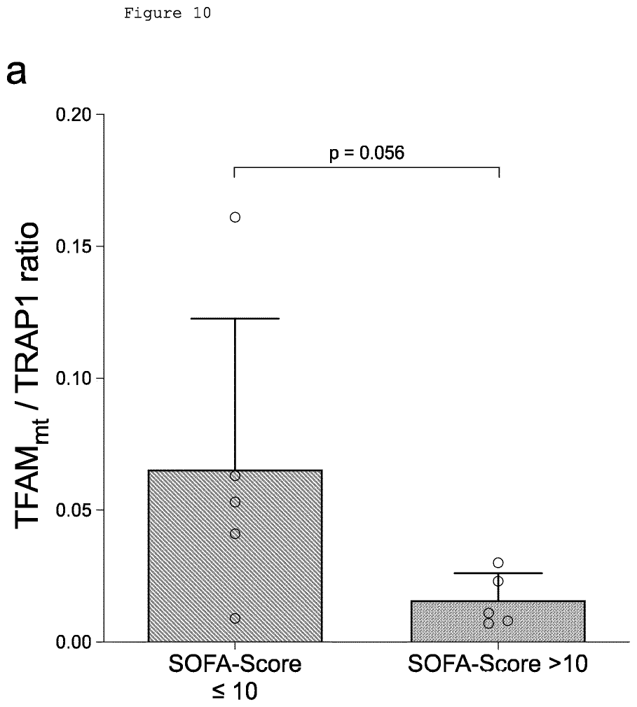

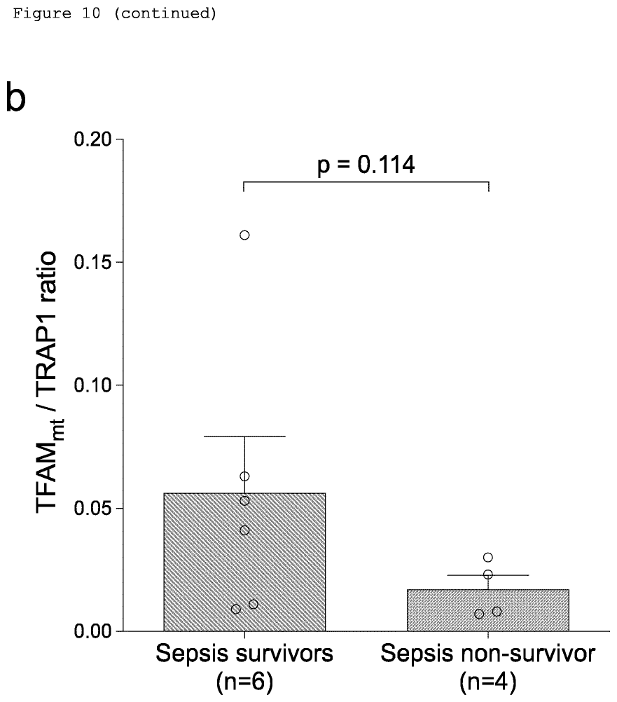

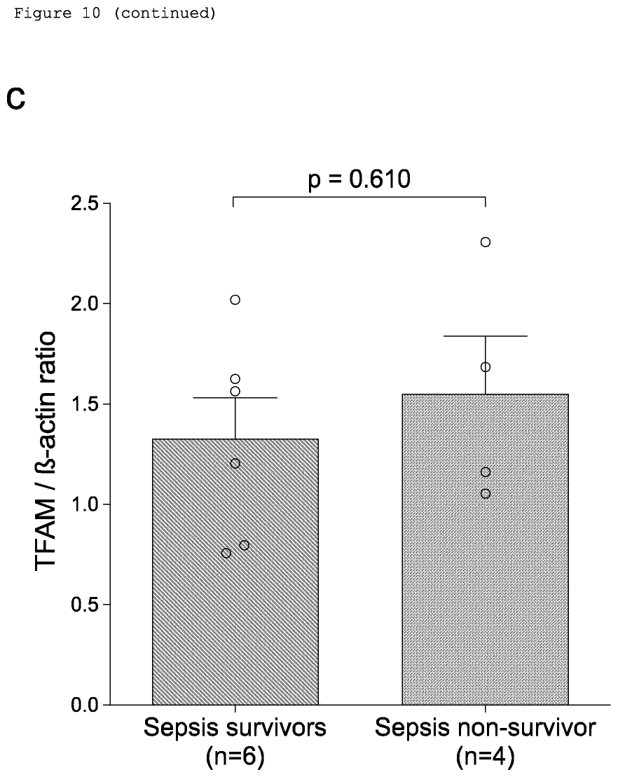

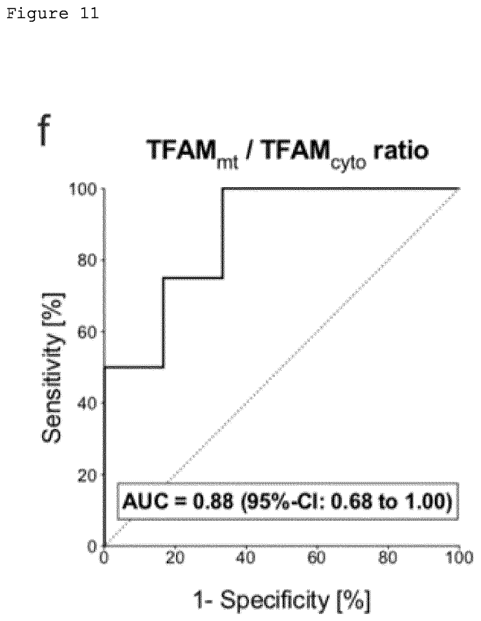

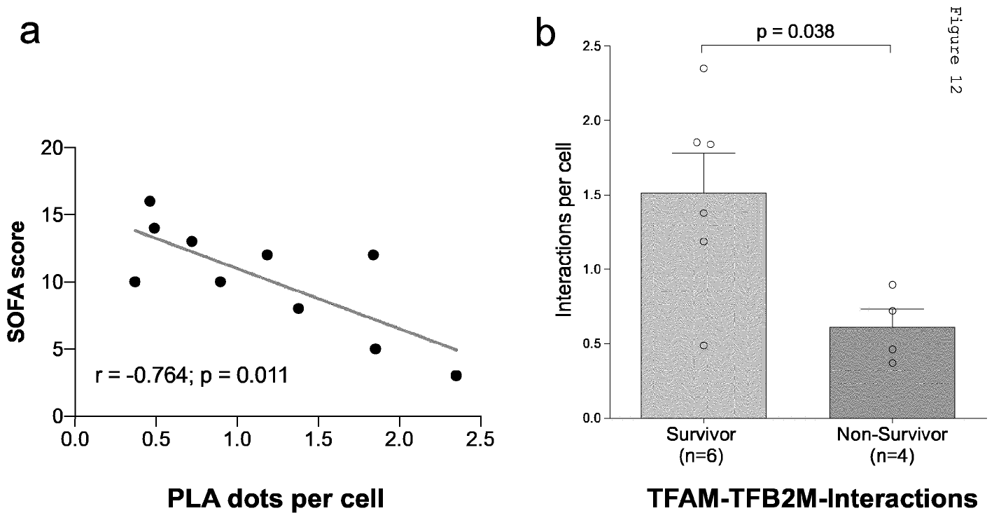

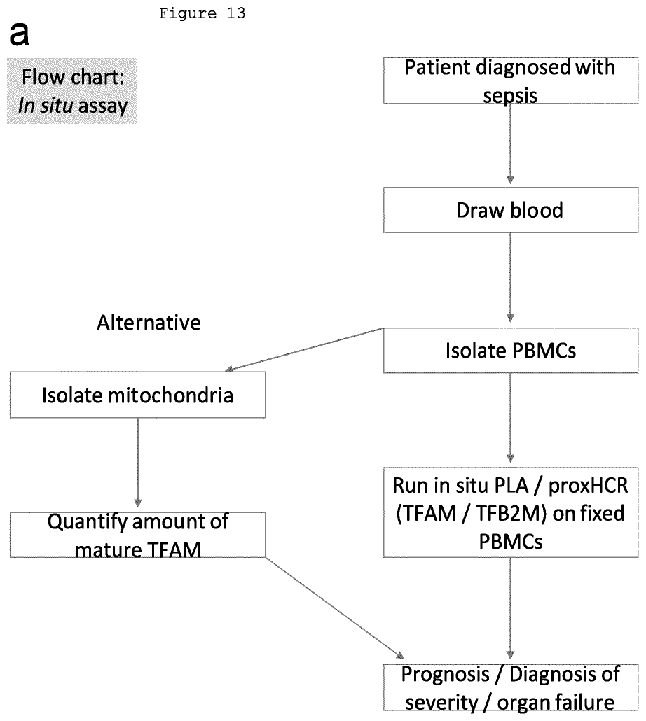

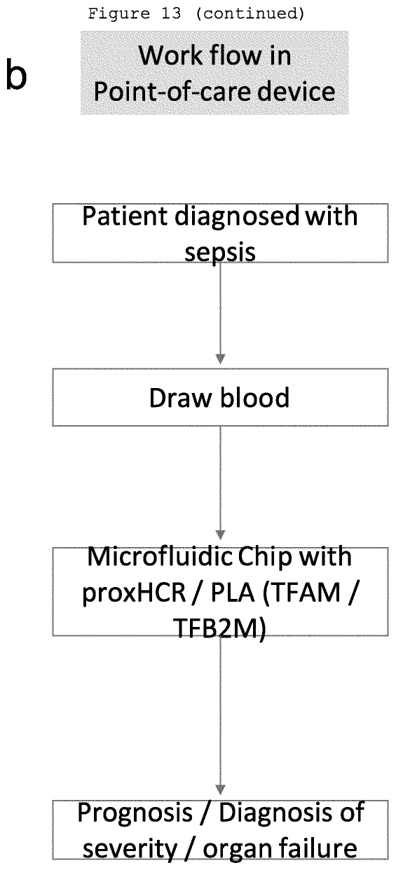

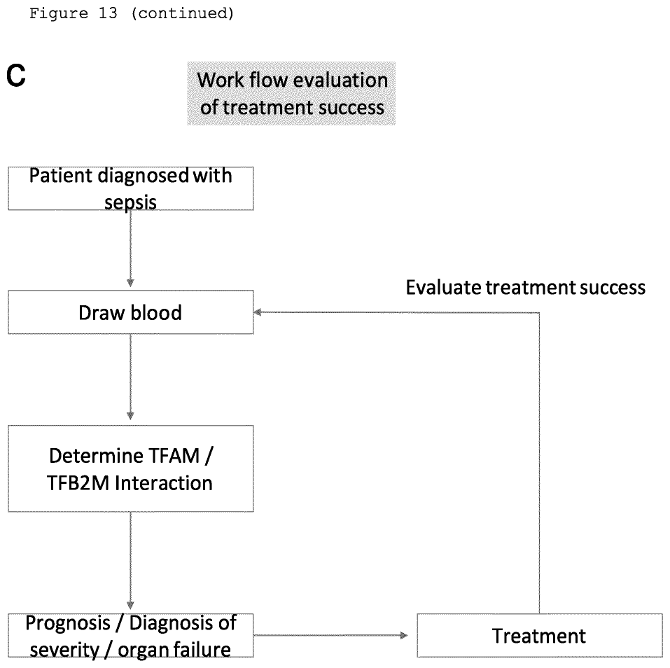

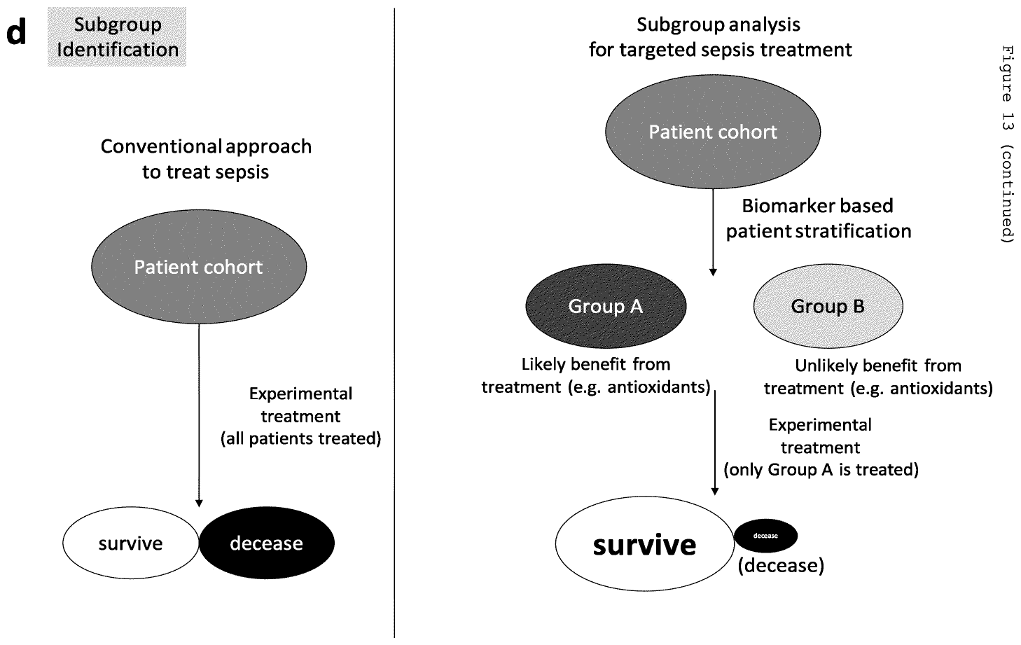

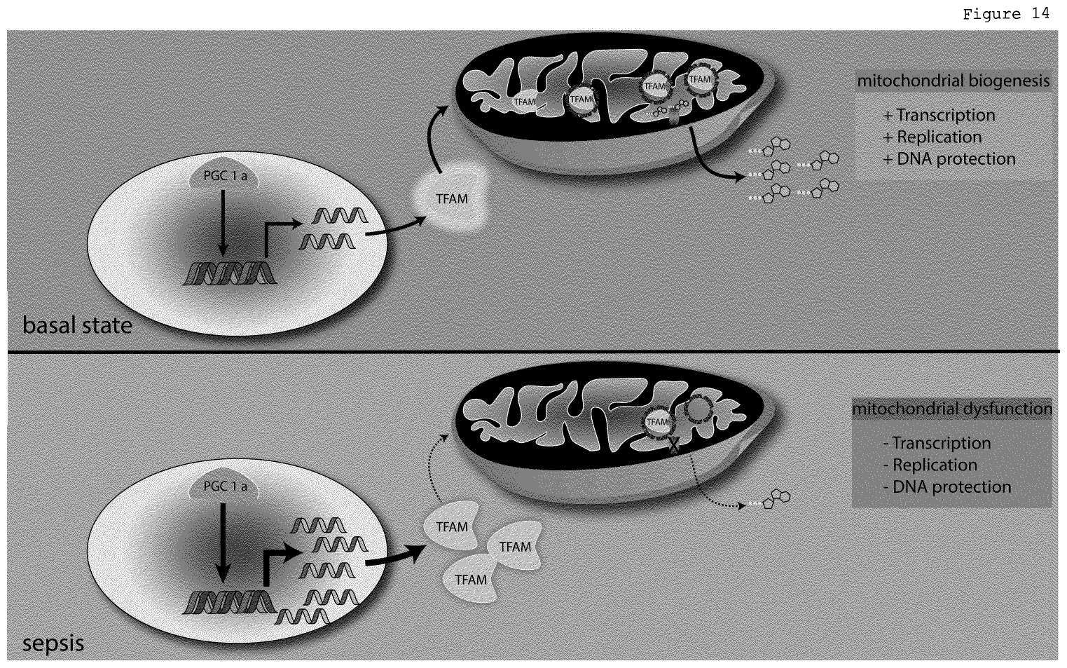

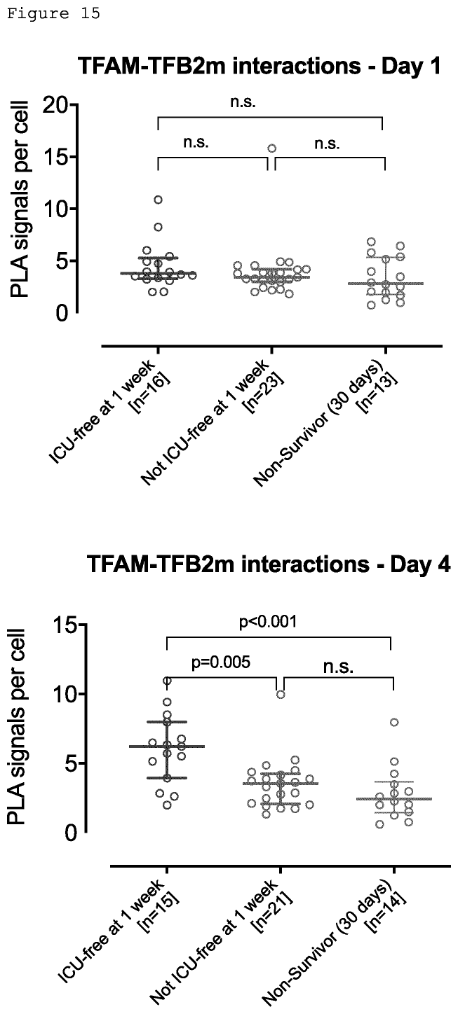

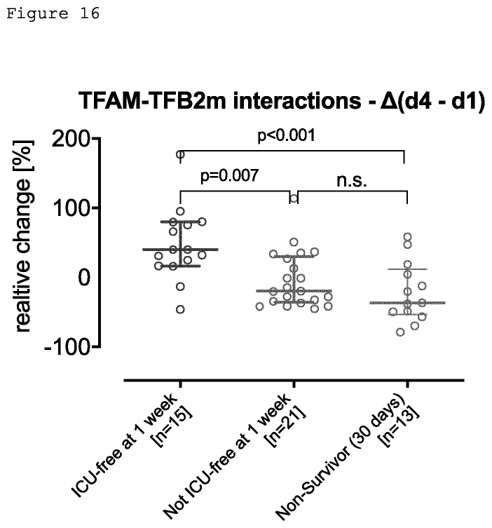

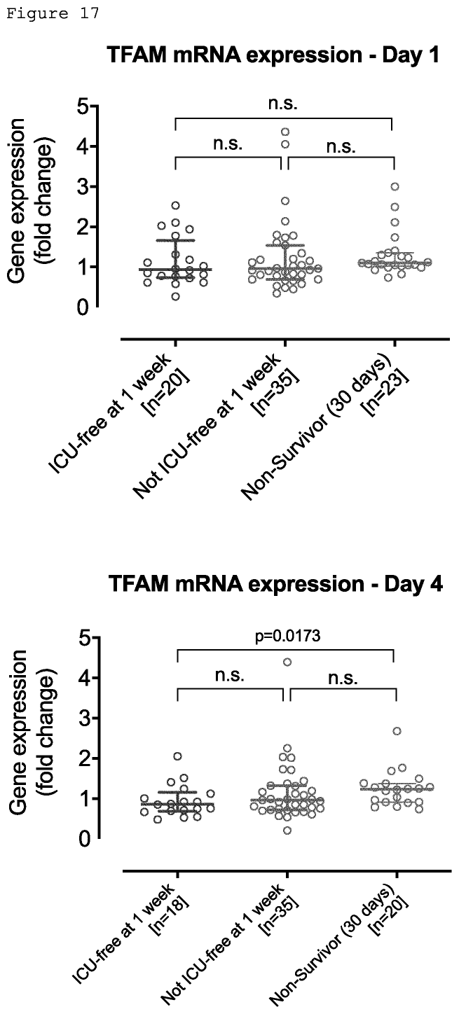

FIG. 3. Western blotting of TFAM and normalizing proteins in mitochondria and cytonucleoplasm.
a and b: Fully illustrated and unprocessed Western Blots of image excerpts from FIG. 1 g; a: TFAM of cytonucleoplasm. Red arrow shows specific line of immature cytonucleoplasmic TFAM at ˜29 kDa; b: β-actin of cytonucleoplasm. The arrow shows specific line of cytonucleoplasmic β-actin ˜42 kDa.
c and d: Fully illustrated and unprocessed Western Blots of image excerpts from FIG. 1 h. c: TFAM of mitochondria. The arrow shows specific line of mature mitochondrial TFAM at ˜24 kDa. The thick band (˜29 kDa) above the arrow refers to the immature TFAM protein in the mitochondria; d: TRAP1 of mitochondria. Red arrow shows specific line of TRAP1 at ˜75 kDa.

FIG. 4. Deterioration of mitochondrial function in LPS-stimulated PBMCs from healthy volunteers. Time course of mitochondrial function indicators (a: mitochondrial DNA copy number, b: mitochondrial NADH dehydrogenase subunit 1, and c: cellular ATP amount) in PBMCs from healthy volunteers before and 0.5, 4, 24, and 48 after LPS stimulation. Specifically, in b: mRNA of mitochondrial encoded mitochondrial NADH dehydrogenase subunit 1; and in c: Cellular ATP was determined using a luciferase-based assay and expressed as relative fluorescent units normalized to 2.25×105 cells per well.

Each circle represents an individual volunteer; columns with errors bar represent means with SD. P-values were determined using the Wilcoxon test *p<0.05, **p<0.01, ***p<0.001, and ns for no statistically significant difference. There were no missing data.

FIG. 5. In-vitro effect of inflammation on the mitochondrial core transcription initiation complex. Proximity ligation assay (PLA) showing mitochondrial interaction of TFAM with TFB2M (known as core transcription initiation complex) in monocytic U937 lymphoma cells treated for 24 h and 48 h with LPS compared to controls without stimulation. a: Principles of proximity ligation assay: 1. After binding of primary antibodies and proximity probes to their respective targets two oligonucleotides are hybridized to the proximity probes. 2. These oligonucleotides are ligated to form a DNA circle, which in turn is amplified yielding a long single stranded DNA molecule. 3. This blob of DNA is then visualized using fluorophore labelled detection oligonucleotides.
b and c: Representative images are shown for unstimulated controls and for 48 h under LPS stimulation. Small bright dots show protein-protein complex formation; nuclei are counterstained with DAPI;
d: Depicted are average numbers of PLA signals per cell (mean with standard deviation) of >150 analyzed cells per condition for each of 5 independent biological replicates. P-values were determined using the Mann-Whitney test.

FIG. 6. Lipopolysaccharide concentration series and cellular cytotoxicity
a: Lipopolysaccharide concentration series. Samples incubated with varying concentrations of LPS were each compared to unstimulated controls (baseline). Relative TFAM mRNA expression (quantitative polymerase chain reaction; compared to beta actin) of PBMCs (triangles pointing downwards). Cellular ATP was determined using a luciferase-based assay and expressed as relative fluorescent units normalized to 2.25×10⁵ cells per well (squares). Cellular cytotoxicity was determined using the CelITox Green assay and expressed as relative cytotoxicity compared to lyzed (by 1% Triton X-100) cells (triangles pointing upwards). Signs with error bars represent mean and SD.
b: Measure of cellular cytotoxicity. To assess the cellular degree of cytotoxicity, the CelITox™ Green Cytotoxicity Assay (Promega, Madison, WI) was performed following the manufacturer's instructions. Briefly, 90 μL of suspended cells (2.5×106 cells/mL) were seeded with 10 μL LPS or 10 μL medium into each well. Positive controls were done using 1% Triton X-100. The plates were equilibrated to room temperature and 100 μL reagent was then added. After 10 min incubation at room temperature luminescence measurements were made on a plate reader (Infinite M200PRO).

FIG. 7. Sepsis is associated with increased abundance of extramitochondrial immature TFAM but diminished abundance of intramitochondrial mature TFAM. Results representing PBMCs from sepsis patients (n=10) sampled within 24 hours after the diagnosis of sepsis compared to healthy controls (n=20). a-c: Concentrations of selected cytokines serum of septic patients and healthy controls (a: TNF-α, b: Interleukin-6, and c: Interleukin-10. d: Peroxisome proliferator-activated receptor gamma coactivator 1-alpha (PGC-1α) level (ELISA of nuclear protein extracts). e: Relative TFAM mRNA expression (quantitative polymerase chain reaction; compared to beta actin) of PBMCs; AU: arbitrary units.
f: Relative amount of immature TFAM protein in cytonucleoplasm (pre-TFAM) normalized to beta actin. g: Relative amount of mature intramitochondrial TFAM protein (ₘₜTFAM) normalized to TNF receptor-associated protein 1 (TRAP1).

Each circle represents an individual volunteer/patient; columns with error bars represent mean and SD. There were no missing data. P-values were determined using the Mann-Whitney test; *p<0.05, **p<0.01, ***p<0.001. Cytokine concentrations pg/mL were derived from a calibration curve.

FIG. 8. Deteriorated mitochondrial function in sepsis patients.

Mitochondrial function indicators (a: mitochondrial DNA copy number, b: mitochondrial NADH dehydrogenase subunit 1, and c: cellular ATP amount) of PBMCs from septic patients (sampled within 24 hours after onset of sepsis compared to healthy controls; AU: arbitrary units. Specifically, in b: mRNA of mitochondrial encoded mitochondrial NADH dehydrogenase subunit 1; and in c: cellular ATP was determined using a luciferase-based assay and expressed as relative fluorescent units normalized to 2.25×105 cells per well.

Each circle represents an individual volunteer or patient; columns with errors bar represent means with SD. P-values were determined using the Mann-Whitney test *p<0.05, **p<0.01, ***p<0.001, and ns for no statistically significant difference. There were no missing data.

FIG. 9. Interaction of TFAM with TFB2M in the human mitochondrial transcription initiation complex is markedly decreased in septic patients.
a: Average numbers of signals per cell (mean±SD) derived from more than 300 analyzed cells for each of the 20 healthy volunteers and 10 septic patients. Each circle represents an individual volunteer/patient. P-values refer to a Mann-Whitney test. There were no missing data.
b and c: Representative images are shown for a healthy volunteer (b) and a sepsis patient (c). Small bright dots reflect protein-protein complex formation; nuclei are counterstained with DAPI.

FIG. 10. The amount of mature TFAM in mitochondria, but not the amount of immature TFAM in the cytonucleoplasm, is associated with the SOFA score and survival of septic patients.
a: Mature mitochondrial TFAM protein amount stratified for the SOFA-score. b: Mitochondrial TFAM protein amount stratified regarding 30 day survival. c: Cytonucleoplasmic immature TFAM protein amount stratified regarding 30 day survival.

The amount of immature TFAM protein in cytonucleoplasm was normalized to beta actin. The amount of mature intramitochondrial TFAM protein (TFAMₘₜ) was normalized to TNF receptor-associated protein 1 (TRAP1).

Each circle represents an individual patient; columns with errors bar represent means with SD. P-values were determined using the Wilcoxon test. There were no missing data.

FIG. 11. Area under the curve (AUC) and 30-day survival vs. non-survival

Receiver operating characteristics using the ratio of the TRAP-normalized amount of mature mitochondrial TFAM protein over the β-actin normalized amount of cytonucleoplasmic TFAM protein in respect to 30-day mortality.

FIG. 12. The number of interactions of TFAM with TFB2M is associated with the SOFA score and survival of septic patients.
a: Number of TFAM/TFB2M interactions per cell correlated well with SOFA score in septic patients (r=−0.764, p=0.011; n=10). b: Average numbers of PLA signals per cell (mean±SEM) derived from more than 300 analyzed cells for each of 10 septic patients. Each circle represents an individual patient. Patents were stratified regarding 30-day survival. p-values refer to a Mann-Whitney test.

FIG. 13. Exemplary flow charts for diagnosing the severity and/or outcome of an organ dysfunction, evaluating the treatment success, and/or indicating at certain treatment based on the diagnosis.
a: In situ assay: Cells are fixed to a slide and the interaction of TFAM with TFB2M is quantified using a Microscope. b: Point-of-case device (POC): A solid phase assay may be run. Specifically, cells may be lysed and the interaction of TFAM with TFB2M may be quantified in lysate by means of horseradish peroxidase (HRP) or related. c: Evaluation of treatment success. d: Identification of subgroups of septic patients by the mature/active TFAM biomarker and experimental treatment of a specific subgroup.

FIG. 14. Graphical summary of findings. After induction of mitochondrial biogenesis TFAM is transcribed, translated and imported into the mitochondrion, where it facilitates multiple functions involved in mitochondrial recovery. During sepsis the transcription and translation of TFAM is strongly enhanced, but the concentration of mature mitochondrial TFAM is severely reduced. Consequently, mitochondria fail to recover from dysfunction in sepsis.

FIG. 15. The number of interactions of TFAM with TFB2M indicates the severity of sepsis and is predictive of the outcome of sepsis. TFAM-TFB2M interactions were measured at day 1 and day 4 in a group of patients, which suffered from sepsis at a similar degree of severity described by a comparable SOFA score at day 1 (in contrast to FIG. 12, where the patients represent a heterogenous cohort of septic patients). Day 1 refers to the day of the diagnosis of sepsis. At day 4, all analyzed patients were still in the hospital. Patients were stratified into three groups: 1) ICU-free at 1 week (and surviving for at least 30 days), 2) Not ICT-free at 1 week (and surviving for at least 30 days), or 3) Non-Survivor (death within 30 days). The interaction between TFAM and TFB2M on day 1 (top panel) did not show significant differences between the three groups. The interaction between TFAM and TFB2M on day 4 (bottom panel) showed a significant difference between the fast recovery group (ICU-free at 1 week) and the slow recovery group (Not ICT-free at 1 week). In addition, the fast recovery group showed significantly higher interactions on day 4 than the group of the patients who succumbed to sepsis (Non-Survivors).

FIG. 16. Repeated measurement of the number of interactions of TFAM with TFB2M allows to monitor the course of sepsis. TFAM-TFB2M interaction data, septic patients, and patient groups are the same as in FIG. 15. The relative change of the interaction between day 4 and day 1 showed strong and significant differences between the fast recovery group (ICU-free at 1 week) and the other two groups (Not ICT-free at 1 week or Non-Survivors).

FIG. 17. TFAM mRNA is a worse indicator of the severity of sepsis and a worse predictor of the outcome of the sepsis than interaction of TFAM with TFB2M. TFAM mRNA levels were measured by qPCR at day 1 and day 4 in a group of patients, which suffered from sepsis at a similar degree of severity (comparable SOFA score) at day 1. The same patients analyzed in FIGS. 15 and 16 were included in this group. Day 1 refers to the day of the diagnosis of sepsis. At day 4, all analyzed patients were still in the hospital. Patients were stratified into three groups as in FIG. 15. Expression of TFAM mRNA at day 1 (top panel) showed no significant differences between patients that were free of ICU within one week (fast recovery group), patients that needed more than one week to recover (slow recovery group; Not ICT-free at 1 week) and patients that succumbed within 30 days (Non-Survivors). Expression of TFAM mRNA at day 4 did not show a significant difference between the fast recovery group and the slow recovery group. There was a small statistically significant difference between the fast recovery group (ICT-free at 1 week) and the patients that died (Non-Survivors). The variation of the mRNA data was much greater, and the predictive power much lower, compared to the TFAM-TFB2M protein interaction data shown in FIG. 15.

### EXAMPLES

Methods and materials are described herein for use in the present disclosure, other, suitable methods and materials known in the art can also be used. The materials, methods, and examples are illustrative only and not intended to be limiting.

### Example 1. LPS Stimulation of PBMCs from Healthy Individuals Elicits an Inflammatory Response and Promotes Mitochondrial Biogenesis but Leads to a Reduced Amount of Mature TFAM Protein in Mitochondria and Mitochondrial Dysfunction

### Materials and Methods

### Study Design and Oversight

The inventors conducted a prospective, observational, single-center, in vitro and in vivo study registered in the German clinical trials database (DRKS00015619) prior to first patient enrollment. The Ethics Committee of the Medical Faculty of the Ruhr-University of Bochum (protocol no. #18-6257) reviewed and approved the study and written informed consent was obtained from healthy subjects and patients or their guardians, as appropriate. This study was conducted in accordance with the revised Declaration of Helsinki, good clinical practice guidelines, and local regulatory requirements.

### Healthy Subjects

The inventors recruited twenty healthy subjects from the Medical Faculty of the Ruhr-University Bochum between October 10 and Dec. 21, 2018 who were free from infection for at least 4 weeks prior to study participation. The 20 healthy volunteers consisted of 9 females and 11 males with a mean age of 39 years±9.

### Isolation of Peripheral Blood Mononuclear Cells (PBMCs)

Blood was drawn from a peripheral vein and peripheral blood mononuclear cells (PBMCs) were isolated using a density gradient centrifugation protocol (Ficoll Paque solution, GE Healthcare Bio Science AB, Uppsala Sweden). Briefly, cells were centrifuged in Ficoll Paque solution, forming a PBMC-rich layer that was collected. The inventors then resuspended the isolated cells in full RPMI 1640 medium (Invitrogen, Carlsbad, CA) containing 10% fetal calf serum (FCS) (Biochrom AG, Berlin, Germany), 100 U/mL penicillin plus 100 μg/mL streptomycin (both Invitrogen), and held at 37° C. in a humidified atmosphere containing 5% CO2 until further use.

### Stimulation of PBMCs

PBMCs of healthy subjects were seeded at a density of 2×10⁷ cells per well and incubated in a humidified incubation chamber (37° C.; 5% CO2 in air) with or without 10 μg/mL LPS (see FIG. 6a for optimal LPS dosage; Escherichia coli type 0111:B4; L4391, Sigma-Aldrich, St. Louis, MI). Serial in vitro measurements were performed at baseline prior to LPS stimulation and at 0.5, 4, 24, and 48 h.

### Cytokine Concentrations

Supernatant of PBMCs was collected and used for quantifying the cytokines TNF-α, interleukin-6, and interleukin-10 utilizing appropriate human ELISA kits (all BioLegend, San Diego, CA) according to the manufacturers' instructions. Briefly, samples were incubated in precoated ELISA plates for 2.5 h at room temperature. After addition of substrate, the ELISA plates were incubated for 10-30 minutes, and the intensity of the colored product was then measured with a plate reader (Infinite M200PRO, Tecan Group AG; Månnedorf, Switzerland). The concentration of each cytokine was derived by applying respective calibration standard curves.

### Isolation of Mitochondria

To assess and compare intramitochondrial TFAM concentrations to their mitochondria-free cytonucleoplasm, the mitochondria were isolated for each measurement, adapted from the protocol in Argan (1983), J Biol Chem 258. Briefly, the supernatant of the PBMCs was first collected for quantification of TNF-α, IL-6, and IL-10 as described above. The cells were then osmotically swelled and mechanically shredded (homogenized) to release the mitochondria. The mitochondria were then separated from the cytonucleoplasm and cellular debris by different centrifugation steps. Mitochondria were then lysed and protein was isolated. In particular, cells were centrifuged at 800 g for 5 minutes, resuspended, and homogenized in Solution A (2 mg/ml BSA and 0.5 mM PMSF). The homogenate was then centrifuged twice. The pellet containing the mitochondria was resuspended in Solution B (20 mM HEPES KOH buffer pH 7.6, 220 mM mannitol, 70 mM sucrose buffer, 1 mM EDTA, and 0.5 mM PMSF). The resuspended homogenate was then centrifuged to obtain a mitochondrial fraction pellet which was dissolved in RIPA buffer including Halt Protease & Phosphatase Inhibitor Cocktail (ThermoFisher Scientific, Waltham, MA), and frozen until subsequent analyses. The quality of the mitochondrial isolation procedure was validated as shown in FIG. 2.

### Intramitochondrial and Extramitochondrial TFAM Protein as Determined by Western Blot

Western blot was used to assess intramitochondrial and extramitochondrial (=cytonucleoplasmic) TFAM protein levels. Equal amounts of protein were separated on a 4-20% polyacrylamide gradient gel (Bio-Rad, Hercules, CA) and subsequently blotted onto a nitrocellulose membranes (Bio-Rad). Nonspecific binding was blocked by incubation with blocking buffer (5% skim milk and PBS) for 1 hour at room temperature. Subsequently, the membranes were probed with the primary antibodies against TFAM (1:200; sc-376672, Santa Cruz Biotechnology, Dallas, TX), TNF receptor-associated protein 1 (1:500; Sigma-Aldrich), and beta actin (1:10.000, Millipore, Temecula, CA) for five hours at room temperature. Then, unbound primary antibodies were removed. After incubation of secondary antibodies (1:10.000; Goat-anti-mouse, IRDye680RD, and 1:10.000; donkey-anti-rabbit, IRDye 800cw, both Li-cor Biosciences, Lincoln, NE) for one hour at room temperature, protein bands were visualized with an Odyssey Scanner (Li-cor Biosciences) and the densitometry was determined using Image J 2.0 software (National Institutes of Health, Bethesda, MD). TRAP1 was used for normalization of mature mitochondrial TFAM since the TRAP1 concentration proved to be stable in mitochondria during LPS stimulation (FIG. 2b), in contrast to VDAC2 (FIG. 2a). Beta actin was used for normalization of immature TFAM in the cytonucleoplasm.

### Quantification of Peroxisome Proliferator-Activated Receptor Gamma Coactivator 1-Alpha (PGC-1α) Protein

For PGC-1α, the master regulator of mitochondrial biogenesis (see, e.g. Suliman (2004), Cardiovasc Res 64), quantification of a separate nuclear protein extraction was performed where cells were centrifuged at 4000 g for 7 minutes and the pellet then resuspended in Pre-Extraction Buffer (Abcam, Cambridge, UK) allowing the cells to swell on ice. After vortexing and further centrifugation, the pellet was dissolved in Complete Lysis Buffer (Active Motif, Carlsbad, CA). The lysate was then sonicated to ensure complete lysis and then centrifuged at 13.000 g. The supernatant containing the nuclear proteins was used for further analysis. The concentrations of PGC-1α, were measured using a dedicated human ELISA kit (Wuhan EIAab Science Co, Wuhan, China) according to the manufacturers' instructions.

### Quantification of the Expression of TFAM, Mitochondrial NADH Dehydrogenase Subunit 1, and Mitochondrial DNA

To assess the gene products by quantitative polymerase chain reaction, total DNA and RNA was extracted from PBMCs using the QIAamp and RNeasy kits respectively, according to the manufacturer's instructions (QIAGEN, Hilden, Germany). In mRNA samples, the purified RNA was reverse transcribed into complementary DNA using the QuantiTect Reverse Transcription Kit (QIAGEN). Polymerase chain reaction was performed in duplicate using the GoTaq1 qPCR Master Mix (Promega) and specific primers (see Table 1) on a CFX Connect Real-Time System (Bio-Rad Labs). Relative mRNA expression was calculated after normalization using beta actin and ribosomal protein lateral stalk subunit P1 as internal controls using the 2^{-ΔΔCT} method (Rao (2013), Biostat Bioinforma Biomath 3).

Mitochondrial DNA copy number was quantified as the ratio of DNA products of mitochondrial NADH dehydrogenase subunit 1 normalized to ribosomal 18S-RNA serving as an internal control (see Table 1 for the primers) using the 2^{--ΔΔCT} method (Kraft (2019), Crit Care Med 47).

All SYBR green-based quantitative polymerase chain reaction reactions were routinely checked for specificity by analyzing the melting curves of the products subsequent to the reaction.

TABLE 1
Oligonucleotide pairs used for quantitative
polymerase chain reaction

| | |
|---|---|
| Oligonucleotide name | Sequence |

mRNA (c-DNA) targets

| | |
|---|---|
| PGC-1α forward | 5′-TGAACTGAGGGACAGTG |
| | ATTTC-3′ |
| PGC-1α reverse | 5′-CCCAAGGGTAGCTCAGT |
| | TTATC-3′ |
| TFAM forward | 5′-CTCAGAACCCAGATGCA |
| | AA-3′ |
| TFAM reverse | 5′-GCCACTCCGCCCTATAA |
| | -3′ |
| Beta actin forward | 5′-CATGTACGTTGCTATCC |
| | AGGC-3′ |
| Beta actin reverse | 5′-CTCCTTAATGTCACGCA |
| | CGAT-3′ |
| Mitochondrial NADH | 5′-TGGGTACAATGAGGAGT |
| dehydrogenase | AGG-3′ |
| subunit 1 forward | |
| Mitochondrial NADH | 5′-GGAGTAATCCAGGTCGG |
| dehydrogenase | T-3′ |
| subunit 1 reverse | |
| Ribosomal protein | 5′-AGCCGGTGTAAATGTTG |
| lateral stalk | AGCCT-3′ |
| subunit P1 forward | |
| Ribosomal protein | 5′-TCTTTGCTTCCACTTTC |
| lateral stalk | TTCTCCT-3′ |
| subunit P1 reverse | |

DNA targets

| | |
|---|---|
| Mitochondrial NADH | 5′-CACCCAAGAACAGGGTT |
| dehydrogenase | TGT-3′ |
| subunit 1 forward | |
| Mitochondrial NADH | 5′-TGGCCATGGGTATGTTG |
| dehydrogenase | TTAA-3′ |
| subunit 1 reverse | |
| 18SrRNA forward | 5′-TAGAGGGACAAGTGGCG |
| | TTC-3′ |
| 18SrRNA reverse | 5′-CGCTGAGCCAGTCAGTG |
| | T-3′ |

### Cellular ATP Content

The CelITox Green cytotoxicity assay (Promega, Madison, WI) was used to assess the degree of cytotoxicity and remained in all cases less than 15% under our experimental conditions. This demonstrates that the ATP content was not confounded by excessive cell death (FIG. 6 a). Briefly, cells were seeded in 96-well plates and stimulated as described above. In particular, 90 μL of suspended cells (2.5×10⁶ cells/mL) were seeded with 10 μL LIPS or 10 μL medium into each well. Then CelITox Green reagent was added and incubated for 15 min. Fluorescence was recorded at 520 nm. To assess the cellular ATP content the inventors performed a luciferase-based assay (Cell Titer Glo 2.0 Assay, Promega, Madison, WI) following the manufacturer's instructions. After assessment of cytotoxicity as described above, 100 μL CellTiter Glow 2.0 reagent was added to the wells and incubated for 10 min. Subsequently the luminescence was recorded.

Interaction of TFAM with Mitochondrial Transcription Factor 2B (TFB2M)

To quantify the mitochondrial protein interactions of TFAM with the Transcription Factor B2 (TFB2M) (Hillen (2017, Cell 171) a Proximity Ligation Assay (PLA) was performed. Cells were stimulated with LPS for 24 h or 48 h as describe above, centrifuged onto slides, fixed using 4% formaldehyde in PBS and permeabilized using 1% Triton X in PBS. After washing, epitopes were recovered by incubation in 1% SDS in PBS for 5 min. After subsequent washing unspecific binding sides were blocked. Primary antibodies against TFAM (1:50, sc-376672, Santa Cruz Biotechnology) and mitochondrial Transcription Factor 2B (1:50, 13676, Abcam,) were incubated for 1 h at room temperature. Proximity probes (anti-Mouse Plus; DU092001 and anti-Goat Minus; DU092006, both Sigma-Aldrich, each 1:5) were incubated for 1 h at room temperature, S3 splint and S3 backbone oligonucleotides (Biomers.net; Ulm, Germany) were hybridized, ligated and, after washing, amplified using rolling circle amplification combined with a compaction oligonucleotide (Table 2). S3 splint and S3 backbone oligonucleotides refer to oligonucleotide sequences described in Söderberg (2006), Nat Methods, 3. The rolling circle products were visualized with a detection oligonucleotide (Clausson (2015), Sci Rep 5, 12317). The compaction nucleotide is further described in Clausson (2015), Sci Rep 5, 12317). Nuclei were counterstained with DAPI and slides embedded in Antifade (S36938, Invitrogen). See Table 2 for the sequences of oligonucleotides used in the Proximity Ligation Assay.

Images were taken using an Olympus IX51 Widefield Microscope at a magnification of 40×. Maximum intensity projections were done on all z-stack images and processed with FIJI (National Institutes of Health, Bethesda, MD) for equal brightness and contrast. Images were submitted to a Cell Profiler pipeline quantifying the proximity ligation assay signals with single cell resolution.

TABLE 2
Sequences of oligonucleotides used
in the Proximity Ligation Assay

| Oligonucleotide | Sequence |
|---|---|
| S3 Backbone | 5′-phos-ctattag |
| | cgtccagtgaatgcg |
| | agtccgtctaagaga |
| | gtagtacagcagccg |
| | tcaagagtgtcta-3′ |
| S3 Splint | 5′-phos-gttctgt |
| | catatttaagcgtct |
| | taa-3′ |
| Compaction | 5′-agagagtagtac |
| Oligonucleotide | agcagccgtaaaaga |
| | gagtagtacagcagc |
| | cgtUUU-3′ |
| Detection | 5′-Atto550-cagt |
| Oligonucleotide | gaatgcgagtccgtc |
| | t-3′ |

### Statistical Analysis

This is the primary analysis of this data. A statistical power analysis was performed a priori for sample size estimation, based on results from previous studies (Vanasco (2014), Free Radic Biol Med 2014; 77; Yue (2015), J Cell Physiol 2015; 230). The effect size derived from these studies varied between 1.8 and 3.1. With an effect size of 1.8, an alpha error of 0.05, and a power of 0.80, the projected sample size needed (GPower 3.1, Germany) was estimated as n=10 for septic patients. Based on the longitudinal experimental design (measurement at 5 different time points), a number of 20 healthy volunteers was chosen. The characteristics of the subjects are reported as means with SD or medians with interquartile ranges (25^{th}; 75^{th} percentile) as appropriate. All continuous variables were tested for normal distribution using the Shapiro-Wilk-Test test and graphical assessment. Continuous independent variables were compared using the Student's t-test or the Mann-Whitney test. Continuous dependent variables were compared using the paired samples Student t-test or the Wilcoxon signed-rank test, as appropriate.

For graphical presentation of relative changes of TFAM expression at different time points in the LPS-stimulated PBMC experiments a procedure with 1000 resamples bootstrapping was performed to allow assigning measures of accuracy, i.e. corresponding 95% CIs.

A p-value of less than 0.05 was considered statistically significant. However, a meaningful trend may be also observed at slightly higher p-values e.g. at a p-value of around 0.06 or 0.11 in appreciation of the small sample size, but not at p-values above 0.15. All Cis were calculated with a coverage of 95%. All analyses were performed using SPSS (version 25, IBM, Chicago, IL, USA). For graphical presentations GraphPad Prism 8 (Graph-Pad, San Diego, CA, USA) was used.

For sufficient data reliability, all experimental results were independently evaluated by two scientists that did not conduct the underlying experiments.

### Results

Following LPS stimulation of PBMCs from 20 healthy volunteers (mean age: 32±9 years; 9 female, 11 male), supernatant TNF-α concentrations increased to a maximum of 1198±367 pg/m L) at 4 h (p<0.001; FIG. 1 a) followed by an increase in interleukin-6 to 1063±378 pg/mL (p<0.001, FIG. 1 b) and interleukin-10 to 674±222 pg/mL (p<0.001; FIG. 1 c) concentrations after 4 h and 24 h, respectively. Nuclear PGC-1α protein concentration increased following LPS stimulation (FIG. 1 d) and was accompanied by increased expression of TFAM mRNA (FIG. 1 e), suggesting increased mitochondrial biogenesis. LPS stimulation also evoked a 1.5-fold increase of cytonucleoplasmic immature TFAM protein at 24 h (p=0.003) and a 2-fold increase at 48 h (p<0.001) compared to unstimulated controls (FIG. 1 f and g; and FIGS. 3 a and b). These data suggest that the cells elicited an inflammatory response and activated mitochondrial biogenesis in response to a simulated infection by LPS.

Surprisingly, however, the amount of functionally important mature intramitochondrial TFAM diminished over time despite the increase in cytonucleoplasmic TFAM (FIG. 1 f and h; and FIGS. 3 c and d). Indeed, the amount of mature intramitochondrial TFAM had halved at 24 h (p=0.038) and further decreased to 40% at 48 h (p=0.002). Thus, LPS evoked an altered distribution of TFAM with a decreased amount of mature TFAM protein within mitochondria despite an increased amount of TFAM mRNA and immature TFAM protein in the cytonucleoplasm. In accord with the decreased amount of mature TFAM protein in mitochondria, the mitochondrial DNA copy number after LPS stimulation decreased by approximately 50% after 24 h (p=0.006) and 48 h (p<0.001) compared to unstimulated controls (FIG. 4 a). Mitochondrial mRNA expression of mitochondrial NADH dehydrogenase subunit 1, a mitochondrially-encoded subunit protein of complex I indispensable for mitochondrial function, decreased by approximately 30% after LPS stimulation at 24 h (p=0.047) and 40% at 48 h (p=0.008) compared to baseline (FIG. 4 b). The cellular ATP content nearly halved within 48 h after LPS stimulation compared to unstimulated controls (FIG. 4 c; p<0.001).

Stimulation of monocytes (PBMCs) with LPS reduced the number of TFAM-TFB2M interactions per cell significantly after 24 h and even more so after 48 h (reduction by 80%).

### Example 2. Septic Patients Show an Inflammatory Response and Increased Mitochondrial Biogenesis but have a Reduced Amount of Mature TFAM Protein in Mitochondria and a Mitochondrial Dysfunction

### Materials and Methods

The study was designed, and experiments were conducted as described in Example 1 above, unless specified differently.

The healthy control subjects were the same as described in Example 1 above. Septic patients were considered eligible if they fulfilled the criteria for sepsis as defined by the current Sepsis-3 definition and enrollment, written informed consent and blood sampling had been completed within the first 24 h after diagnosis of sepsis (Singer, (2016). JAMA 315). Exclusion criteria were age under 18 years, pregnancy, pre-existing anemia, known mitochondrial disorder, and the decision to withhold or withdraw life-sustaining treatment on the day of study inclusion. Ten septic patients admitted to the intensive care unit (ICU) of the University Hospital Knappschaftskrankenhaus Bochum between Dec. 3, 2018 and Feb. 28, 2019 were included. PBMCs of these patients were isolated as described in Example 1. However, cells of septic patients were not stimulated with LPS but directly processed and compared to cells of the healthy subjects before LPS stimulation (0 h). Clinical and demographic data were collected during routine care and extracted from hospital and ICU electronic health records. The inventors followed all patients for 30-day survival commencing from the day of the diagnosis of sepsis.

Baseline characteristics of the septic patients are shown in Table 3. The SOFA score at inclusion was 10±4 and 9 patients required norepinephrine for blood pressure support. Thirty-day mortality was 40%.

TABLE 3
Baseline characteristics of sepsis patients

| | Sepsis patients |
|---|---|
| Variable | (n = 10) |
| Age [yrs.], mean (±SD) | 58 ± 13 |
| Male sex | 6 (60%) |
| Body mass index [kg/m²] | 27.1 ± 3.8 |
| Site of infection | |
| Pneumonia | 5 (50%) |
| Abdominal infection | 3 (30%) |
| Skin and soft tissue infection | 1 (10%) |
| Urinary tract infection | 1 (10%) |
| Culture results | |
| Gram positive isolates only | 3 (30%) |
| Gram negative isolates only | 4 (40%) |
| Mixed bacterial isolates | 1 (10%) |
| Negative cultures | 2 (20%) |
| C-reactive protein concentration [mg/dL] | 22.9 ± 11.5 |
| Procalcitonin concentration [ng/mL] | 12.1 ± 24.2 |
| Leukocyte concentration [10⁹/L] | 17.5 ± 7.9 |
| Neutrophils [%] | 82.9 ± 5.3 |
| Eosinophils [%] | 1.6 ± 2.6 |
| Basophils [%] | 0.3 ± 0.2 |
| Lymphocytes [%] | 8.3 ± 4.2 |
| Monocytes [%] | 6.9 ± 4.0 |
| Simplified Acute Physiology Score | 43 ± 18 |
| Sepsis-related Organ Failure | 10 ± 4 |
| Assessment score | |
| Continuous hemofiltration/dialysis | 6 (60%) |
| Mechanical ventilation | 5 (50%) |
| Serum lactate concentration [mg/dL] | 1.8 ± 1.0 |
| Norepinephrine therapy | 9 (90%) |
| Death within 30 days | 4 (40%) |

Data are presented as n (%) or mean (±SD), as appropriate. The presented characteristics refer to baseline measurements recorded on study inclusion. There were no missing data.

To explore potential age-related bias, the inventors additionally performed a correlation analysis referring to age and several TFAM related measurements (Table 4).

TABLE 4
Exploration of an age-dependent effect on cytonucleoplasmatic
and mitochondrial TFAM concentrations. To assess a potential
age dependent effect on the cytonucleopasmic and mitochondrial
TFAM concentration, Spearman rank correlation was used
to describe a potential association.

| | | |
|---|---|---|
| Spearman correlation | r (95%-CI) | p-value |

Healthy controls

| | | | |
|---|---|---|---|
| Age vs. cyt. TFAM 0 h | −0.15 | (−0.56 to 0.32) | 0.53 |
| (control) | | | |
| Age vs. cyt. TFAM 24 h | 0.29 | (−0.19 to 0.66) | 0.22 |
| Age vs. cyt. TFAM 48 h | 0.11 | (0.36 to 0.54) | 0.65 |
| Age vs. mito TFAM 0 h | 0.06 | (−0.40 to 0.50) | 0.79 |
| (control) | | | |
| Age vs. mito TFAM 24 h | 0.03 | (−0.43 to 0.48) | 0.90 |
| Age vs. mito TFAM 48 h | 0.05 | (−0.41 to 0.49) | 0.84 |

Septic patients

| | | | |
|---|---|---|---|
| Age vs. cyt. TFAM | −0.01 | (−0.69 to 0.65) | 0.99 |
| Age vs. mito TFAM | −0.07 | (−0.61 to 0.59) | 0.86 |

The concentrations of cytokines (TNF-α, IL-6 and IL-10) were measured in the blood serum of healthy control subjects and septic patients.

### Results

Serum of septic patients demonstrated manifold greater concentrations of TNF-α (60 pg/mL±80; p<0.001; FIG. 7 a); interleukin-6 (428 pg/mL±423; p<0.001; FIG. 7 b), and interleukin-10 (35 pg/mL±11; p<0.001; FIG. 7 c) compared to serum of healthy controls (0 pg/mL 1; 2 pg/mL 4; 0 pg/mL±1 respectively). This was accompanied by an approximately 3-fold increase in nuclear PGC-1α protein concentration (FIG. 7 d; p<0.001) and of TFAM mRNA expression (FIG. 7 e; p<0.001) in the PBMCs of the septic patients, indicating an activated or enhanced mitochondrial biogenesis. In this context, the inventors also found a 1.8-fold greater expression of extramitochondrial TFAM protein (immature TFAM protein in the cytonucleoplasm) in sepsis patients compared to healthy controls (p=0.001; FIG. 7 f), as determined by Western blot. Surprisingly, however, the abundance of mature and functionally important intramitochondrial TFAM was approximately 80% less than in controls (p<0.001; FIG. 7 g), as also determined by Western blot.

In parallel with the findings in LPS stimulated PBMCs from healthy volunteer controls (FIG. 4 a-c), mitochondrial DNA copy number in septic patients' PBMCs was almost 70% lower (FIG. 8 a; p<0.001) than in healthy controls. Mitochondrial NADH dehydrogenase subunit 1 mRNA was over 80% lower (FIG. 8 b; p<0.001) and cellular ATP content was 60% lower (FIG. 8 c; p<0.001) in septic patients than in healthy controls. This demonstrates that the septic patients have a mitochondrial dysfunction.

To assess the functional relevance of intramitochondrial TFAM in PBMCs from sepsis patients, the inventors quantified the interactions of TFAM and mitochondrial Transcription Factor 2B (TF2BM), also known as the mitochondrial core transcription initiation complex (FIG. 9). The number of interactions of TFAM and TF2BM reflects the amount of active TFAM protein in the cells, i.e. in the mitochondria, because only the active and/or mature TFAM protein but not the inactive and/or immature TFAM protein is found within the mitochondrial core transcription initiation complex (Hillen (2017, Cell 171). Surprisingly, the inventors found a marked decrease of 74% in PLA signals per cell, as a measure of protein interactions (FIG. 9 a, p<0.001), when comparing PBMCs from septic patients (1.2 signals per cell; 95%-CI: 0.7 to 1.6, FIG. 9 c) to healthy controls (4.5 signals per cell; 95%-CI: 3.7 to 5.2, FIG. 9 b), suggesting a relevantly hampered mitochondrial transcription machinery in septic patients. These results are in line with the diminished mature mitochondrial TFAM protein levels determined by Western blot.

### Example 3. The Amount of Active and/or Mature TFAM Protein Indicates the Severity and Outcome of Sepsis-Related Organ Dysfunction

### Materials and Methods

The relationship between mature and/or active mitochondrial TFAM (protein amount or protein interactions) and the SOFA-score was evaluated using Spearman's correlation.

### Results

The inventors explored potential associations between molecular and clinical variables. The amount of mature mitochondrial TFAM protein and the SOFA score inversely correlated (r=−0.65; p=0.043), suggesting that the amount of mature mitochondrial TFAM protein may be used as an indicator of the severity of sepsis-related organ dysfunction and as survival predictor. As shown in FIG. 10a, the threshold for a particularly severe organ dysfunction (defined as SOFA>10) referred to a relative amount of mature TFAM (normalized to TRAP) below about 0.02.

The threshold for 30-day mortality referred to a relative amount of mature TFAM (normalized to TRAP) below about 0.02 as well.

Furthermore, the inventors analyzed the receiver operating characteristics using the ratio of the TRAP-normalized amount of mature mitochondrial TFAM protein (TFAMₘₜ) over the β-actin normalized amount of cytonucleoplasmic TFAM protein (TFAM_{cyto}) in respect to 30-day mortality (FIG. 11).

In contrast, immature TFAM protein (e.g. cytonucleoplasmic TFAM protein normalized to β-actin) showed no association with either clinical outcome.

Of special clinical interest, the diminished protein interactions of TFAM with TFB2M in PBMCs of septic patients inversely correlated with the SOFA score (r=−0.76; p=0.011; FIG. 12). This indicates an association between the amount of intramitochondrial mature and/or active TFAM protein and the severity of the sepsis-related organ dysfunction. Further strikingly, septic patients that survived for at least 30 days showed significantly more TFAM-TFB2M interactions than septic patients which have died within 30 days (FIG. 12). Moreover, a threshold of TFAM-TFB2M interactions (about 0.9 interactions/cell) could be determined above which all (5/5) septic patients survived and below which 80% (⅘) of the septic patients died.

### Example 4. Exemplary Uses of Active and/or Mature TFAM Levels for the Diagnostics of Septic Patients

### In-Situ Assay

Patients diagnosed with sepsis are selected, blood is drawn from these patients, and PBMCs are isolated from the blood, e.g., as described in Examples 1 and 2. The protein interaction of TFAM with TFB2M—which indicates the amount of active TFAM protein—is quantified by a proximity ligation assay (PLA), e.g. as described in Examples 1 and 2, or by proximity-dependent initiation of hybridization chain reaction (proxHCR). Alternatively, or in addition, the amount of mature TFAM protein may be determined in isolated mitochondria, e.g. as described in Examples 1 and 2. The amount of active and/or mature TFAM protein is then used as an indicator of the severity of the organ dysfunction (e.g. the SOFA score) and/or the outcome of the organ dysfunction (e.g. prognosing the survival of the patients), e.g., as described in Example 3.

### Point-of-Care (POC) Device

Patients diagnosed with sepsis are selected, and blood, e.g. a small amount such as 200 μl, is drawn from these patients, e.g., as described in Examples 1 and 2, and put on the POC. A filter depletes the whole blood from erythrocytes and the depleted blood flows into the microfluidic device. Cells there are lysed and the lysate flows over immobilized antibodies capturing e.g. TFAM. Then a second antibody comes in (after washing) and binds to TFB2M. Signal is amplified by means of rolling circle amplification (i.e. PLA) or hybridization chain reaction (proxHCR) and visualized by detection oligos (PLA) conjugated to horseradish peroxidase. In case of proxHCR the HRP is conjugated to the amplification oligonucleotides (see US20170009278). HRP can be used to generate a color signal (i.e. brown) that can be observed without a microscope through a “window”.

The amount of active TFAM protein (TFAM-TFB2M interactions) is then used as an indicator of the severity of the organ dysfunction (e.g. the SOFA score) and/or the outcome of the organ dysfunction (e.g. prognosing the survival of the patients), e.g., as described in Example 3.

### Evaluation of Treatment Success

The amount of mature mitochondrial TFAM protein and/or, preferably, the number of TFAM-TFB2M protein interactions in a septic patient, is determined by an in situ assay or a point-of-care device as described above. The septic patient is then treated, e.g. by intravenous administration of an antioxidant, and the treatment success is evaluated by drawing again blood from the patient and repeating the measurements. FIGS. 15 and 16 further demonstrate that the number of TFAM-TFB2M protein interactions, i.e. when measured repeatedly over time, is an excellent indicator of sepsis progression (or regression), and thus may be advantageously used for evaluating treatment success, and, if necessary, modifying the treatment regime.

### Experimental Treatment of a Specific Subgroup of Septic Patients

The amount of mature mitochondrial TFAM protein and/or, preferably, the number of TFAM-TFB2M protein interactions in a septic patient, is determined by an in situ assay or a point-of-care device as described above. When the level of mature and/or active TFAM protein is below a certain threshold and, accordingly, the prognosis of the sepsis is negative or unfavorable and/or the sepsis is very severe, and/or when the level of mature and/or active TFAM protein has decreased over time, the septic patient is assigned to Group A. Group A septic patients likely benefit from an experimental treatment such as intravenous administration of an antioxidant (e.g. Vitamin C). In contrast, group B septic patients, which refer to patients with a level of mature and/or active TFAM protein at least as high as the threshold, and/or in which the mature and/or active TFAM protein level has increased over time, may not benefit from such an experimental treatment which may have severe side effects, because the prognosis of the sepsis in these patients is already positive or favorable and/or the sepsis is not very severe. Thus, stratification of septic patients by the level of mature and/or active TFAM proteins may improve the overall survival of septic patients.

### Example 5. Discussion: The Role of Mature and/or Active TFAM in Sepsis and Further Diseases

The present study, as described, e.g., in Examples 1 to 4, reveals that the amount of mature TFAM protein in mitochondria is lower during the early inflammatory phase of sepsis, despite cellular upregulation of TFAM expression. Deprivation of intramitochondrial mature TFAM protein in turn was associated with reductions in mitochondrial DNA copy number, mitochondrial NADH dehydrogenase subunit 1 expression, and decreased cellular ATP content, all suggesting decreased cellular energy supply.

In line with other studies, the inventors found higher levels of TFAM mRNA and extramitochondrial immature TFAM protein in septic patients that was mirrored by LPS stimulation of PBMC from healthy volunteers within 24 to 48 hours (Vanasco (2014), Free Radic Biol Med 77; Haden (2007), Am J Respir Crit Care Med 176; Cherry (2014), J Biol Chem 289). One earlier study (Kraft (2019), Crit Care Med 47) has reported that TFAM mRNA was decreased at day 1 in septic patients but has increased again by days 3 and 5. The observations in the prior art and the findings of the inventors described herein indicate that TFAM mRNA (in contrast to mature and/or active TFAM protein) is not a robust biomarker which provides reproducible results across different patient cohorts and/or at different time-points of sepsis.

Tests of peripheral blood PBMCs have been proposed to offer valid information about “general” mitochondrial health (Kramer (2014), Redox Biol 2).

As shown by the progressive decrease both in mitochondrial DNA copy number and ATP content, mitochondrial function still deteriorated despite increased cellular TFAM concentrations. Remarkably, following LPS stimulation, impaired recovery of the mitochondrial respiratory chain was observed despite an increase in the nuclear transcription factor PGC-1α, a master regulator of mitochondrial biogenesis (Suliman (2004), Cardiovasc Res 64, 279-288). In this context, the results shed light on the intracellular maldistribution of TFAM in sepsis and endotoxemia resulting in a decreased level of mature intramitochondrial TFAM, although its source, i.e., extramitochondrial immature TFAM, is conserved or even increased.

It is possible that the observed findings are similar in cells of solid human organs, such as brain, kidney, heart, and liver.

A strength of the present study is, that the inventors could independently support the finding of a decreased level of mature intramitochondrial TFAM by a dramatically diminished TFAM-TFB2M protein interaction rate. This protein interaction occurs in the human mitochondrial transcription initiation complex. A decrease of this interaction was accompanied by a profoundly affected mitochondrial transcription and replication machinery, as shown by decreases in mitochondrial DNA copy number, mitochondrial NADH dehydrogenase subunit 1 mRNA expression, and cellular ATP content. Therefore, TFAM seems not to appear at its proper site of action where it is needed in septic patients.

As also summarized in FIG. 14, the above findings suggest that a diminished intramitochondrial concentration of mature TFAM protein impairs mitochondrial recovery and energetics. The data further demonstrate that a low amount of mature and/or active TFAM in mitochondria is associated with a very severe organ dysfunction and death from sepsis. In this context, a hampered mitochondrial protein import represents a possible mechanism evoking diminution of intramitochondrial TFAM. Furthermore, a higher proteolytic activity, e.g. by the LON protease, in the mitochondrion in sepsis and endotoxemia could also contribute to the above results (Harbauer (2014), Cell Metab 19; Matsushima (2010), Proc Natl Acad Sci USA 107).

Furthermore, the data suggest that the extent, and possibly the duration, of the apparent intracellular TFAM maldistribution represent a prognostic biomarker, since TFAM/TFB2M protein interactions strongly correlated with the SOFA score of the sepsis patients. Further studies in larger groups of patients may corroborate these findings. Furthermore, the above results may be of relevance for a broader variety of diseases given the impact of inflammation and mitochondrial dysfunction on a range of pathologies. Hence, it is contemplated that the level of mature and/or active TFAM protein in mitochondria may be used as diagnostic marker for indicating the presence and/or severity of a range of organ dysfunctions or prognosing the outcome of a range of organ dysfunctions, e.g. organ dysfunctions which are associated and/or caused by a mitochondrial dysfunction such, inter alia, as sepsis. Furthermore, controversial results, such as the inconsistent benefit of antioxidants in sepsis, may be reconciled by considering decreased intramitochondrial import of TFAM.

### Example 6. The Number of TFAM-TFB2M Interactions is a Better Indicator of the Severity of Sepsis than the TFAM mRNA Level

Septic patients were selected who all suffered from sepsis at a comparable degree of severity (as determined by the SOFA score) at day 1 of onset of sepsis (i.e. sepsis diagnosis). These patients did not represent a random sample of septic patients (in contrast to the patients in Example 3 and FIG. 12). This allowed the inventors to even more carefully address the question whether the number of TFAM-TFB2M interaction allows to monitor the severity of sepsis and the corresponding outcome. Furthermore, the informative values of the number of TFAM-TFB2M interaction (measured by PLA as described in Example 1) and TFAM mRNA levels (measured by qPCR as described in Example 1) were compared.

It was found that the number of TFAM-TFB2M interactions at day 1 (the day of the diagnosis of sepsis) did not predict whether the septic patients will be ICU-free after 1 week or whether they will die within 30 days (FIG. 15, top panel). As described in Example 3, it was found with a heterogeneous cohort of septic patients, that the number of TFAM-TFB2M interactions measured at day 1 of sepsis diagnosis was inversely correlated with the SOFA-score (a commonly used indicator of the severity of sepsis), and predicted the survival of the patients (FIG. 12). Together, these data corroborate the notion that the number of TFAM-TFB2M interactions is an excellent indicator of the degree of severity of sepsis, and that the outcome of the sepsis, e.g. survival and/or ICU-freedom of a septic patient is strongly associated with the degree of severity of the sepsis, i.e. observed at an earlier time-point.

The number of TFAM-TFB2M interactions was measured again after three days (at day 4) in the same patients (who all had a similar degree of severity of sepsis at day 1). During these three days, the course of the sepsis could change, i.e. the degree of severity of the sepsis may have increased or decreased for a given patient. In contrast to day 1 (FIG. 15, top panel), the number of TFAM-TFB2M interactions measured at day 4 (FIG. 15, bottom panel) was now predictive of the outcome of sepsis. In particular, patients who recovered fast and could be discharged from the intensive care unit (ICU) within one week (of diagnosis of sepsis) showed significantly more TFAM-TFB2M interactions than patients who were still in the ICU after one week or who died within 30 days. These data further corroborate the notion that the number of TFAM-TFB2M interactions is an excellent predictor of the outcome of sepsis which is associated with the degree of severity of the sepsis.

Furthermore, the predictive value of the difference (Δ) of the number of TFAM-TFB2M interactions per patient between day 4 and day 1 was analyzed (FIG. 16). The relative change of the number of interactions between day 4 and day 1 revealed strong and significant differences between the fast recovery group (ICU-free at one week) and the other two groups. In particular, an increase of the number of TFAM-TFB2M interactions predicted that a patient will be ICU-free within one week (of diagnosis of sepsis). In contrast, no change or a decrease of the number of TFAM-TFB2M interactions rather predicted that a patient will not be ICU-free within one week or even die within 30 days (of diagnosis of sepsis).

Furthermore, the difference (Δ) between day 4 and day 1 was negatively correlated with the SOFA score at each of days 1 to 7, wherein the strongest correlation was observed with the SOFA score at day 5 (p=0.014, r=−0.431).

These data further demonstrate that the number of TFAM-TFB2M interactions is very useful for monitoring the recovery from sepsis (regression of the sepsis) or the persistence or aggravation (progression) of the sepsis, and predicting the outcome of the sepsis. This allows to adjust the medical treatment (e.g. to add or withdraw a supportive drug based on a change in the level of mature and/or active TFAM protein, as described herein) and therefore improve the outcome of the sepsis and/or the overall survival of the patients.

TFAM mRNA levels at day 1 had no predictive power (FIG. 17, top panel). Furthermore, TFAM mRNA levels at day 4 (in contrast to TFAM-TFB2M interactions) were not predictive of ICU-freedom at one week (FIG. 17, bottom panel). At day 4, non-survivors had slightly higher TFAM mRNA levels than the fast recovery group (ICU-free at 1 week). Yet, the p-value was much higher (p=0.0173) than for the corresponding comparison based on TFAM-TFB2M interactions (p<0.001; where non-survivors had much less TFAM-TFB2M interactions than the fast recovery group at day 4; compare FIG. 17, bottom panel, and FIG. 15, bottom panel). Overall, the TFAM mRNA data were much more scattered (higher variation) than the TFAM-TFB2M interaction data. Altogether, these data indicate that the level of active and/or mature TFAM protein is a better indicator of the degree of severity of sepsis and the outcome of sepsis than the TFAM mRNA level.

In conclusion, these additional data further demonstrate that the level of mature and/or active TFAM protein (i.e. the number of the TFAM-TFB2M interactions) is a highly useful biomarker which correlates with the disease severity (e.g. the SOFA score) supporting risk stratification. Furthermore, said biomarker may be useful to discriminate people (e.g. septic patients) with a higher risk of death versus a lower risk of death. Within the framework of these statements, using TFAM-TFB2M interactions as a biomarker (e.g. by employing a PLA-assay) provides more reproducible and robust results compared to TFAM mRNA. Alternative biomarkers (including TFAM mRNA) do not provide such a great informative value. In particular, regarding TFAM mRNA, the inventors found, in addition to insufficient reproducibility, a too high a scatter and lack of functional relevance, so that it does not represent an appropriate alternative to the active and/or mature TFAM protein, e.g. as determined by TFAM-TFB2M interactions.

Serial TFAM-TFB2M measurements can indicate whether a patient is responding to the initial treatments or if a regimen change or termination needs to be considered. For example, the serial measurement of TFAM-TFB2M interactions may discriminate people (e.g. septic patients) that benefit from antioxidant treatment, by observing an increase in interactions during the therapy. Thus, it is may be preferable that TFAM-TFB2M interactions are measured every 24 to 48 hours to determine and describe an organ dysfunction trajectory and prognosis. Especially the evaluation of special adjunctive treatments focusing on sepsis seems prudent, since a lack of increase of TFAM-TFB2M interactions indicates that a treatment is ineffective which makes it reasonable that the treatment is safely stopped. In the other direction, patients with decreasing TFAM-TFB2M interactions may be candidates for extended adjuvant therapies (e.g. with a supporting drug as described herein).

1. (canceled)

2. The method of claim 53, further comprising
(c) determining a degree of severity of the organ dysfunction in the patient, wherein
(i) the degree of severity is low when the level of mature and/or active TFAM protein is abnormal but less than 40% lower in the sample from said patient than the amount determined for said reference sample, and/or
(ii) the degree of severity is high when the level of mature and/or active TFAM protein is at least about 40% or at least about 80% lower in the sample from said patient than the amount determined for said reference sample.

3-5. (canceled)

6. The method of claim 53, further comprising
(c) prognosing the outcome of said organ dysfunction in the patient, wherein
(i) a positive outcome is prognosed when the level of mature and/or active TFAM protein is abnormal but less than 40% lower in the sample from said patient than the amount determined for said reference sample,
and/or
(ii) a negative outcome is prognosed when the level of mature and/or active TFAM protein is at least about 40% or at least about 80% lower in the sample from said patient than the amount determined for said reference sample.

7. The method of claim 6, wherein the positive outcome comprises intensive care unit (ICU) freedom within one week and/or survival for at least one month, and/or the negative outcome comprises no ICU freedom for at least one week and/or death within one month.

8-11. (canceled)

12. An in vitro method for monitoring the course of an organ dysfunction in a patient, wherein said method comprises carrying out repeatedly the following step (a):
(a) determining the level of mature and/or active mitochondrial transcription factor A (TFAM) protein in a sample from said patient,
and carrying out at least once the following step (b):
(b) determining the course of the organ dysfunction in the patient, wherein
(i) when the level of mature and/or active TFAM protein has increased over time, recovery from the organ dysfunction is indicated,
(ii) when the level of mature and/or active TFAM protein remained similar over time, persistence of the organ dysfunction is indicated, and/or
(iii) when the level of mature and/or active TFAM protein has decreased over time, aggravation of the organ dysfunction is indicated.

13. (canceled)

14. A method of detecting an abnormal level of mature and/or active mitochondrial transcription factor A (TFAM) protein in a sample from a patient, wherein said method comprises
(a) measuring the level of mature and/or active TFAM protein in the sample; and
(b) determining whether the level in the sample is abnormal, wherein the level in the sample is determined to be abnormal if the level of mature and/or active TFAM protein is at least about 20%, at least about 50%, or at least about 70% lower than the amount determined for a reference sample.

15-17. (canceled)

18. The method of claim 14, wherein an abnormal level of mature and/or active TFAM protein in the sample is indicative of the presence of an organ dysfunction.

19. (canceled)

20. The method of claim 18, wherein a level of mature and/or active TFAM protein that is at least about 40% or at least about 80% lower in the sample from said patient than the amount determined for said reference sample is indicative of the presence of very severe organ dysfunction.

21-22. (canceled)

23. A method of treating an organ dysfunction in a patient, wherein said method comprises administering to the patient a supportive drug, wherein said supportive drug is selected from at least one drug from the group consisting of: (i) an antioxidant, (ii) human immunoglobulins, (iii) a chemotherapeutic agent, and (iii) a hydrocortisone, wherein
(a) said patient was reported as having an abnormal level of mature and/or active TFAM protein, and wherein said level of mature and/or active TFAM protein in a sample obtained from said patient was determined to be at least about 40% or at least about 80% lower than the amount determined for a reference sample, or
(b) said patient was reported as having a decreasing level of mature and/or active TFAM protein, and wherein said level of mature and/or active TFAM protein in samples repeatedly obtained from said patient has decreased by at least about 10% within one day.

24. (canceled)

25. The method of claim 18, wherein the organ dysfunction is sepsis.

26. The method of claim 18, wherein (a) the organ dysfunction is associated with and/or caused by an infection, and/or wherein the patient is suffering from an infection; and/or (b) wherein the organ dysfunction is associated with and/or caused by an inflammation, and/or wherein the patient is suffering from an inflammation.

27. (canceled)

28. The method of claim 18, wherein the organ dysfunction is associated with and/or caused by COVID19 and/or an infection with SARS-CoV-2.

29. (canceled)

30. The method of claim 14, wherein the level of mature and/or active TFAM protein corresponds to
(i) the amount of mature TFAM protein,
(ii) the number of interactions of TFAM protein with TFB2M protein, and/or
(iii) the ratio of the amount of mature TFAM protein over the amount of immature TFAM protein.

31. The method of claim 14, wherein the level of mature and/or active TFAM protein corresponds to the number of interactions of TFAM protein with TFB2M protein, and/or wherein measuring the level of mature and/or active TFAM protein comprises quantifying the interaction of TFAM protein with TFB2M protein.

32-33. (canceled)

34. The method of claim 31, wherein quantifying the interaction of TFAM protein with TFB2M protein comprises the steps of
(a) contacting said sample with a pair of binding molecules,
wherein one of said binding molecules specifically binds TFAM protein, and
wherein the other of said binding molecules specifically binds TFB2M protein, and
(b) generating a detectable signal when said two binding molecules are in close proximity to each other.

35. The method of claim 34, wherein one of the binding molecules is an antibody specifically binding TFAM protein and the other binding molecule is an antibody specifically binding TFB2M protein, wherein at least one of said binding molecules is an antibody conjugated to an oligonucleotide, and/or wherein at least one of said binding molecules is specifically bound by an antibody conjugated to an oligonucleotide; and/or wherein said step (b) of generating a detectable signal comprises the steps of
(i) generating an oligonucleotide template when said two binding molecules are in close proximity to each other, and
(ii) amplifying and/or extending said oligonucleotide template.

36-38. (canceled)

39. The method of claim 34, wherein said interaction is quantified by a proximity ligation assay (PLA) and/or a proximity-dependent initiation of hybridization chain reaction (proxHCR).

40. The method of claim 14, wherein the level of mature and/or active TFAM protein corresponds to the amount of mature TFAM protein in the mitochondria.

41. The method of claim 14, wherein said sample is a blood sample.

42-52. (canceled)

53. The method of claim 14, further comprising
(c) determining that an organ dysfunction is present in the patient when the level of mature and/or active TFAM protein is abnormal, and/or
determining that no organ dysfunction is present in the patient when the level of mature and/or active TFAM protein is not abnormal.

54. The method of claim 14, wherein the mature and/or active TFAM protein is a human mature TFAM protein with the sequence set forth in SEQ ID NO:4 or SEQ ID NO:8, or a protein that has at least 90% sequence identity to the sequence set forth in SEQ ID NO:4 or SEQ ID NO:8; and/or wherein the mature and/or active TFAM protein does not contain at the N-terminus the sequence set forth in SEQ ID NO:40 or a sequence that has at least 90% sequence identity to the sequence set forth in SEQ ID NO:40.
